(19) United States
(12) Patent Application Publication
(10) Pub. No.: US 2026/0294947 A1
(43) Pub. Date: Oct. 1, 2026

(54) FUCOSYLATED MAMMALIAN MILK OLIGOSACCHARIDE FOR USE IN THE PREVENTION OR TREATMENT OF SHIPPING FEVER

(71) Applicant: Inbiose N.V., Zwijnaarde (BE)

(72) Inventors: Joeri Beauprez, Zwijnaarde (BE); Wesley Carpentier, Zwijnaarde (BE); Ut Van Nguyen, Zwijnaarde (BE); Annelies Vercauteren, Zwijnaarde (BE); Jordi Lensink, Voorthuizen (NL); Marie Louise Miedema, Voorthuizen (NL); Mariska Van De Vosse, Voorthuizen (NL); Elian Elisabeth Marie-Louise Verscheijden, Voorthuizen (NL)

(21) Appl. No.: 19/480,753

(22) PCT Filed: May 3, 2024
(86) PCT No.: PCT/EP2024/062261

§ 371 Date: Oct. 31, 2025

(30) Foreign Application Priority Data
May 4, 2023 (EP) 23171613.5
May 4, 2023 (LU) LU504127
Jun. 14, 2023 (EP) 23179354.8

(51) Int. Cl. A61K 31/702; A61K 9/00; A61P 31/04

(52) U.S. Cl. CPC A61K 31/702; A61K 9/0095; A61P 31/04

(57) ABSTRACT

A fucosylated mammalian milk oligosaccharide (MMO) for use in a method for preventing and/or treating shipping fever in an animal, more specifically in a farmed animal or a domesticated animal. More specifically, the disclosure relates to a fucosylated mammalian milk oligosaccharide (MMO) for use in a method for preventing and/or treating shipping fever pneumonia and/or shipping fever diarrhea in an animal, more specifically in a farmed animal or a domesticated animal.

### CROSS-REFERENCE TO RELATED APPLICATIONS

This application is a national phase entry under 35 U.S.C. § 371 of International Patent Application PCT/EP2024/062261, filed May 3, 2024, designating the United States of America and published as International Patent Publication WO 2024/227921 A1 on Nov. 7, 2024, which claims the benefit under Article 8 of the Patent Cooperation Treaty of European Patent Convention patent application Ser. No. 23/171,613.5, filed May 4, 2023, Luxembourg Patent Application Serial No. LU504127, filed May 4, 2023, and European Patent Convention patent application Ser. No. 23/179,354.8, filed Jun. 14, 2023.

### TECHNICAL FIELD

This disclosure relates to a fucosylated mammalian milk oligosaccharide (MMO) for use in a method for preventing and/or treating shipping fever in an animal., more specifically in a farm animal or a domesticated animal. More specifically, the disclosure relates to a fucosylated mammalian milk oligosaccharide (MMO) for use in a method for preventing and/or treating shipping fever pneumonia in an animal., more specifically in a farm animal or a domesticated animal.

### BACKGROUND

The health status of a farm animal., i.e., an animal that is reared in an agricultural setting in order to produce various commodities such as food, hair or wool, has a tremendous impact on the economic endurance of farms due to the medical costs, animal losses and the long-term effects on the performance.

Farm animals are typically transported to rearing facilities. This transport not only brings animals close together (sometimes from different farms together), but it is well-known that such stressful transportation has negative effects on immunity, response to infection and inflammatory responses. Animals that are transported over long distances can develop shipping fever, which is a pulmonary disorder that has a multifactorial etiology of infectious agents (viral and/or bacterial; usually a primary viral infection is followed by a secondary bacterial superinfection), environment stress factors and host factors (Earley et al., 2017, Animal 11 (3): p. 486-492; Gaudino et al., 2022, Vet. Res. 53 (1); Maeda and Oikawa, 2019, Front. Vet. Sci. 6 (27)). Shipping fever may lead to pneumonia as well-documented for e.g., bovine and horse (Earley et al., 2017, Animal 11 (3): p. 486-492; Vivek Joshi et al., 2016, J. Immunol. And Immunopath. 18 (2)). Pneumonia is unfortunately the most common reason for death and poor performance in farm animals (Poulsen and Mcguirk, 2009, Vet. Clin. North. Am. Food Anim. Pract. 25 (1): p. 121-137; Arné et al., 2011, Int J. Microbiol., article 746356).

The development of an effective therapeutic for preventing and/or treating shipping fever, especially shipping fever pneumonia, is a medical and economical imperative.

### BRIEF SUMMARY

It was surprisingly found that a fucosylated MMO according to the disclosure proves to be an efficacious agent to prevent and/or treat shipping fever in an animal. The fucosylated MMO is not associated with adverse effects and is safe to use in animals.

In a first aspect, the disclosure provides a fucosylated MMO for use in a method for preventing and/or treating shipping fever in an animal, preferably wherein the animal is a farm animal or a domesticated animal.

In a second aspect, the disclosure provides a method for preventing and/or treating shipping fever in an animal, preferably a farm animal or a domesticated animal, wherein a fucosylated MMO according to the first aspect is administered to the animal.

In a third aspect, the disclosure provides the use of a fucosylated MMO according to the first aspect for the manufacture of a medicament for preventing and/or treating shipping fever in an animal, preferably a farm animal or a domesticated animal.

### BRIEF DESCRIPTION OF THE DRAWINGS

The following drawings will serve as further illustration and clarification of this disclosure and are not intended to be limiting in any way.

FIG. 1. Probability of first pneumonia incidence over time in control (placebo) and 2′-fucosyllactose (2′FL) treated animals of the first trial (veal calves).

FIG. 2. Probability of first pneumonia incidence over time in control (placebo) and 2′-fucosyllactose (2′FL) treated animals of the second trial (rearing calves).

### DETAILED DESCRIPTION

### Fucosylated MMO for Use in Preventing/Treating Shipping Fever

In a first aspect, the disclosure provides a fucosylated mammalian milk oligosaccharide (MMO) for use in a method for preventing and/or treating shipping fever in an animal., preferably wherein the animal is a farm animal or a domesticated animal., more preferably wherein the animal is a farm animal.

Preferably, the disclosure provides a composition comprising a fucosylated MMO, wherein the composition is for use in a method for preventing and/or treating shipping fever in an animal, preferably wherein the animal is a farm animal or a domesticated animal, more preferably wherein the animal is a farm animal. Throughout the disclosure and claims, the term “a fucosylated MMO” is preferably replaced with “a composition comprising a fucosylated MMO”, more preferably replaced with “a composition consisting essentially of a fucosylated MMO”, even more preferably replaced with “a composition consisting of a fucosylated MMO”.

### Shipping Fever

In an embodiment of the first aspect of the disclosure, the fucosylated MMO is for use in a method for preventing and/or treating shipping fever in an animal, preferably wherein the animal is a farm animal or a domesticated animal, more preferably wherein the animal is a farm animal.

In a preferred embodiment, the method is for preventing and/or treating shipping fever pneumonia. Shipping fever is a pulmonary disorder that is well-described and occurs in animals that are transported over long distances. Shipping fever has a multifactorial etiology of infectious agents (viral and/or bacterial; usually a primary viral infection is followed by a secondary bacterial superinfection), environment stress factors and host factors (Earley et al., 2017, Animal 11 (3): p. 486-492; Gaudino et al., 2022, Vet. Res. 53 (1); Maeda and Oikawa, 2019, Front. Vet. Sci. 6 (27)).

In this context of the disclosure, the animal is preferably a horse or a bovine, more preferably a bovine. When the animal is a bovine, than “shipping fever” is also referred to as “bovine respiratory disease (BRD)” or “bovine respiratory disease complex” (Vivek Joshi et al., 2016, J. Immunol. And Immunopath. 18 (2); Earley et al., 2017, Animal 11 (3): p. 486-492). Hence, when the animal is a bovine, the terms “shipping fever”, “bovine respiratory disease”, “BRD” and “bovine respiratory disease complex” can be interchangeably used in the context of this disclosure.

In a more preferred embodiment, the pneumonia is acute pneumonia. As understood by the skilled person, acute pneumonia entails inflammation of the alveoli and interstitial tissues of the lungs resulting in acute respiratory symptoms and signs including cough, breathlessness, chest pain and sputum production (Torres et al., 2021, Nature Reviews Disease Primers 7 (25); Ames, 2016, Vet. Clin. North Am. Food Anim. Pract. 13 (3): p. 379-391). The alveoli are filled with pus and fluid (in contrast to a healthy subject wherein the alveoli are filled with air when breathing) which makes breathing painful and limits oxygen intake. Chronic pneumonia is pneumonia that lasts at least 6 weeks; a radiograph of the chest usually shows diffuse or focal shadows.

In an additional and/or alternative preferred embodiment the method is for preventing and/or treating shipping fever diarrhea.

Surprisingly, it was found that a fucosylated mammalian milk oligosaccharide (MMO) according to the disclosure enhances the immune response in the treated subject, preferably enhances the T lymphocyte immunity in the treated subject, more preferably enhances the T lymphocyte activation in the treated subject.

The technical effect of treating a subject with a fucosylated MMO according to the disclosure hence encompasses an indirect mechanism of protection by stimulating the immune response in the treated subject as described herein to protect and/or fight off pathogens, rather than a direct mechanism wherein the fucosylated MMO would directly act on the pathogen and/or would block its entry by binding to an essential receptor for the pathogen. This is particularly beneficial in the context of shipping fever wherein animals are more prone to infection and generally have a weakened immunity due to the transport-induced stress. Furthermore, the indirect mechanism provides protection against a broad spectrum of pathogens rather than acting against a specific pathogen.

This technical effect can be assessed in a subject by the skilled person by using routinely available techniques in the art. A particularly useful and preferred method is flow cytometry (preferably fluorescence-activated cell sorting, i.e., FACS) to assess the activation of T cells of a subject. Herein, peripheral blood mononuclear cells can be isolated from a subject's blood sample using standard techniques such as density gradient centrifugation. To identify activated T lymphocytes, a panel of suitable activated T cell markers can be used during flow cytometry (preferably FACS) analysis. This includes the classical T cell markers such as CD3, CD4 and CD8, combined with one or more known activation markers such as CD69, CD25 and HLA-DR. An alternative method to assess the technical effect is illustrated in the Examples below (differential expression analysis of whole transcriptome).

### Subject

In an embodiment of the first aspect of the disclosure, the fucosylated MMO is for use in a method for preventing and/or treating shipping fever in an animal., preferably wherein the animal is a farm animal or a domesticated animal., more preferably a farm animal.

As understood by the skilled person, a “farm animal” refers to an animal that is reared in an agricultural setting in order to produce various commodities such as food (meat, organs, eggs, dairy products) and/or hair or wool. A farm animal is preferably selected from the group consisting of bovine, pig, sheep, goat, horse, donkey, mule, hinny, camelid (preferably llama or alpaca), rabbit and poultry: more preferably selected from the group consisting of bovine, pig, sheep, goat, horse, camelid (preferably llama or alpaca), rabbit and poultry; even more preferably selected from the group consisting of bovine, pig, sheep, goat, horse, camelid (preferably llama or alpaca) and poultry; even more preferably selected from the group consisting of bovine, pig, sheep, goat, camelid (preferably llama or alpaca) and poultry; even more preferably selected from the group consisting of bovine, pig, sheep, goat, and poultry; even more preferably selected from the group consisting of bovine, pig, sheep and goat; even more preferably selected from the group consisting of bovine, sheep and goat; most preferably the farm animal is a bovine.

Alternatively, a farm animal is preferably selected from the group consisting of bovine and pig, most preferably the farm animal is a bovine.

As understood by the skilled person, a “domesticated animal” refers to an animal that has been selectively bred and adapted over generations to live alongside humans. A domesticated animal in the context of the disclosure is preferably a companion animal., a horse, a donkey, a mule or a hinny; more preferably the domesticated animal is selected from the group consisting of a dog, a cat, a horse, a donkey, a mule and a hinny; even more preferably the domesticated animal is selected from the group consisting of a dog, a cat and a horse; most preferably the domesticated animal is a horse.

In the context of the disclosure, the term “poultry” refers to birds bred by humans for their eggs, meat and/or feathers. Throughout the disclosure and claims, the poultry preferably is a chicken, a quail, a turkey, a duck, a pigeon and a goose, more preferably a chicken, a quail or a turkey, more preferably chicken or a quail, most preferably a chicken. Throughout the disclosure and claims, the term “chicken” is preferably replaced with broiler.

In a preferred embodiment of the disclosure, the farm animal or domesticated animal is selected from the group consisting of bovine, pig, sheep, goat, horse, donkey, mule, hinny, camelid (preferably llama or alpaca), dog, cat, rabbit and poultry; more preferably selected from the group consisting of bovine, pig, sheep, goat, horse, donkey, mule, hinny, camelid (preferably llama or alpaca) and poultry; even more preferably selected from the group consisting of bovine, sheep, goat, horse, donkey, mule, hinny, camelid (preferably llama or alpaca) and poultry; even more preferably selected from the group consisting of bovine, horse, donkey, mule, hinny and poultry; even more preferably selected from the group consisting of bovine, horse, donkey, mule and hinny; even more preferably selected from the group consisting of bovine and horse; most preferably the animal is bovine.

In an additional and/or alternative preferred embodiment, the animal is a multigastric animal, preferably a ruminant or camelid, more preferably a ruminant.

Preferably, the camelid is a llama or an alpaca, more preferably the camelid is an alpaca.

Preferably, the ruminant is selected from the group consisting of bovine, sheep and goat, more preferably the ruminant is bovine.

In an alternative preferred embodiment, the animal is a monogastric animal., preferably selected from the group consisting of pig, horse, donkey, mule, hinny, rabbit and poultry, more preferably selected from the group consisting of pig, horse, donkey, mule, hinny and poultry, even more preferably selected from the group consisting of pig, horse, donkey, mule and hinny, even more preferably pig or horse, most preferably pig.

Throughout the description and claims, unless specifically stated otherwise, the terms “subject” and “animal” can be preferably replaced with “subject in the need thereof” and “animal in the need thereof”, respectively.

In a more preferred embodiment, the animal according to the disclosure is a non-adult animal. In an additional and/or alternative more preferred embodiment, the animal according to the disclosure is a young animal. Throughout the disclosure and claims, unless specifically stated otherwise, a “non-adult animal” can be preferably replaced with “juvenile animal” or “young animal”.

As understood by the skilled person:
a non-adult bovine is a calf;
a non-adult pig is a piglet;
a non-adult sheep is a lamb;
a non-adult goat is a kid;
a non-adult horse is a foal;
a non-adult llama is a cria;
a non-adult alpaca is a cria;
a non-adult dog is a puppy;
a non-adult cat is a kitten;
a non-adult rabbit is a kitten or a kit;
a non-adult poultry/chicken is a chick.

In an additional and/or alternative more preferred embodiment, the animal according to the disclosure is a preweaned animal.

In an additional and/or alternative more preferred embodiment, the animal according to the disclosure is an animal that is weaned.

As understood by the skilled person, weaning refers to the process of withdrawing the access of a young animal to milk and to gradually accustom the young animal to the adult diet.

Throughout the disclosure and claims, each embodiment disclosed in the context wherein the subject is a horse, is also disclosed for a subject being a donkey, a mule or a hinny.

Method for Preventing and/or Treating Shipping Fever

In an embodiment of the first aspect of the disclosure, the fucosylated MMO is for use in a method for preventing and/or treating shipping fever in an animal, preferably wherein the animal is a farm animal or a domesticated animal., more preferably a farm animal.

The animal is as described in the Section “Subject”.

In the context of the disclosure, the term “preventing” shipping fever as described herein, preferably means avoiding that the shipping fever occurs and/or decreasing the incidence of the shipping fever. In other words, “preventing” preferably refers to ameliorating the risk of suffering from the shipping fever. The term “prevention” and “prophylaxis” are interchangeably used in the context of the disclosure. The term “treating” shipping fever as described herein, preferably means inhibiting the shipping fever, e.g., arresting the development of the shipping fever; relieving the shipping fever, for example, causing regression of the shipping fever; and/or relieving a condition caused by or resulting from the shipping fever, e.g., relieving, preventing or treating symptoms of the shipping fever. In other words, “treating” preferably refers to decreasing the duration (number of days/weeks/months) the subject will suffer from the shipping fever), the risks, the complications and/or the severity of the shipping fever; this also encompasses the relief of the symptoms caused by the shipping fever.

In a preferred embodiment, the method according to the disclosure comprises administering an effective amount of the fucosylated MMO (it is referred to the Section “Oligosaccharide”) according to the disclosure to the animal (it is referred to the Section “Subject”). As understood by the skilled person, an “effective amount” is the amount of the oligosaccharide that is required to confer a therapeutic effect on the animal as described in the disclosure. Effective amounts vary, as recognized by those skilled in the art, depending on the subject, route of administration, excipient usage among other known factors.

In an additional and/or alternative preferred embodiment, the fucosylated MMO is administered to the animal at least once, preferably at least twice, between birth (0 weeks) and 5 weeks of age, preferably between birth and 4 weeks of age, more preferably between birth and 3 weeks of age.

It is particularly preferred in the context of the disclosure that the fucosylated MMO is administered to the animal at least once, preferably at least twice, more preferably daily:
in the three weeks, preferably two weeks, more preferably one week, preceding the day of transporting the animal; and/or
on the day of transporting the animal; and/or
in the three weeks, preferably two weeks, more preferably one week, after the day of transporting the animal.

More preferably the fucosylated MMO is administered to the animal at least once, preferably at least twice, more preferably daily:
in the three weeks, preferably two weeks, more preferably one week, preceding the day of transporting the animal; and/or
on the day of transporting the animal; or
on the day of transporting the animal; and/or
in the three weeks, preferably two weeks, more preferably one week, after the day of transporting the animal.

In a more preferred embodiment, the fucosylated MMO is administered at a daily dose of 0.0001-15.0 g, preferably 0.0001-10.0 g, more preferably 0.0001-5.0 g, even more preferably 0.0001-2.0 g, even more preferably 0.0001-1.0 g, even more preferably 0.0005-1.0 g, most preferably 0.0010-1.0 g. More preferably, the fucosylated MMO is administered at a daily dose of 0.001-15.0 g, preferably 0.001-10.0 g, more preferably 0.001-5.0 g, even more preferably 0.001-2.0 g, even more preferably 0.001-1.0 g, even more preferably 0.005-1.0 g, most preferably 0.010-1.0 g. Even more preferably, the fucosylated MMO is administered at a daily dose of 0.01-15.0 g, preferably 0.01-10.0 g, more preferably 0.01-5.0 g, even more preferably 0.01-2.0 g, even more preferably 0.01-1.0 g, even more preferably 0.015-1.0 g, most preferably 0.02-1.0 g. In the context of the disclosure, an amount of an oligosaccharide according to the disclosure expressed in a number of grams or milligrams per daily dose as used herein means that the amount of the oligosaccharide is such that when administering the daily dosage to a subject, the subject will be administered with the number of grams or milligrams of the oligosaccharide. In other words, if the daily dosage is, for example, 50 mg, then the subject receives in total 50 mg per day. This may be in one or more portions. So, if the daily dosage is 50 mg divided over 2 portions, then a single serving consists of 25 mg, a daily serving consists of 2 of such single servings.

In an additional and/or alternative more preferred embodiment, the fucosylated MMO is administered at a daily dose of 0.01-150.0 mg, preferably 0.01-125.0 mg, more preferably 0.01-100.0 mg, even more preferably 0.05-100.0 mg, even more preferably 0.1-100.0 mg, most preferably 0.1-80.0 mg, per kg bodyweight of the animal.

In an even more preferred embodiment, the fucosylated MMO is administered at a daily dose of 0.01-1.0 g, preferably 0.01-0.75 g, more preferably 0.01-0.65 g, even more preferably 0.01-0.60 g, even more preferably 0.01-0.50 g, even more preferably 0.015-0.50 g, even more preferably 0.015-0.40 g, even more preferably 0.015-0.30 g, even more preferably 0.015-0.20 g, most preferably 0.015-0.10 g. This embodiment is, for example, particularly preferred if the animal is a bovine or a horse, more preferably wherein the animal is a calf or a foal., most preferably wherein the animal is a calf.

In an additional and/or alternative even more preferred embodiment, the fucosylated MMO is administered at a daily dose of 0.1-50.0 mg, preferably 0.1-30.0 mg, more preferably 0.1-25.0 mg, even more preferably 0.1-20.0 mg, even more preferably 0.1-15.0 mg, even more preferably 0.1-10.0 mg, even more preferably 0.25-10.0 mg, most preferably 0.5-10.0 mg, per kg bodyweight of the animal. This embodiment is, for example, particularly preferred if the animal is a bovine or a horse, more preferably wherein the animal is a calf or a foal, most preferably wherein the animal is a calf.

In an alternative even more preferred embodiment, the fucosylated MMO is administered at a daily dose of 0.01-5.0 g, preferably 0.1-2.5 g, more preferably 0.1-1.5 g, even more preferably 0.1-1.2 g, even more preferably 0.2-1.2 g, even more preferably 0.3-1.2 g, even more preferably 0.4-1.2 g, most preferably 0.5-1.2 g. This embodiment is, for example, particularly preferred if the animal is a pig, a sheep, a goat or a camelid (preferably llama or alpaca), more preferably wherein the animal is a piglet, a lamb, a kid or a cria, even more preferably wherein the animal is a piglet, a lamb or a kid, most preferably wherein the animal is a piglet.

In an additional and/or alternative even more preferred embodiment, the fucosylated MMO is administered at a daily dose of 0.1-150.0 mg, preferably 0.1-125.0 mg, more preferably 1.0-125.0 mg, even more preferably 5.0-125.0 mg, even more preferably 5.0-100.0 mg, even more preferably 5.0-80.0 mg, most preferably 10.0-80.0 mg, per kg bodyweight of the animal. This embodiment is, for example, particularly preferred if the animal is a pig, a sheep, a goat or a camelid (preferably llama or alpaca), more preferably wherein the animal is a piglet, a lamb, a kid or a cria, even more preferably wherein the animal is a piglet, a lamb or a kid, most preferably wherein the animal is a piglet.

In an even more preferred embodiment, the fucosylated MMO is administered to the animal for at least 1 week, preferably at least 2 weeks, more preferably at least 3 weeks, even more preferably at least 4 weeks, most preferably at least 6 weeks.

In an additional and/or alternative even more preferred embodiment, the fucosylated MMO is administered to the animal for ≤32 weeks, preferably ≤28 weeks, more preferably ≤24 weeks, even more preferably ≤20 weeks, even more preferably ≤16 weeks, even more preferably ≤12 weeks, most preferably ≤6 weeks.

In an even more preferred embodiment, the fucosylated MMO is administered to the animal for 1-36, preferably 1-30, even more preferably 1-28, even more preferably 1-24, even more preferably 1-18, even more preferably 1-12, even more preferably 1-8, even more preferably 1-6, even more preferably 1-4, most preferably 2-4, consecutive weeks.

In an even more preferred embodiment, the fucosylated MMO is administered to the animal for:
if subject is bovine: 1-36, preferably 1-32, more preferably 1-30, even more preferably 1-28, even more preferably 1-24, even more preferably 1-18, even more preferably 1-12, even more preferably 2-12, most preferably 3-12, consecutive weeks;
if subject is a pig: 1-18, preferably 1-12, more preferably 1-8, even more preferably 1-6, even more preferably 1-4, most preferably 2-4, consecutive weeks;
if subject is a sheep: 1-36, preferably 1-32, more preferably 1-30, even more preferably 1-28, even more preferably 1-24, even more preferably 1-18, even more preferably 1-12, even more preferably 1-8, even more preferably 1-6, even more preferably 1-4, most preferably 2-4, consecutive weeks;
if subject is a goat: 1-36, preferably 1-32, more preferably 1-30, even more preferably 1-28, even more preferably 1-24, even more preferably 1-18, even more preferably 1-12, even more preferably 1-8, even more preferably 1-6, even more preferably 1-4, most preferably 2-4, consecutive weeks;
if subject is a horse: 1-18, preferably 1-12, more preferably 1-8, even more preferably 1-6, even more preferably 1-4, most preferably 2-4, consecutive weeks;
if subject is a camelid: 1-18, preferably 1-12, more preferably 1-8, even more preferably 1-6, even more preferably 1-4, most preferably 2-4, consecutive weeks;
if subject is a rabbit: 1-12, preferably 1-8, more preferably 1-6, even more preferably 1-4, most preferably 2-4, consecutive weeks;
if subject is a dog: 1-12, preferably 1-8, more preferably 1-6, even more preferably 1-4, most preferably 2-4, consecutive weeks;
if subject is a cat: 1-12, preferably 1-8, more preferably 1-6, even more preferably 1-4, most preferably 2-4, consecutive weeks;
if subject is poultry: 1-8, preferably 1-6, more preferably 1-4, most preferably 2-4, consecutive weeks.

Preferably, the fucosylated MMO according to the disclosure is administered at least once a week (i.e., at least one daily dosage is administered in a week), more preferably at least once every 3 days, even more preferably at least once every 2 days, most preferably at least once daily (i.e., a daily dosage is administered every day).

Surprisingly, it was found that a fucosylated MMO according to the disclosure is sufficient to obtain an effective therapeutic for preventing and/or treating shipping fever as described herein. The administration of other (oligo) saccharides is not required to obtain the therapeutic effect.

In this context of the disclosure, the disclosure provides a fucosylated MMO and a sialylated MMO, preferably a fucosylated MMO and two or more sialylated MMOs (i.e., a first sialylated MMO and a second sialylated MMO, optionally further sialylated MMO's are administered), more preferably a fucosylated MMO and two sialylated MMO's (i.e., a first sialylated MMO and a second sialylated MMO), for use in a method for preventing and/or treating shipping fever in an animal, preferably wherein the animal is a farm animal or a domesticated animal, more preferably wherein the animal is a farm animal. For the sake of clarity, the expression “more preferably two sialylated MMO's” in this context of the disclosure means that no additional sialylated MMO's are administered to the subject. In other words, the method for preventing and/or treating shipping fever in an animal as described herein comprises administering a fucosylated MMO as described herein and a sialylated MMO, preferably a fucosylated MMO and two or more sialylated MMOs (i.e., a first sialylated MMO and a second sialylated MMO, optionally further sialylated MMO's are administered), more preferably a fucosylated MMO and two sialylated MMO's (i.e., a first sialylated MMO and a second sialylated MMO).

It is preferred that the administered MMO's are administered as a mixture, more preferably as part of a composition, even more preferably as part of a composition according to Section “Composition”. The fucosylated MMO is as described herein (it is referred to the Section “Oligosaccharide). In a preferred embodiment, the sialylated MMO comprises a sialic acid, preferably a Neu5Ac, that is linked to a monosaccharide in an α-2,3-, α-2,6- or α-2,8-linkage, preferably an α-2,3- or an α-2,6-linkage, and wherein the monosaccharide is preferably selected from galactose, N-acetylglucosamine and Neu5Ac, more preferably the monosaccharide is galactose or N-acetylglucosamine, even more preferably the monosaccharide is galactose. The skilled person will understand that the expression “sialic acid is linked to a monosaccharide” refers to the situation wherein the sialic acid is bound to a monosaccharide through a glycosidic bond and wherein the sialic acid and monosaccharide are part of the oligosaccharide of the disclosure (which can comprise additional monosaccharide(s) than the sialic acid and the monosaccharide). In an additional and/or alternative preferred embodiment, the sialylated MMO consists of 3-8, more preferably 3-7, even more preferably 3-6, most preferably 3-5, monosaccharides. In an additional and/or alternative preferred embodiment, the sialylated MMO comprises a lactose, a lacto-N-biose (LNB) or N-acetyllactosamine (LacNAc) at its reducing end, preferably the sialylated MMO comprises lactose or LacNAc at is reducing end, most preferably the sialylated MMO comprises lactose at its reducing end.

A sialylated MMO comprising lactose at its reducing end is preferably selected from the group consisting of 3′-sialyllactose (3′SL), 6′-sialyllactose (6′SL), 3,6-disialyllactose, 6,6′-disialyllactose, 8,3-disialyllactose, 3′S-2′FL, 6′S-2′FL, 3′S-3-FL, 6′S-3-FL, LST a, LST b, LST c and LST d; more preferably selected from the group consisting of 3′SL, 6′SL, 3,6-disialyllactose, 6,6′-disialyllactose, 8,3-disialyllactose, LST a, LST b, LST c and LST d; even more preferably selected from the group consisting of 3′SL, 6′SL, LST a, LST b, LST c and LST d; most preferably wherein the sialylated MMO is 3′-sialyllactose (3′SL) or 6′-sialyllactose (6′SL). A sialylated MMO comprising lacto-N-biose (LNB) at its reducing end is preferably selected form the group consisting of 3′-sialyllacto-N-biose (3′SLNB), 6′-sialyllacto-N-biose (6′SLNB) and monofucosylmonosialyllacto-N-octaose (sialyl Lewis a); more preferably the sialylated MMO is 3′SLNB or 6′SLNB. A sialylated MMO comprising N-acetyllactosamine (LacNac) at its reducing end is preferably selected form the group consisting of 3′-sialyllactosamine (3′SLacNAc), 6′-sialyllactosamine (6′SLacNAc) and sialyl Lewis x; more preferably the sialylated MMO is 3′SLacNAc or 6′SLacNAc. In a more preferred embodiment, the sialylated MMO according to the disclosure is selected from a group consisting of 3′-sialyllactose (3′SL), 6′-sialyllactose (6′SL), 3,6-disialyllactose, 6,6′-disialyllactose, 8,3-disialyllactose, 3′S-2′FL, 6′S-2′FL, 3′S-3-FL, 6′S-3-FL, LST a, LST b, LST c, LST d, 3′-sialyllacto-N-biose (3′SLNB), 6′-sialyllacto-N-biose (6′SLNB), sialyl Lewis a, 3′-sialyllactosamine (3′SLacNAc), 6′-sialyllactosamine (6′SLacNAc) and sialyl Lewis x. More preferably the sialylated MMO is selected from the group consisting of 3′SL, 6′SL, LST a, LST b, LST c, LST d, 3′SLNB, 6′SLNB, sialyl Lewis a, 3′SLacNAc, 6′SLacNAc and sialyl Lewis x. Even more preferably the sialylated MMO is selected from the group consisting of 3′SL, 6′SL, 3′SLNB, 6′SLNB, 3′SLacNAc and 6′SLacNAc. Most preferably the sialylated MMO is 3′-sialyllactose (3′SL) or 6′-sialyllactose (6′SL). In an additional and/or alternative more preferred embodiment, the first sialylated MMO according to the disclosure is selected from a group consisting of 3′-sialyllactose (3′SL), 6′-sialyllactose (6′SL), 3,6-disialyllactose, 6,6′-disialyllactose, 8,3-disialyllactose, 3′S-2′FL, 6′S-2′FL, 3′S-3-FL, 6′S-3-FL, LST a, LST b, LST c, LST d, 3′-sialyllacto-N-biose (3′SLNB), 6′-sialyllacto-N-biose (6′SLNB), sialyl Lewis a, 3′-sialyllactosamine (3′SLacNAc), 6′-sialyllactosamine (6′SLacNAc) and sialyl Lewis x. Even more preferably the first sialylated MMO is selected from the group consisting of 3′SL, 6′SL, 3′S-2′FL, 6′S-2′FL, 3′S-3-FL, 6′S-3-FL, LST a, LST b, LST c, LST d, 3′SLNB, 6′SLNB, sialyl Lewis a, 3′SLacNAc, 6′SLacNAc and sialyl Lewis x. Even more preferably the first sialylated MMO is selected from the group consisting of 3′SL, 6′SL, LST a, LST b, LST c, LST d, 3′SLNB, 6′SLNB, sialyl Lewis a, 3′SLacNAc, 6′SLacNAc and sialyl Lewis x. Even more preferably the first sialylated MMO is selected from the group consisting of 3′SL, 6′SL, 3′SLNB, 6′SLNB, 3′SLacNAc and 6′SLacNAc. Even more preferably the first sialylated MMO is 3′-sialyllactose (3′SL) or 6′-sialyllactose (6′SL), most preferably the first sialylated MMO is 3′SL. In an additional and/or alternative more preferred embodiment, the second sialylated MMO according to the disclosure is selected from a group consisting of 3′-sialyllactose (3′SL), 6′-sialyllactose (6′SL), 3,6-disialyllactose, 6,6′-disialyllactose, 8,3-disialyllactose, 3′S-2′FL, 6′S-2′FL, 3′S-3-FL, 6′S-3-FL, LST a, LST b, LST c, LST d, 3′-sialyllacto-N-biose (3′SLNB), 6′-sialyllacto-N-biose (6′SLNB), monofucosylmonosialyllacto-N-octaose (sialyl Lewis a), 3′-sialyllactosamine (3′SLacNAc), 6′-sialyllactosamine (6′SLacNAc) and sialyl Lewis x. Even more preferably the second sialylated MMO is selected from the group consisting of 3′SL, 6′SL, LST a, LST b, LST c, LST d, 3′SLNB, 6′SLNB, sialyl Lewis a, 3′SLacNAc, 6′SLacNAc and sialyl Lewis x. Even more preferably the second sialylated MMO is selected from the group consisting of 3′SL, 6′SL, LST a, LST b, LST c, LST d, 3′SLNB, 6′SLNB, sialyl Lewis a, 3′SLacNAc, 6′SLacNAc and sialyl Lewis x. Even more preferably the second sialylated MMO is selected from the group consisting of 3′SL, 6′SL, 3′SLNB, 6′SLNB, 3′SLacNAc and 6′SLacNAc. Even more preferably the second sialylated MMO is 6′-sialyllactose (6′SL) or 3′-sialyllactose (3′SL), most preferably the second sialylated MMO is 6′SL.

When a fucosylated MMO and a first sialylated MMO are administered to the animal, the fucosylated MMO and the first sialylated MMO are preferably administered on the same day, more preferably administered simultaneously. Further, it is a preferred embodiment that the amount of the first sialylated MMO administered to the animal is 0.1-30.0, preferably 0.1-25.0, more preferably 0.1-20.0, even more preferably 0.1-15.0, even more preferably 0.2-15.0, even more preferably 0.3-15.0, even more preferably 0.4-15.0, most preferably 0.5-15.0, times the amount of the fucosylated MMO administered to the animal. Further, it is a more preferred embodiment that the amount of the fucosylated MMO administered to the animal is lower than the amount of the first sialylated MMO, preferably the amount of the fucosylated MMO is 1.1 to 10.0, preferably 1.5-7.5, more preferably 2.5-7.5, most preferably 4.0-7.0, times lower than the amount of the first sialylated MMO.

When a fucosylated MMO, a first sialylated MMO and a second sialylated MMO according to the disclosure are administered to the animal, the fucosylated MMO, the first sialylated MMO and the second sialylated MMO are preferably administered on the same day, more preferably administered simultaneously. Further, it is a preferred embodiment that the ratio between the amount of the first sialylated MMO administered to the animal and the amount of the second sialylated MMO administered to the animal is similar to this ratio as found in the mother's milk of the same species (preferably as described in the Section “Composition”). More preferably, the amount of the first sialylated MMO administered to the animal is higher than the amount of the second sialylated MMO, preferably the amount of the first sialylated MMO is 1.1 to 40.0, more preferably 1.1 to 35.0, most preferably 1.5 to 35.0, times higher than the amount of the second sialylated MMO. Even more preferably, the amount of the first sialylated MMO administered to the animal is higher than the amount of the second sialylated MMO, preferably the amount of the first sialylated MMO is 1.1 to 6.0, more preferably 1.1 to 5.0, even more preferably 1.25 to 4.0, even more preferably 1.25-3.0, even more preferably 1.5 to 3.0, most preferably 1.5-2.5, times higher than the amount of the second sialylated MMO. It is an additional and/or alternative preferred embodiment that the amount of the first sialylated MMO administered to the animal is 0.1-30.0, preferably 0.1-25.0, more preferably 0.1-20.0, even more preferably 0.1-15.0, even more preferably 0.2-15.0, even more preferably 0.3-15.0, even more preferably 0.4-15.0, most preferably 0.5-15.0, times the amount of the fucosylated MMO administered to the animal. Preferably, the amount of the fucosylated MMO administered to the animal is lower than the amount of the first sialylated MMO, preferably the amount of the fucosylated MMO is 1.1 to 10.0, preferably 1.5-7.5, more preferably 2.5-7.5, most preferably 4.0-7.0, times lower than the amount of the first sialylated MMO. It is an additional and/or alternative preferred embodiment that the amount of the second sialylated MMO administered to the animal is 0.1-30.0, preferably 0.1-25.0, more preferably 0.1-20.0, even more preferably 0.1-15.0, even more preferably 0.2-15.0, even more preferably 0.3-15.0, even more preferably 0.4-15.0, most preferably 0.5-15.0, times the amount of the fucosylated MMO administered to the animal. Preferably, the amount of the fucosylated MMO administered to the animal is lower than the amount of the second sialylated MMO, preferably the amount of the fucosylated MMO is 1.1 to 10.0, preferably 1.5-7.5, more preferably 1.5-5.0, most preferably 2.5-5.0, times lower than the amount of the second sialylated MMO.

It is a more preferred embodiment that the total amount of both first sialylated MMO and second sialylated MMO is 0.1-30.0, preferably 0.1-25.0, more preferably 0.1-20.0, even more preferably 0.1-15.0, even more preferably 0.2-15.0, even more preferably 0.3-15.0, even more preferably 0.4-15.0, most preferably 0.5-15.0, times the amount of the fucosylated MMO administered to the animal. Preferably, the amount of the fucosylated MMO administered to the animal is lower than the total amount of both first sialylated MMO and second sialylated MMO, preferably the amount of the fucosylated MMO is 1.1 to 15.0, preferably 1.5 to 15.0, more preferably 2.5 to 15.0, even more preferably 5.0 to 15.0, even more preferably 5.0 to 12.5, most preferably 5.0 to 10.0, times lower than the total amount of both first sialylated MMO and second sialylated MMO. It is an even more preferred embodiment that the amount of all sialylated MMOs that are administered to the animal is 0.1-30.0, preferably 0.1-25.0, more preferably 0.1-20.0, even more preferably 0.1-15.0, even more preferably 0.2-15.0, even more preferably 0.3-15.0, even more preferably 0.4-15.0, most preferably 0.5-15.0, times the amount of the fucosylated administered to the animal. Preferably, the amount of the fucosylated MMO administered to the animal is lower, preferably 1.1 to 15.0 times lower, more preferably 1.5 to 15.0 times lower, even more preferably 2.5 to 15.0 times lower, even more preferably 5.0 to 15.0 times lower, even more preferably 5.0 to 12.5 times lower, most preferably 5.0 to 10.0 times lower, than the amount of all sialylated MMOs administered to the animal.

In this context of the disclosure, the sialylated MMO/first sialylated MMO/second sialylated MMO is administered at a daily dose of 0.0001-15.0 g, preferably 0.0001-10.0 g, more preferably 0.0001-5.0 g, even more preferably 0.0001-2.0 g, even more preferably 0.0001-1.0 g, even more preferably 0.0005-1.0 g, most preferably 0.0010-1.0 g. More preferably, the sialylated MMO/first sialylated MMO/second sialylated MMO is administered at a daily dose of 0.001-15.0 g, preferably 0.001-10.0 g, more preferably 0.001-5.0 g, even more preferably 0.001-2.0 g, even more preferably 0.001-1.0 g, even more preferably 0.005-1.0 g, most preferably 0.010-1.0 g. Even more preferably, the sialylated MMO/first sialylated MMO/second sialylated MMO is administered at a daily dose of 0.01-15.0 g, preferably 0.01-10.0 g, more preferably 0.01-5.0 g, even more preferably 0.01-2.0 g, even more preferably 0.01-1.0 g, even more preferably 0.015-1.0 g, most preferably 0.02-1.0 g.

In an additional and/or alternative more preferred embodiment, the sialylated MMO/first sialylated MMO/second sialylated MMO is administered at a daily dose of 0.01-150.0 mg, preferably 0.01-125.0 mg, more preferably 0.01-100.0 mg, even more preferably 0.05-100.0 mg, even more preferably 0.1-100.0 mg, most preferably 0.1-80.0 mg, per kg bodyweight of the animal.

In an even more preferred embodiment, the sialylated MMO/first sialylated MMO is administered at a daily dose of 0.01-1.0 g, preferably 0.01-0.75 g, more preferably 0.025-0.75 g, even more preferably 0.05-0.75 g, most preferably 0.05-0.50 g. This embodiment is, for example, particularly preferred if the animal is a bovine or a horse, more preferably wherein the animal is a calf or a foal., most preferably wherein the animal is a calf. In an additional and/or alternative even more preferred embodiment, the sialylated MMO/first sialylated MMO is administered at a daily dose of 0.1-100.0 mg, preferably 0.1-75.0 mg, more preferably 0.5-75.0 mg, even more preferably 1.0-75.0 mg, even more preferably 2.5-75.0 mg, most preferably 2.5-50.0 mg, per kg bodyweight of the animal. This embodiment is, for example, particularly preferred if the animal is a bovine or a horse, more preferably wherein the animal is a calf or a foal, most preferably wherein the animal is a calf.

In an alternative even more preferred embodiment, the sialylated MMO/first sialylated MMO is administered at a daily dose of 0.01-5.0 g, preferably 0.1-2.5 g, more preferably 0.1-2.0 g, even more preferably 0.1-1.5 g, even more preferably 0.2-1.5 g, even more preferably 0.3-1.5 g, even more preferably 0.4-1.5 g, most preferably 0.5-1.5 g. This embodiment is, for example, particularly preferred if the animal is a pig, a sheep, a goat or a camelid (preferably llama or alpaca), more preferably wherein the animal is a piglet, a lamb, a kid or a cria, even more preferably wherein the animal is a piglet, a lamb or a kid, most preferably wherein the animal is a piglet. In an additional and/or alternative even more preferred embodiment, the sialylated MMO/first sialylated MM is administered at a daily dose of 0.1-150.0 mg, preferably 0.5-150.0 mg, more preferably 1.0-150.0 mg, even more preferably 1.0-125.0 mg, even more preferably 2.5-125.0 mg, even more preferably 5.0-125.0 mg, even more preferably 10.0-125.0 mg, even more preferably 25.0-125.0 mg, even more preferably 50.0-125.0 mg, most preferably 75.0-125.0 mg, per kg bodyweight of the animal. This embodiment is, for example, particularly preferred if the animal is a pig, a sheep, a goat or a camelid (preferably llama or alpaca), more preferably wherein the animal is a piglet, a lamb, a kid or a cria, even more preferably wherein the animal is a piglet, a lamb or a kid, most preferably wherein the animal is a piglet.

In an additional and/or alternative even more preferred embodiment, the second sialylated MMO is administered at a daily dose of 0.01-1.0 g, preferably 0.01-0.75 g, more preferably 0.025-0.75 g, even more preferably 0.025-0.50 g, most preferably 0.025-0.25 g. This embodiment is, for example, particularly preferred if the animal is a bovine or a horse, more preferably wherein the animal is a calf or a foal, most preferably wherein the animal is a calf. In an additional and/or alternative even more preferred embodiment, the second sialylated MMO is administered at a daily dose of 0.1-100.0 mg, preferably 0.1-75.0 mg, more preferably 0.25-75.0 mg, even more preferably 0.5-75.0 mg, even more preferably 1.0-75.0 mg, even more preferably 1.0-50.0 mg, most preferably 1.0-25.0 mg, per kg bodyweight of the animal. This embodiment is, for example, particularly preferred if the animal is a bovine or a horse, more preferably wherein the animal is a calf or a foal, most preferably wherein the animal is a calf.

In an alternative even more preferred embodiment, the second sialylated MMO is administered at a daily dose of 0.01-5.0 g, preferably 0.1-2.5 g, more preferably 0.1-2.0 g, even more preferably 0.1-1.5 g, even more preferably 0.1-1.2 g, even more preferably 0.15-1.2 g, even more preferably 0.2-1.2 g, most preferably 0.25-1.2 g. This embodiment is, for example, particularly preferred if the animal is a pig, a sheep or a goat, more preferably wherein the animal is a piglet, a lamb or a kid, most preferably wherein the animal is a piglet. In an additional and/or alternative even more preferred embodiment, the second sialylated MMO is administered at a daily dose of 0.1-150.0 mg, preferably 1.0-150.0 mg, more preferably 1.0-125.0 mg, even more preferably 2.5-125.0 mg, even more preferably 5.0-125.0 mg, even more preferably 10.0-125.0 mg, even more preferably 25.0-125.0 mg, even more preferably 25.0-100.0 mg, most preferably 25.0-80.0 mg, per kg bodyweight of the animal. This embodiment is, for example, particularly preferred if the animal is a pig, a sheep, a goat or a camelid (preferably llama or alpaca), more preferably wherein the animal is a piglet, a lamb, a kid or a cria, even more preferably wherein the animal is a piglet, a lamb or a kid, most preferably wherein the animal is a piglet.

While additional (oligo) saccharides can be administered according to the disclosure, the disclosure relates to the fact that only the fucosylated MMO according to the disclosure is required for achieving an efficient therapeutic effect as described herein.

It is hence particularly preferred that the method for preventing and/or treating shipping fever in an animal as described herein, does not comprise administering lacto-N-tetraose (LNT). In an additional and/or alternative preferred embodiment, the method for preventing and/or treating shipping fever in an animal as described herein, does not comprise administering lacto-N-neotetraose (LNnT). In an additional and/or alternative preferred embodiment, the method for preventing and/or treating shipping fever in an animal as described herein, does not comprise administering N-acetyllactosamine.

In an additional and/or alternative preferred embodiment, the method for preventing and/or treating shipping fever in an animal as described herein, does not comprise administering an oligosaccharide containing N-acetyllactosamine, preferably does not comprise a saccharide containing N-acetyllactosamine. In an additional and/or alternative preferred embodiment, the method for preventing and/or treating shipping fever in an animal as described herein, does not comprise administering 3′sialyllactose. In an additional and/or alternative preferred embodiment the method for preventing and/or treating shipping fever in an animal as described herein, does not comprise administering 6′sialyllactose. More preferably, the method for preventing and/or treating shipping fever in an animal as described herein, does not comprise administering a sialylated mammalian milk oligosaccharide, even more preferably does not comprise administering a sialylated oligosaccharide, most preferably does not comprise administering a sialylated saccharide. In an additional and/or alternative preferred embodiment, the method for preventing and/or treating shipping fever in an animal as described herein, does not comprise administering a fructo-oligosaccharide (FOS). In an additional and/or alternative preferred embodiment, the method for preventing and/or treating shipping fever in an animal as described herein, does not comprise administering a galactose-oligosaccharide (GOS). In an additional and/or alternative preferred embodiment, the method for preventing and/or treating shipping fever in an animal as described herein, does not comprise administering an isomaltooligosaccharide.

In an even more preferred embodiment, the method for preventing and/or treating shipping fever in an animal as described herein, does not comprise administering a neutral non-fucosylated MMO. In an even more preferred embodiment, the method for preventing and/or treating shipping fever in an animal as described herein, comprises administering a fucosylated MMO according to the disclosure and no additional MMOs (preferably no additional oligosaccharides, more preferably no additional saccharides). In an even more preferred embodiment, the method for preventing and/or treating shipping fever in an animal as described herein, only comprises administering the fucosylated MMO as described herein.

In another preferred embodiment, the method for preventing and/or treating shipping fever in an animal as described herein further comprises administering a microbial strain that does not use, preferably is not capable to use, the fucosylated MMO as carbon source. In an alternative preferred embodiment, the method for preventing and/or treating shipping fever in an animal as described herein does not comprise administering a microbial strain.

### Oligosaccharide

A fucosylated mammalian milk oligosaccharide (MMO) according to the disclosure is a MMO comprising a fucose.

As understood by the skilled person, mammalian milk oligosaccharides (MMOs) comprise oligosaccharides present in milk found in any phase during lactation including colostrum milk from humans (i.e., human milk oligosaccharides or HMOs) and mammals including but not limited to cows (Bos Taurus), sheep (Ovis aries), goats (Capra aegagrus hircus), bactrian camels (Camelus bactrianus), horses (Equus ferus caballus), pigs (Sus scropha), dogs (Canis lupus familiaris), ezo brown bears (Ursus arctos yesoensis), polar bear (Ursus maritimus), Japanese black bears (Ursus thibetanus japonicus), striped skunks (Mephitis mephitis), hooded seals (Cystophora cristata), Asian elephants (Elephas maximus), African elephant (Loxodonta africana), giant anteater (Myrmecophaga tridactyla), common bottlenose dolphins (Tursiops truncates), northern minke whales (Balaenoptera acutorostrata), tammar wallabies (Macropus eugenii), red kangaroos (Macropus rufus), common brushtail possum (Trichosurus vulpecula), koalas (Phascolarctos cinereus), eastern quolls (Dasyurus viverrinus), platypus (Ornithorhynchus anatinus) (Urashima T. et al., 2011, Milk Oligosaccharides, Nova Biomedical Books, New York ISBN 978-1-61122-831-1; Coppa et al., 2013, Ital. J. Pediatr. 2013, 39 (2)). A replete amount of milk saccharide structures have been elucidated so far. The majority of milk oligosaccharides found in animals such as mammals and humans comprise lactose at the reducing end (Urashima et al., 2011). Other milk oligosaccharides comprise N-acetyllactosamine (Gal-β1,4-GlcNAc) or lacto-N-biose (Gal-β1,3-GlcNAc) at the reducing end (Urashima et al., 2011; Wrigglesworth et al., 2020, PLOS ONE 15 (12); Urashima et al., 2013, Biosci. Biotechnol. Biochem 77 (3): p. 455-466; Wei et al., 2018, Sci. Rep. 8:4688). Examples hereof are 3-FLN (Gal-β1,4-(Fuc-α1,3-) GlcNAc; also known as Lewis x antigen), 3′-SLN (Neu5Ac-α2,3-Gal-β1,4-GlcNAc), 6′-SLN (Neu5Ac-α2,6-Gal-β1,4-GlcNAc) (Urashima et al., 2011; Wrigglesworth et al., 2020; Wei et al., 2018). Further, milk saccharides comprise milk glycosaminoglycans (GAGs; Coppa et al., 2013; Rai et al., 2021, Int. J. Biol. Macromolecules, 193 (A): p. 137-144). Antigens of the human ABO blood group system such as the A determinant (GalNAc-α1,3 (Fuc-α1,2)-Gal), B determinant (Gal-α1,3 (Fuc-α1,2)-Gal) and H-determinant (Fuc-α1,2-Gal) have been shown to be present in saccharides of the milk of mammals (e.g., A tetrasaccharide in a lion and leopard; Wrigglesworth et al., 2020).

Throughout the disclosure and claims, a “mammalian milk oligosaccharide (MMO)” is preferably a “human milk oligosaccharide (HMO)”.

In the context of the disclosure, the term “oligosaccharide” preferably refers to a saccharide containing 2 up to and including 20 monosaccharides, i.e., the degree of polymerization (DP) is 2-20, more preferably refers to a saccharide containing 3 up to and including 20 monosaccharides, i.e., the degree of polymerization (DP) is 3-20. In on other words, the term “oligosaccharide” preferably refers to a saccharide consisting of 2-20, more preferably 3-20, monosaccharide units, which are linked to each other via glycosidic bonds in a linear or in a branched structure. The linkage (i.e., glycosidic linkage), between two sugar units, i.e., two monosaccharides, can be expressed, for example, as 1,4, 1->4, or (1-4), which are used interchangeably herein.

More preferably, an oligosaccharide as described herein consists of 3-8, even more preferably 3-7, even more preferably 3-6, most preferably 3-5, monosaccharides. For the sake of clarity, the expression “x-y” refers to a range from and including x to and including y. For example, 3-5 monosaccharides means that 3, 4 or 5 monosaccharides are present.

Preferably, the fucosylated MMO according to the disclosure contains only one fucose, i.e., the MMO consists of two or more monosaccharides and only one of the monosaccharides is a fucose.

In a preferred embodiment of the disclosure, the fucosylated MMO is a neutral oligosaccharide. A “neutral” oligosaccharide as used herein and as generally understood in the state of the art is an oligosaccharide that has no negative charge originating from a carboxylic acid group.

In an additional and/or alternative preferred embodiment, the fucosylated MMO comprises a fucose that is linked to a monosaccharide in an α-1,2-, α-1,3- or α-1,4-linkage, preferably an α-1,2- or an α-1,3-linkage, more preferably an α-1,2-linkage, and wherein the monosaccharide is preferably selected from glucose, N-acetylglucosamine and galactose, more preferably the monosaccharide is glucose or galactose, even more preferably the monosaccharide is galactose. The skilled person will understand that the expression “fucose is linked to a monosaccharide” refers to the situation wherein the fucose is bound to a monosaccharide through a glycosidic bond and wherein the fucose and monosaccharide are part of the oligosaccharide of the disclosure (which can comprise additional monosaccharide(s) than the fucose and the monosaccharide).

In an additional and/or alternative preferred embodiment, the fucosylated mammalian milk oligosaccharide comprises a lactose, a lacto-N-biose (LNB) or N-acetyllactosamine (LacNAc) at its reducing end, preferably the fucosylated oligosaccharide comprises lactose or LacNAc at is reducing end, more preferably the fucosyalted oligosaccharide comprises lactose at its reducing end.

A fucosylated oligosaccharide comprising lactose at its reducing end is preferably selected form the group consisting of 2′-fucosyllactose (2′FL), 3-fucosyllactose (3-FL), difucosyllactose (diFL), lacto-N-fucopentaose I (LNFP I), blood group A antigen hexaose type 1 (GalNAc-LNFP I), blood group B antigen hexaose type 1 (Gal-LNFP I), lacto-N-fucopentaose II (LNFP II), lacto-N-fucopentaose III (LNFP III), lacto-N-fucopentaose V (LNFP V), lacto-N-difucohexaose I (LNDFH I), lacto-N-difucohexaose II (LNDFH II), lewis b-lewis x, monofucosyllacto-N-hexaose III (MFLNH III), difucosyllacto-N-hexaose (a) (DFLNH (a)), difucosyllacto-N-hexaose (DFLNH), trifucosyllacto-N-hexaose (TFLNH), lacto-N-neofucopentaose I (LNnFP I), lacto-N-neofucopentaose V (LNnFP V, LNFP VI), and lacto-N-neodifucohexaose (LNnDFH), more preferably selected from the group consisting of 2′FL, diFL, LNFP I, GalNAc-LNFP I, Gal-LNFP I, LNDFH I, lewis b-lewis x, DFLNH (a), TFLNH and LNnFP I, even more preferably selected from the group consisting of 2′FL, diFL, LNFP I and LNnFP I, even more preferably wherein the fucosylated mammalian milk oligosaccharide is 2′FL or diFL, most preferably wherein the fucosylated mammalian milk oligosaccharide is 2′FL.

A fucosylated oligosaccharide comprising lacto-N-biose (LNB) at its reducing end is preferably selected form the group consisting of 2′-fucosyllacto-N-biose (2′FLNB), 4-fucosyllacto-N-biose (4FLNB) and difucosyllacto-N-biose (diFLNB), more preferably wherein the fucosylated mammalian milk oligosaccharide is 2′FLNB or diFLNB, most preferably wherein the fucosylated mammalian milk oligosaccharide is 2′FLNB.

A fucosylated oligosaccharide comprising N-acetyllactosamine (LacNac) at its reducing end is preferably selected form the group consisting of 2′-fucosyl-N-acetyllactosamine (2′FlacNAc), difucosyl-N-acetyllactosamine (diFLacNAc) and 3-fucosyl-N-acetyllactosamine (3FlacNAc), more preferably wherein the fucosylated mammalian milk oligosaccharide is 2′FlacNAc or diFLacNAc, most preferably wherein the fucosylated mammalian milk oligosaccharide is 2′FlacNAc.

In a more preferred embodiment, the fucosylated MMO according to the disclosure is selected from a group consisting of 2′-fucosyllactose (2′FL), 3-fucosyllactose (3-FL), difucosyllactose (diFL), 2′-fucosyl-N-acetyllactosamine (2′FlacNAc), difucosyl-N-acetyllactosamine (diFLacNAc), 3-fucosyl-N-acetyllactosamine (3FlacNAc), 2′-fucosyllacto-N-biose (2′FLNB), 4-fucosyllacto-N-biose (4FLNB), difucosyllacto-N-biose (diFLNB), lacto-N-fucopentaose I (LNFP I), blood group A antigen hexaose type 1 (GalNAc-LNFP I), blood group B antigen hexaose type 1 (Gal-LNFP I), lacto-N-fucopentaose II (LNFP II), lacto-N-fucopentaose III (LNFP III), lacto-N-fucopentaose V (LNFP V), lacto-N-difucohexaose I (LNDFH I), lacto-N-difucohexaose II (LNDFH II), lewis b-lewis x, monofucosyllacto-N-hexaose III (MFLNH III), difucosyllacto-N-hexaose (a) (DFLNH (a)), difucosyllacto-N-hexaose (DFLNH), trifucosyllacto-N-hexaose (TFLNH), lacto-N-neofucopentaose I (LNnFP I), lacto-N-neofucopentaose V (LNnFP V, LNFP VI), and lacto-N-neodifucohexaose (LNnDFH). More preferably, the fucosylated MMO according to the disclosure is selected from the group consisting of 2′FL, diFL, LNFP I, GalNAc-LNFP I, Gal-LNFP I, LNDFH I, lewis b-lewis x, DFLNH (a), TFLNH, LNnFP I, 2′FLNB, diFLNB, 2′FlacNAc and diFLacNAc, even more preferably selected from the group consisting of 2′FL, diFL, LNFP I, LNnFP I, 2′FLNB, diFLNB, 2′FlacNAc and diFLacNAc, even more preferably selected from the group consisting of 2′FL, diFL, 2′FLNB, diFLNB, 2′FlacNAc and diFLacNAc, even more preferably selected from the group consisting of 2′FL, 2′FLNB and 2′FlacNAc, even more preferably wherein the fucosylated mammalian milk oligosaccharide is 2′FL or diFL, most preferably wherein the fucosylated mammalian milk oligosaccharide is 2′FL.

Aforementioned fucosylated mammalian milk oligosaccharides are commercially available and/or the production/purification of these oligosaccharides has been described and hence allows the skilled person to produce/obtain any of the fucosylated oligosaccharides accordingly. For example, (each reference is incorporated by reference):
2′FL: Zhou et al., 2021, ACS Synth. Biol. 10 (3): p. 447-458; Lu et al., 2021, ACS Synth. Biol. 10 (5): p. 923-938; WO2022/034073; Carbosynth (OF06739)
3-FL: Zhou et al., 2021; ACS Synth. Biol. 10 (3): p. 447-458, Lu et al., 2021, ACS Synth. Biol. 10 (5): p. 923-938; WO2022/034073; Carbosynth (OF05673)
diFL: WO2022/034073
2′FlacNAc: WO 2022/034075
3FlacNAc: WO 2022/034075
diFLacNAc: WO 2022/034075
2′FLNB: WO 2022/034075
4FLNB: WO 2022/034075
diFLNB: WO 2022/034075
LNFP I: Derya et al., 2020, J. Biotech. 319: p. 31-38; Carbosynth (OL05676)
GalNAc-LNFP I: WO 2022/034077
Gal-LNFP I: WO 2022/034077
LNFP II: Zeuner et al., 2018, ACS Synth. Biol. 10 (3): p. 447-458
LNFP III: Zeuner et al., 2018, ACS Synth. Biol. 10 (3): p. 447-458; Carbosynth (OL04212)
LNFP V: WO2020/115671; Carbosynth (OL06817)
LNDFH I: Huang et al., 2021, ACS Catal. 11 (5): p. 2631-2643; Carbosynth (OL01664)
LNDFH II: Yu et al., 2017, chem. Comm. 53 (80): p. 11012-11015; Huang et al., 2021, ACS Catal. 11 (5): p. 2631-2643; Carbosynth (OL06826)
MFLNH III: Carbosynth (OM05898)
DFLNH (a): Carbosynth (OD05375)
DFLNH: Carbosynth (OD06532)
TFLNH: Isosep (57/18-0010)
LNnFP I: Elicityl (GLY033-2-90%)
LNnFP V: Dumon et al., 2001, Glyconj. J. 18 (6): p. 465-474
LNnDFH: Dumon et al., 2001, Glyconj. J. 18 (6): p. 465-474

In an additional and/or alternative more preferred embodiment, the fucosylated MMO according to the disclosure (and optionally any, preferably all, further mammalian milk oligosaccharides, if also administered to the animal (it is also referred to the Section “Composition”)) has been isolated from a microbial cultivation or fermentation, cell culture, enzymatic reaction or chemical reaction. Alternatively, the fucosylated MMO of the disclosure (and optionally any, preferably all, further mammalian milk oligosaccharides, if also administered to the animal (it is also referred to the Section “Composition”)) has been isolated by e.g., chromatography or filtration technology from a natural source such as a human or animal milk, preferably animal milk. Isolation of the fucosylated MMO typically results in a solution containing the oligosaccharide. Such a solution can, for example, be obtained by a method comprising the steps of:
cultivating at least one cell as defined herein, preferably a single cell, which is capable to produce the fucosylated MMO, in a suitable cultivation medium to form a cultivation broth, preferably wherein the cell is metabolically engineered for the production of the fucosylated MMO, and
purifying the fucosylated MMO from the cultivation broth by:
clarifying the cultivation broth, and
removing salts and/or medium components form the clarified cultivation broth, and/or
concentrating the fucosylated oligosaccharide in the clarified cultivation broth,
thereby providing a solution comprising a purified oligosaccharide.

In the context of the disclosure, “clarifying the cultivation broth” refers to the removal of suspended particulates and contaminants, particularly cells, cell components, insoluble metabolites and debris produced by culturing the cell according to the disclosure. Clarification is preferably one or more of centrifugation, flocculation, decantation and/or filtration.

In the context of the disclosure, “removing salts and/or medium components form the clarified cultivation broth” refers to removing substantially all the proteins, as well as peptides, amino acids, RNA and DNA and any endotoxins and glycolipids from the clarified cultivation broth, preferably after it has been. In this step, proteins and related impurities can be removed from the saccharide in a conventional manner. Preferably, proteins, salts, by-products, color, endotoxins and other related impurities are removed from the oligosaccharide by ultrafiltration, nanofiltration, two-phase partitioning, reverse osmosis, microfiltration, activated charcoal or carbon treatment, treatment with non-ionic surfactants, enzymatic digestion, tangential flow high-performance filtration, tangential flow ultrafiltration, electrophoresis (e.g., using slab-polyacrylamide or sodium dodecyl sulphate-polyacrylamide gel electrophoresis (PAGE)), affinity chromatography (using affinity ligands including e.g., DEAE-Sepharose, poly-L-lysine and polymyxin-B, endotoxin-selective adsorber matrices), ion exchange chromatography (such as but not limited to cation exchange, anion exchange, mixed bed ion exchange, inside-out ligand attachment), hydrophobic interaction chromatography and/or gel filtration (i.e., size exclusion chromatography), particularly by chromatography, more particularly by ion exchange chromatography or hydrophobic interaction chromatography or ligand exchange chromatography. With the exception of size exclusion chromatography, proteins and related impurities are retained by a chromatography medium or a selected membrane, the oligosaccharide remains in the oligosaccharide containing clarified cultivation broth.

Further purification of the oligosaccharide may be accomplished, for example, by use of (activated) charcoal or carbon, nanofiltration, ultrafiltration, electrophoresis, enzymatic treatment or ion exchange to remove any remaining DNA, protein, LPS, endotoxins, or other impurity. Alcohols, such as ethanol, and aqueous alcohol mixtures can also be used. Another purification step is accomplished by crystallization, evaporation or precipitation of the product.

Several drying techniques are known to the skilled person, which can be used to obtain powder from a solution containing the oligosaccharide. Powder is preferably obtained by spray drying, freeze drying, spray freeze-drying, crystallization, lyophilization, band or belt drying, drum or roller drying, and/or agitated thin film drying, preferably by spray drying, drum or roller drying, or agitated thin film drying, more preferably by spray drying or agitated thin film drying, most preferably by spray drying, of a solution containing the saccharide. Preferably, the oligosaccharide according to the disclosure constitutes ≥70.0%, preferably ≥75.0%, more preferably ≥80.0, (w/w) of the powder. Preferably, the powder contains ≤15 wt. %, preferably ≤10 wt. %, more preferably ≤9 wt. %, more preferably ≤8 wt. %, more preferably ≤7 wt. %, even more preferably ≤5 wt. %, even more preferably ≤4 wt. % of liquid, even more preferably ≤3 wt. % of liquid, even more preferably ≤2 wt. % of liquid, most preferably ≤1 wt. %, preferably wherein the liquid is water.

In an additional and/or alternative more preferred embodiment, the fucosylated MMO according to the disclosure (and optionally any, preferably all, further mammalian milk oligosaccharides, if also administered to the animal (it is also referred to the Section “Composition”)) is provided as a powder according to the disclosure. Preferably, the powder is as defined earlier in the present Section.

In an additional and/or alternative more preferred embodiment, the fucosylated MMO according to the disclosure (and optionally any, preferably all, further mammalian milk oligosaccharides, if also administered to the animal (it is also referred to the Section “Composition”)) has been produced, preferably in vitro and/or ex vivo, by a cell, preferably a single cell, wherein the cell is preferably chosen from the group consisting of a microorganism, a plant cell, an animal cell and a protozoan cell. In other words, the saccharide of the disclosure has been produced by an in vitro and/or ex vivo culture of cells, wherein the cells are preferably chosen from the group consisting of a microorganism, a plant cell, an animal cell or a protozoan cell.

Preferably, the cell is a microorganism. Preferably, the microorganism is selected from a group consisting of a bacterium, a yeast and a fungus. Further, it is preferred that the cell is genetically engineered for the production of the fucosylated MMO according to the disclosure. Throughout the description and claims, the terms “has been isolated” and “is obtained” are interchangeably used, unless specifically stated otherwise.

The latter bacterium preferably belongs to the phylum of the Proteobacteria or the phylum of the Firmicutes or the phylum of the Cyanobacteria or the phylum Deinococcus-Thermus, preferably belongs to the phylum of the Proteobacteria. The latter bacterium belonging to the phylum Proteobacteria belongs preferably to the family Enterobacteriaceae, preferably to the species Escherichia coli. The latter bacterium preferably relates to any strain belonging to the species E. coli such as but not limited to E. coli B, E. coli C, E. coli W, E. coli K12, E. coli Nissle. More specifically, the latter term relates to cultivated E. coli strains-designated as E. coli K12 strains-which are well-adapted to the laboratory environment, and, unlike wild type strains, have lost their ability to thrive in the intestine. Well-known examples of the E. coli K12 strains are K12 Wild type, W3110, MG1655, M182, MC1000, MC1060, MC1061, MC4100, JM101, NZN111 and AA200. Hence, preferably, the disclosure specifically relates to a mutated and/or transformed E. coli strain as indicated above wherein the E. coli strain is a K12 strain. More specifically, the disclosure relates to a mutated and/or transformed E. coli strain as indicated above wherein the K12 strain is E. coli MG1655. The latter bacterium belonging to the phylum Firmicutes belongs preferably to the Bacilli, preferably from the species Bacillus, such as Bacillus subtilis or, B. amyloliquefaciens. The latter Bacterium belonging to the phylum Actinobacteria, preferably belonging to the family of the Corynebacteriaceae, with members Corynebacterium glutamicum or C. afermentans, or belonging to the family of the Streptomycetaceae with members Streptomyces griseus or S. fradiae. The latter yeast preferably belongs to the phylum of the Ascomycota or the phylum of the Basidiomycota or the phylum of the Deuteromycota or the phylum of the Zygomycetes. The latter yeast belongs preferably to the genus Saccharomyces (with members like e.g., Saccharomyces cerevisiae, S. bayanus, S. boulardii), Pichia (with members like e.g., Pichia pastoris, P. anomala, P. kluyveri), Komagataella, Hansunella, Kluyveromyces (with members like e.g., Kluyveromyces lactis, K. marxianus, K. thermotolerans), Yarrowia (like e.g., Yarrowia lipolytica), Eremothecium, Zygosaccharomyces, Starmerella (like e.g., Starmerella bombicola) or Debaromyces. The latter yeast is preferably selected from Pichia pastoris, Yarrowia lipolitica, Saccharomyces cerevisiae and Kluyveromyces lactis. The latter fungus belongs preferably to the genus Rhizopus, Dictyostelium, Penicillium, Mucor or Aspergillus. “Plant cells” includes cells of flowering and non-flowering plants, as well as algal cells, for example, Chlamydomonas, Chlorella, etc. Preferably, the plant cell is a tobacco, alfalfa, rice, cotton, rapeseed, tomato, corn, maize or soybean cell. The latter animal cell is preferably derived from non-human mammals (e.g., cattle, buffalo, pig, sheep, mouse, rat), birds (e.g., chicken, duck, ostrich, turkey, pheasant), fish (e.g., swordfish, salmon, tuna, sea bass, trout, catfish), invertebrates (e.g., lobster, crab, shrimp, clams, oyster, mussel, sea urchin), reptiles (e.g., snake, alligator, turtle), amphibians (e.g., frogs) or insects (e.g., fly, nematode) or is a genetically modified cell line derived from human cells excluding embryonic stem cells. Both human and non-human mammalian cells are preferably chosen from the group comprising an epithelial cell like e.g., a mammary epithelial cell, mammary myoepithelial cell, mammary progenitor cell, an embryonic kidney cell (e.g., HEK293 or HEK 293T cell), a fibroblast cell, a COS cell, a Chinese hamster ovary (CHO) cell, a murine myeloma cell like e.g., an N20, SP2/0 or YB2/0 cell, an NIH-3T3 cell, a non-mammary adult stem cell or derivatives thereof such as described in WO21067641, preferably mesenchymal stem cell or derivates thereof as described in WO21067641. The insect cell is preferably derived from Spodoptera frugiperda like e.g., Sf9 or Sf21 cells, Bombyx mori, Mamestra brassicae, Trichoplusia ni like e.g., BTI-TN-5B1-4 cells or Drosophila melanogaster like e.g., Drosophila S2 cells. The latter protozoan cell preferably is a Leishmania tarentolae cell.

In an additional and/or alternative more preferred embodiment, the fucosylated MMO of the disclosure has been produced, preferably in vitro and/or ex vivo, by a mammary epithelial cell, mammary myoepithelial cell and/or mammary progenitor cell, preferably wherein the cell is generated from non-mammary adult stem cells, more preferably wherein the cell is generated from mesenchymal stem cells. Such cells are well-known to the skilled person, it is in this regard referred to, for example, WO2021/067641 and WO2021/242866 (mammary epithelial cells derived from non-mammary adult stem cells, preferably from mesenchymal stem cells) and WO2021/142241 (mammary epithelial cells, mammary myoepithelial cells, mammary progenitor cells).

In an even more preferred embodiment, the fucosylated MMO of the disclosure (and optionally any, preferably all, further mammalian milk oligosaccharides, if also administered to the animal (it is also referred to the Section “Composition”)) has been produced, preferably in vitro and/or ex vivo, by a microorganism cell, preferably the microorganism is a bacterium, a yeast or a fungus, more preferably the microorganism is a bacterium or a yeast, even more preferably the microorganism is a bacterium, most preferably the microorganism is Escherichia coli.

In the context of the disclosure, it is also within the scope of the disclosure that two or more different cells (preferably as defined herein), produce the fucosyalted MMO according to the disclosure. It is however preferred that a single cell produces the oligosaccharide, i.e., a single culture of the cell produces the oligosaccharide of the disclosure.

In an additional and/or alternative embodiment, the fucosylated MMO according to the disclosure (and optionally any, preferably all, further mammalian milk oligosaccharides, if also administered to the animal (it is also referred to the Section “Composition”)) is linked, preferably chemically linked, to a carrier for delivery of the MMO. Preferably, the carrier is a ceramide-based carrier or a polypeptide-based carrier, more preferably the carrier is a ceramide-based carrier. Preferably, the polypeptide-based carrier is epsilon-polylysine, alfa-polylysine, poly(aspartic acid), polyglutamic acid or polyornithine. These carriers are commercially available (e.g., Sigma-Aldrich, Carbosynth). The ceramide-based carrier is preferably selected from a group consisting of d18:1/16:0, t18:0-16:0, t18:0-h16:0, t18:0-h22:0 and t18:0-h24:0. These ceramide carriers are commercially available and well-known to the skilled person and are, for example, described in WO2010/037785, which is incorporated by reference. d18:1/16:0 is also known as C16 ceramide and N-palmitoylsphingosine and therefore interchangeable used herein. t18:0-16:0 is also known as C16 phytoceramide and N-hexadecanoyl phytosphingosine and therefore interchangeable used herein. t18:0-h16:0, t18:0-h22:0 and t18:0-h24:0 are glycosylinositolphosphoceramides (GIPCs). As known to the skilled person, “d” and “t” refer to the hydroxylation state of the whole ceramide or long-chain base moiety (d is 2 groups, t is 3 groups), whereas “h” denotes a hydroxylation of the fatty acyl group.

The fucosylated mammalian milk oligosaccharide and optionally any, preferably all, further mammalian milk oligosaccharides, if also administered to the animal., is preferably encapsulated (i.e., forming an encapsulate). This is particularly advantageous to protect the oligosaccharide(s) from conditions relating to the manufacturing or storing of a composition according to the disclosure, especially when the composition is a nutritional composition (it is referred to the Section “Composition”). Such conditions typically entail a high pressure (e.g., a pressure of above 10 bar during extrusion), a high temperature (e.g., a temperature above 100° C. during extrusion and baking), a pH lower than 5, a pH higher than 8, and/or a high moisture content (e.g., wet extrusion). Moreover, other molecules within a composition, especially a nutritional composition, such as proteins could chemically interact with the fucosylated MMO (and any other further MMO if present) especially under the harsh conditions during manufacturing of a nutritional composition. Hence, throughout the disclosure and claims, the “fucosylated MMO” is preferably replaced with “encapsulated fucosylated MMO”. If any further MMO is present (it is also referred to the Section “Composition”), than it is preferred that all MMO's are encapsulated, preferably all MMO's are co-encapsulated.

The encapsulated is particularly advantageous when the composition according to the disclosure is a nutritional composition (it is referred to the Section “Composition) which is manufactured by extrusion (dry or wet, preferably wet).

It is preferred that the encapsulate is of the core-shell type, i.e., carrier material forms a shell around the active agent (i.e., the MMO ('s) according to the disclosure), preferably wherein the core has a median diameter (D(v,0.5)) of 125-250 μm, preferably 150-250 μm, more preferably 165-250 μm, most preferably 165-225 μm, and wherein the encapsulate has a median diameter (D(v,0.5)) which is 20-100 μm, preferably 20-75 μm, more preferably 35-75 μm, longer than the median diameter of the core.

The carrier material preferably comprises a hot melt material, more preferably an oil and/or a wax, even more preferably a hydrogenated oil and/or a wax, optionally further comprising starch, preferably wherein the starch constitutes less than 15% (w/w) of the carrier material, more preferably the starch constitutes less than 10% (w/w) of the carrier material.

The oil is preferably selected from the group consisting of palm oil, sunflower oil, soybean oil, rapeseed oil, coconut oil, babassu oil, palm kernel oil, maize oil, sesame oil and cottonseed oil; more preferably the oil is selected from the group consisting of palm oil, sunflower oil, soybean oil and rapeseed oil; even more preferably the oil is palm oil, most preferably the oil is hydrogenated palm oil.

The wax is preferably selected from the group consisting of Candelilla wax, Carnauba wax, beeswax, rice bran wax, paraffin wax, jojoba wax, microcrystalline wax and Japan wax; more preferably the wax is Candelilla wax or Carnauba wax; most preferably the wax is Candelilla wax.

It is further preferred that the fucosylated MMO or all MMO's present in the encapsulate constitute 5-50% (w/w), preferably 10-40% (w/w), more preferably 20-40% (w/w), even more preferably 20-35% (w/w), of the total weight of the encapsulate.

Throughout the disclosure and claims, it is particularly preferred that the fucosylated MMO according to the disclosure and optionally any, preferably all, further mammalian milk oligosaccharides, if also administered to the animal, is part of a composition, preferably a composition according to the Section “Composition”.

### Composition

In a preferred embodiment of the first aspect, the disclosure provides a composition comprising a fucosylated mammalian milk oligosaccharide (MMO), wherein the composition is for use in a method for preventing and/or treating shipping fever in an animal., preferably a farm animal or domesticated animal, more preferably a farm animal.

The fucosylated MMO is as described in the Section “oligosaccharide”. The shipping fever is as described in the Section “Shipping fever”. The method for preventing and/or treating is as described in the Section “Method for preventing and/or treating”. The animal is as described in the Section “Subject”.

In a preferred embodiment, the fucosylated mammalian milk oligosaccharide (MMO) is present at 0.001-1.000 wt. %, preferably 0.001-0.750 wt. %, more preferably 0.001-0.500 wt. %, even more preferably 0.001-0.400 wt. %, even more preferably 0.005-0.400 wt. %, even more preferably 0.005-0.300 wt. %, even more preferably 0.005-0.250 wt. %, most preferably 0.005-0.100 wt. %, of the total weight of the composition. Throughout the disclosure and claims, unless specifically stated otherwise, the term “wt. %” means weight by weight (i.e., w/w). If the composition is, for example, a solid composition, then 1.0 wt. % means 1.0 g fucosylated MMO per 100.0 gram of the solid composition. If the composition is, for example, a liquid composition, then 1.0 wt. % means 1.0 g fucosylated MMO per 100.0 gram of the liquid composition.

Throughout the disclosure and claims, if the composition is a milk replacer (e.g., calf milk replacer) then the wt. % as recited throughout the disclosure and claims, refers to the drywt. %. If the composition is a (calf) milk replacer, then, for example, aforementioned paragraph should be read as: “In a preferred embodiment, the fucosylated mammalian milk oligosaccharide (MMO) is present at 0.001-1.000 drywt. %, preferably 0.001-0.750 drywt. %, more preferably 0.001-0.500 drywt. %, even more preferably 0.001-0.400 drywt. %, even more preferably 0.005-0.400 drywt. %, even more preferably 0.005-0.300 drywt. %, even more preferably 0.005-0.250 drywt. %, most preferably 0.005-0.100 drywt. %.”

Further, a milk replacer (e.g., calf milk replacer) is often administered to e.g., a calf in a liquid form. Hence, in this context, it is preferred that a liquid milk replacer comprises 10.0-30.0% (w/v), more preferably 12.0-28.0% (w/v), milk replacer powder. Here, it is preferred that the milk replacer powder is constituted in water to obtain the liquid milk replacer. Throughout the disclosure and claims, the term “% w/v)” refers to the weight of the dissolved substance in gram (e.g., milk replacer powder) per 100 milliliters of the final solution (e.g., liquid milk replacer). If the milk replacer is a calf milk replacer, then it is particularly preferred that a liquid calf milk replacer comprises 10.0-20.0% (w/v), more preferably 12.0-20.0% (w/v), even more preferably 12.0-18.0% (w/v), even more preferably 14.0-18.0% (w/v), most preferably 15.0% (w/v), calf milk replacer powder. It is preferred that the calf milk replacer powder is constituted in water to obtain the liquid calf milk replacer. If the milk replacer is a milk replacer for a piglet, then it is particularly preferred that a liquid milk replacer comprises 10.0-30.0% (w/v), more preferably 15.0-30.0% (w/v), even more preferably 20.0-30.0% (w/v), even more preferably 22.0-28.0% (w/v), most preferably 25.0% (w/v), milk replacer powder. It is preferred that the milk replacer powder is constituted in water to obtain the liquid milk replacer.

In a more preferred embodiment, the fucosylated MMO is present at 0.001-0.250 wt. %, preferably 0.001-0.200 wt. %, more preferably 0.001-0.100 wt. %, even more preferably 0.001-0.020 wt. %, even more preferably 0.001-0.010 wt. %, even more preferably 0.002-0.010 wt. %, even more preferably 0.003-0.010 wt. %, even more preferably 0.003-0.0075 wt. %, most preferably 0.003-0.006 wt. %, of the total weight of the composition. This embodiment is, for example, particularly preferred if the animal is a bovine or a horse, more preferably wherein the animal is a calf or a foal, most preferably wherein the animal is a calf.

In an alternative more preferred embodiment, the fucosylated MMO is present at 0.010-1.000 wt. %, preferably 0.010-0.750 wt. %, more preferably 0.010-0.500 wt. %, even more preferably 0.010-0.400 wt. %, even more preferably 0.050-0.400 wt. %, even more preferably 0.100-0.400 wt. %, most preferably 0.100-0.300 wt. %, of the total weight of the composition. This embodiment is, for example, particularly preferred if the animal is a pig, a sheep, a goat or a camelid (preferably llama or alpaca), more preferably wherein the animal is a piglet, a lamb, a kid or a cria, even more preferably wherein the animal is a piglet, a lamb or a kid, most preferably wherein the animal is a piglet.

In a preferred embodiment of the disclosure, the composition further comprises a second mammalian milk oligosaccharide (MMO), preferably wherein the second mammalian milk oligosaccharide is a sialylated MMO, more preferably wherein the second mammalian milk oligosaccharide is a sialylated MMO comprising a lactose, a lacto-N-biose (LNB) or N-acetyllactosamine (LacNAc) at its reducing end, even more preferably the sialylated MMO comprises lactose or LacNAc at is reducing end, most preferably the sialylated MMO comprises lactose at its reducing end.

Preferably, the second MMO consists of 3-8, more preferably 3-7, even more preferably 3-6, most preferably 3-5, monosaccharides.

A sialylated MMO comprising lactose at its reducing end is preferably selected from the group consisting of 3′-sialyllactose (3′SL), 6′-sialyllactose (6′SL), 3,6-disialyllactose, 6,6′-disialyllactose, 8,3-disialyllactose, 3′S-2′FL, 6′S-2′FL, 3′S-3-FL, 6′S-3-FL, LST a, LST b, LST c and LST d; more preferably selected from the group consisting of 3′SL, 6′SL, 3,6-disialyllactose, 6,6′-disialyllactose, 8,3-disialyllactose, LST a, LST b, LST c and LST d; even more preferably selected from the group consisting of 3′SL, 6′SL, LST a, LST b, LST c and LST d; most preferably wherein the sialylated MMO is 3′-sialyllactose (3′SL) or 6′-sialyllactose (6′SL).

A sialylated MMO comprising lacto-N-biose (LNB) at its reducing end is preferably selected form the group consisting of 3′-sialyllacto-N-biose (3′SLNB), 6′-sialyllacto-N-biose (6′SLNB) and monofucosylmonosialyllacto-N-octaose (sialyl Lewis a); more preferably the sialylated MMO is 3′SLNB or 6′SLNB.

A sialylated MMO comprising N-acetyllactosamine (LacNac) at its reducing end is preferably selected form the group consisting of 3′-sialyllactosamine (3′SLacNAc), 6′-sialyllactosamine (6′SLacNAc) and sialyl Lewis x; more preferably the sialylated MMO is 3′SLacNAc or 6′SLacNAc.

When a second MMO, preferably a sialylated MMO, is present in the composition, then it is a preferred embodiment that the amount of the second MMO in the composition is 0.1-30.0, preferably 0.1-25.0, more preferably 0.1-20.0, even more preferably 0.1-15.0, even more preferably 0.2-15.0, even more preferably 0.3-15.0, even more preferably 0.4-15.0, most preferably 0.5-15.0, times the amount of the fucosylated MMO in the composition. It is a more preferred embodiment that the amount of the fucosylated MMO in the composition is lower than the amount of the second MMO, preferably the amount of the fucosylated MMO is 1.1 to 15.0, more preferably 1.5 to 15.0, even more preferably 2.5 to 15.0, even more preferably 5.0 to 15.0, even more preferably 5.0 to 12.5, most preferably 5.0 to 10.0, times lower than the amount of the second MMO in the composition. It is an even more preferred embodiment that, when the second MMO is a sialylated MMO, that the amount of the fucosylated MMO in the composition is lower than the amount of the second MMO, preferably the amount of the fucosylated MMO is 1.1 to 10.0, preferably 1.5-7.5, more preferably 2.5-7.5, most preferably 4.0-7.0, times lower than the amount of the second MMO in the composition.

In an additional preferred embodiment, the composition further comprises a third mammalian milk oligosaccharide (MMO), preferably wherein the third mammalian milk oligosaccharide is a sialylated MMO, more preferably wherein the third mammalian milk oligosaccharide is a sialylated MMO comprising a lactose, a lacto-N-biose (LNB) or N-acetyllactosamine (LacNAc) at its reducing end, even more preferably the sialylated MMO comprises lactose or LacNAc at is reducing end, most preferably the sialylated MMO comprises lactose at its reducing end.

Preferably, the third MMO consists of 3-8, more preferably 3-7, even more preferably 3-6, most preferably 3-5, monosaccharides.

A sialylated MMO comprising lactose at its reducing end is preferably selected from the group consisting of 3′-sialyllactose (3′SL), 6′-sialyllactose (6′SL), 3,6-disialyllactose, 6,6′-disialyllactose, 8,3-disialyllactose, 3′S-2′FL, 6′S-2′FL, 3′S-3-FL, 6′S-3-FL, LST a, LST b, LST c and LST d; more preferably selected from the group consisting of 3′SL, 6′SL, 3,6-disialyllactose, 6,6′-disialyllactose, 8,3-disialyllactose, LST a, LST b, LST c and LST d; even more preferably selected from the group consisting of 3′SL, 6′SL, LST a, LST b, LST c and LST d; most preferably wherein the sialylated MMO is 6′-sialyllactose (6′SL) or 3′-sialyllactose (3′SL).

A sialylated MMO comprising lacto-N-biose (LNB) at its reducing end is preferably selected form the group consisting of 3′-sialyllacto-N-biose (3′SLNB), 6′-sialyllacto-N-biose (6′SLNB) and monofucosylmonosialyllacto-N-octaose (sialyl Lewis a); more preferably the sialylated MMO is 3′SLNB or 6′SLNB.

A sialylated MMO comprising N-acetyllactosamine (LacNac) at its reducing end is preferably selected form the group consisting of 3′-sialyllactosamine (3′SLacNAc), 6′-sialyllactosamine (6′SLacNAc) and sialyl Lewis x; more preferably the sialylated MMO is 3′SLacNAc or 6′SLacNAc.

When a third MMO, preferably a sialylated MMO, is present in the composition, then it is a preferred embodiment that the amount of the third MMO in the composition is 0.1-30.0, preferably 0.1-25.0, more preferably 0.1-20.0, even more preferably 0.1-15.0, even more preferably 0.2-15.0, even more preferably 0.3-15.0, even more preferably 0.4-15.0, most preferably 0.5-15.0, times the amount of the fucosylated MMO in the composition. It is a more preferred embodiment that the amount of the fucosylated MMO in the composition is lower than the amount of the third MMO, preferably the amount of the fucosylated MMO is 1.1 to 15.0, more preferably 1.5 to 15.0, even more preferably 2.5 to 15.0, even more preferably 5.0 to 15.0, even more preferably 5.0 to 12.5, most preferably 5.0 to 10.0, times lower than the amount of the third MMO in the composition. It is an even more preferred embodiment that, when the third MMO is a sialylated MMO, that the amount of the fucosylated MMO in the composition is lower than the amount of the third MMO, preferably the amount of the fucosylated MMO is 1.1 to 10.0, preferably 1.5-7.5, more preferably 1.5-5.0, most preferably 2.5-5.0, times lower than the amount of the third MMO in the composition.

When a second MMO (preferably a sialylated MMO, i.e., a first sialylated MMO) and a third MMO (preferably a sialylated MMO, i.e., a second sialylated MMO) are present in the composition, then it is a preferred embodiment that the ratio between the amount of the second MMO and the amount of the third MMO in the composition is similar to this ratio as found in the mother's milk of the same species. Throughout the disclosure and claims, unless specifically stated otherwise, a “similar” ratio preferably refers to a ratio, which is identical or falls in the range of ratio +/−20% of the ratio, preferably ratio +/−10% of the ratio. When the animal is a bovine, the second MMO is a sialylated MMO and the third MMO is a sialylated MMO, the ratio is preferably 1.1 to 6.0, more preferably 1.1 to 5.0, even more preferably 1.25 to 4.0, even more preferably 1.25 to 3.0, even more preferably 1.5 to 3.0, most preferably 1.5 to 2.5. When the animal is a horse, the second MMO is a sialylated MMO and the third MMO is a sialylated MMO, the ratio is preferably 1.1 to 15.0, more preferably 1.1 to 12.5, even more preferably 1.1 to 10.0, even more preferably 1.5 to 10.0, even more preferably 2.5 to 10, even more preferably 5.0 to 10.0, even more preferably 7.5 to 10.0, most preferably 8.0 to 10.0. When the animal is a pig, the second MMO is a sialylated MMO and the third MMO is a sialylated MMO, the ratio is preferably 7.5-40.0, more preferably 7.5-35.0, even more preferably 10.0-35.0, even more preferably 15.0-35.0, even more preferably 20.0-35.0, most preferably 25.0-35.0. When the animal is a goat, the second MMO is a sialylated MMO and the third MMO is a sialylated MMO, the ratio is preferably 0.25-5.0, more preferably 0.25-3.0, even more preferably 0.25-2.0, even more preferably 0.25-1.5, most preferably 0.5-1.5. Further, it is a more preferred embodiment that the amount of the second MMO in the composition is higher than the amount of the third MMO, preferably the amount of the second MMO is 1.1 to 40.0, more preferably 1.1 to 35.0, most preferably 1.5 to 35.0, times the amount of the third MMO in the composition. Further, it is an even more preferred embodiment that the amount of the second MMO, when the second MMO is a sialylated MMO, in the composition is higher than the amount of the third MMO, when the third MMO is a sialylated MMO, preferably the amount of the second MMO is 1.1 to 6.0, more preferably 1.1 to 5.0, even more preferably 1.25 to 4.0, even more preferably 1.25-3.0, even more preferably 1.5 to 3.0, most preferably 1.5-2.5, times higher than the amount of the third MMO in the composition.

In a more preferred embodiment, the composition further comprises a second mammalian milk oligosaccharide (MMO), which is a sialylated MMO selected from the group consisting of 3′-sialyllactose (3′SL), 6′-sialyllactose (6′SL), 3,6-disialyllactose, 6,6′-disialyllactose, 8,3-disialyllactose, 3′S-2′FL, 6′S-2′FL, 3′S-3-FL, 6′S-3-FL, LST a, LST b, LST c, LST d. 3′-sialyllacto-N-biose (3′SLNB), 6′-sialyllacto-N-biose (6′SLNB), monofucosylmonosialyllacto-N-octaose (sialyl Lewis a), 3′-sialyllactosamine (3′SLacNAc), 6′-sialyllactosamine (6′SLacNAc) and sialyl Lewis x. More preferably the second MMO is selected from the group consisting of 3′SL, 6′SL, LST a, LST b, LST c, LST d, 3′SLNB, 6′SLNB, sialyl Lewis a, 3′SLacNAc, 6′SLacNAc and sialyl Lewis x. Even more preferably the second MMO is selected from the group consisting of 3′SL, 6′SL, 3′SLNB, 6′SLNB, 3′SLacNAc and 6′SLacNAc. Most preferably the second MMO is 3′-sialyllactose (3′SL) or 6′-sialyllactose (6′SL).

In an additional more preferred embodiment, the composition further comprises a third mammalian milk oligosaccharide (MMO), which is a sialylated MMO selected from the group consisting of 3′-sialyllactose (3′SL), 6′-sialyllactose (6′SL), 3,6-disialyllactose, 6,6′-disialyllactose, 8,3-disialyllactose, 3′S-2′FL, 6′S-2′FL, 3′S-3-FL, 6′S-3-FL, LST a, LST b, LST c, LST d, 3′-sialyllacto-N-biose (3′SLNB), 6′-sialyllacto-N-biose (6′SLNB), monofucosylmonosialyllacto-N-octaose (sialyl Lewis a), 3′-sialyllactosamine (3′SLacNAc), 6′-sialyllactosamine (6′SLacNAc) and sialyl Lewis x. More preferably the third MMO is selected from the group consisting of 3′SL, 6′SL, LST a, LST b, LST c, LST d, 3′SLNB, 6′SLNB, sialyl Lewis a, 3′SLacNAc, 6′SLacNAc and sialyl Lewis x. Even more preferably the third MMO is selected from the group consisting of 3′SL, 6′SL, 3′SLNB, 6′SLNB, 3′SLacNAc and 6′SLacNAc. Most preferably the third MMO is 6′-sialyllactose (6′SL) or 3′-sialyllactose (3′SL).

In the context of the disclosure, the second mammalian milk oligosaccharide and the third mammalian milk oligosaccharide preferably do not contain N-acetyllactosamine.

Surprisingly, it was found that a fucosylated MMO according to the disclosure, preferably a composition comprising a fucosylated MMO according to the disclosure, is sufficient to obtain an effective therapeutic for preventing and/or treating shipping fever as described herein. The presence of other (oligo) saccharides is not required to obtain the therapeutic effect. While the composition according to the disclosure can include additional (oligo) saccharides, the disclosure relates to the fact that only the fucosylated MMO according to the disclosure is required for achieving an efficient therapeutic effect as described herein.

Itis a preferred embodiment that the composition according to the disclosure does not comprise lacto-N-tetraose (LNT).

In an additional and/or alternative preferred embodiment, the composition according to the disclosure does not comprise lacto-N-neotetraose (LNnT).

In an additional and/or alternative preferred embodiment, the composition according to the disclosure does not comprise N-acetyllactosamine.

In an additional and/or alternative preferred embodiment, the composition according to the disclosure does not comprise an oligosaccharide containing N-acetyllactosamine, preferably does not comprise a saccharide containing N-acetyllactosamine.

In an additional and/or alternative preferred embodiment, the composition according to the disclosure does not comprise 3′sialyllactose.

In an additional and/or alternative preferred embodiment, the composition according to the disclosure does not comprise 6′sialyllactose.

More preferably, the composition according to the disclosure does not comprise a sialylated mammalian milk oligosaccharide, even more preferably does not comprise a sialylated oligosaccharide, most preferably does not comprise a sialylated saccharide.

In an additional and/or alternative preferred embodiment, the composition according to the disclosure does not comprise a fructo-oligosaccharide (FOS).

In an additional and/or alternative preferred embodiment, the composition according to the disclosure does not comprise a galactose-oligosaccharide (GOS).

In an additional and/or alternative preferred embodiment, the composition according to the disclosure does not comprise an isomaltooligosaccharide.

In a more preferred embodiment, the composition according to the disclosure comprises the fucosylated MMO as described herein, optionally a second MMO as described herein and optionally a third MMO as described herein, and no additional MMOs (preferably no additional oligosaccharides, more preferably no additional saccharides).

In an even more preferred embodiment, the composition according to the disclosure comprises the fucosylated MMO as described herein, and no additional MMOs (preferably no additional oligosaccharides, more preferably no additional saccharides).

In an even more preferred embodiment, the composition according to the disclosure does not comprise a neutral non-fucosylated MMO.

In an even more preferred embodiment, the composition according to the disclosure comprises only one mammalian milk oligosaccharide, i.e., the fucosylated MMO as described herein.

In a preferred embodiment, the composition according to the disclosure is not mother's milk.

In an additional and/or alternative preferred embodiment, the composition according to the disclosure is a synthetic composition.

In the context of the disclosure, a “synthetic composition” or a “synthetic nutritional composition” refers to a composition that is artificially prepared and preferably refers to a composition comprising at least one component that is produced ex vivo, either chemically and/or biologically, e.g., by way of chemical reaction, enzymatic reaction or recombinantly, or purified by humans. It is preferred that a synthetic composition of the disclosure is not identical with a naturally occurring composition.

In this context, it is preferred that the fucosylated MMO as described herein has been isolated from a microbial cultivation or fermentation, cell culture, enzymatic reaction or chemical reaction. Or has been isolated from a natural source (it is referred to the Section “Oligosaccharide”). It is more preferred that the fucosylated MMO as described herein has been produced in vitro and/or ex vivo by a cell (it is referred to the Section “Oligosaccharide”).

In an additional and/or alternative preferred embodiment, the composition according to the disclosure further comprises a microbial strain that does not use, preferably is not able to use, the fucosylated MMO as a carbon source.

In an alternative preferred embodiment, the composition according to the disclosure does not comprise a microbial strain. Examples of such a strain is Bifidobacterium, Lactobacillus and Saccharomyces boulardii.

In a preferred embodiment, the composition according to the disclosure is a pharmaceutical composition, optionally further comprising a pharmaceutically acceptable carrier, filler, preservative, solubilizer, diluent, excipient, salt, adjuvant and/or solvent.

In an additional and/or alternative preferred embodiment, the composition is a nutritional composition, optionally further comprising a feed ingredient and/or a food ingredient, wherein the feed/food ingredient is preferably chosen from the group consisting of:
a lipid, preferably one or more selected from the group consisting of an oil, fat, ester, monoglyceride, diglyceride, triglyceride and free fatty acid;
a vitamin, preferably one or more selected from the group consisting of vitamin A, vitamin B, vitamin C, vitamin D, vitamin E and vitamin H, or a derivate thereof;
an amino acid compound;
a trace element;
a mineral, preferably one or more selected from the group consisting of calcium, phosphorus, magnesium, iron, zinc, manganese, copper, sodium, potassium, molybdenum, chromium, selenium and chloride;
an antioxidant;
a prebiotic agent, preferably one or more selected from the group consisting of GOS (galacto-oligosaccharide), FOS (fructo-oligosaccharide), inulin and resistant starch;
a carbohydrate;
an antimicrobial agent; and/or
a protein.

Generally, any source of protein may be used so long as it is suitable for nutritional compositions and is otherwise compatible with any other selected ingredients or features in the nutritional composition. Non-limiting examples of suitable proteins (and sources thereof) suitable for use in the nutritional composition according to the disclosure include, but are not limited to, intact, hydrolyzed, or partially hydrolyzed protein, which may be derived from any known or otherwise suitable source such as milk (e.g., casein, whey), animal (e.g., meat, fish), cereal (e.g., rice, corn, wheat), vegetable (e.g., soy, pea, potato, bean), and combinations thereof. The protein may also include a mixture of amino acids (often described as free amino acids) known for use in nutritional products or a combination of such amino acids with the intact, hydrolyzed, or partially hydrolyzed proteins described herein. The amino acids may be naturally occurring or synthetic amino acids. More particular examples of suitable protein (or sources thereof) used in a nutritional composition according to the disclosure include, but are not limited to, whole cow's milk, partially or completely defatted milk, milk protein concentrates, milk protein isolates, nonfat dry milk, condensed skim milk, whey protein concentrates, whey protein isolates, acid caseins, sodium caseinates, calcium caseinates, potassium caseinates, legume protein, soy protein concentrates, soy protein isolates, pea protein concentrates, pea protein isolates, collagen proteins, potato proteins, rice proteins, wheat proteins, canola proteins, quinoa, insect proteins, earthworm proteins, fungal (e.g., mushroom) proteins, hydrolyzed yeast, gelatin, bovine colostrum, human colostrum, glycol macropeptides, mycoproteins, proteins expressed by microorganisms (e.g., bacteria and algae), and combinations thereof. A nutritional composition according to the disclosure may include any individual source of protein or combination of the various sources of protein grouped above. In addition, the proteins for use herein can also include, or be entirely or partially replaced by, free amino acids known for use in nutritional products, non-limiting examples of which include L-tryptophan, L-glutamine, L-tyrosine, L-methionine, L-cysteine, taurine, L-arginine, L-carnitine, and combinations thereof.

The carbohydrate or source of carbohydrate suitable for use in a nutritional composition according to the disclosure may be simple, complex, or variations or combinations thereof. Generally, the carbohydrate may include any carbohydrate or carbohydrate source that is suitable for use nutritional compositions and is otherwise compatible with any other selected ingredients or features in the nutritional composition. Non-limiting examples of carbohydrates suitable for use in the nutritional composition according to the disclosure, but are not limited to, polydextrose, maltodextrin; hydrolyzed or modified starch or cornstarch; glucose polymers; corn syrup; corn syrup solids; sucrose; glucose; fructose; lactose; high fructose corn syrup; honey; sugar alcohols (e.g., maltitol, erythritol, sorbitol); isomaltulose; sucromalt; pullulan; potato starch; and other slowly-digested carbohydrates; dietary fibers including, but not limited to, fructooligosaccharides (FOS), galactooligosaccharides (GOS), oat fiber, soy fiber, gum arabic, sodium carboxymethylcellulose, methylcellulose, guar gum, gellan gum, locust bean gum, konjac flour, hydroxypropyl methylcellulose, tragacanth gum, karaya gum, gum acacia, chitosan, arabinogalactans, glucomannan, xanthan gum, alginate, pectin, low methoxy pectin, high methoxy pectin, cereal beta-glucans (e.g., oat beta-glucan, barley beta-glucan), carrageenan and psyllium, soluble and insoluble fibers derived from fruits or vegetables; other resistant starches; and combinations thereof. A nutritional composition according to the disclosure may include any individual source of carbohydrate or combination of the various sources of carbohydrate grouped above.

The fat or source of fat suitable for use in a nutritional composition according to the disclosure may be derived from various sources including, but not limited to, plants, animals, and combinations thereof. Generally, the fat may include any fat or fat source that is suitable for use in a nutritional composition according to the disclosure and is otherwise compatible with any other selected ingredients or features in the nutritional composition. Non-limiting examples of suitable fat (or sources thereof) for use in a nutritional composition according to the disclosure include coconut oil, fractionated coconut oil, soy oil, high oleic soy oil, corn oil, olive oil, safflower oil, high oleic safflower oil, medium chain triglyceride oil (MCT oil), high gamma linolenic (GLA) safflower oil, sunflower oil, high oleic sunflower oil, palm oil, palm kernel oil, palm olein, canola oil, high oleic canola oil, marine oils, fish oils, algal oils, borage oil, cottonseed oil, fungal oils, eicosapentaenoic acid (EPA), docosahexaenoic acid (DHA), arachidonic acid (ARA), conjugated linoleic acid (CLA), α-linolenic acid, rice bran oil, wheat bran oil, interesterified oils, transesterified oils, structured lipids, and combinations thereof. Generally, the fats used in a nutritional composition for formulating infant formulas and pediatric formulas provide fatty acids needed both as an energy source and for the healthy development of the infant, toddler, or child. These fats typically comprise triglycerides, although the fats may also comprise diglycerides, monoglycerides, and free fatty acids. Fatty acids provided by the fats in the nutritional composition include, but are not limited to, capric acid, lauric acid, myristic acid, palmitic acid, palmitoleic acid, stearic acid, oleic acid, linoleic acid, α-linolenic acid, ARA, EPA, and DHA. The nutritional composition can include any individual source of fat or combination of the various sources of fat grouped above. Preferably, the fat is a mixture of vegetable fat and milk fat such as obtained from milk from a mammal like cow, sheep, goat, mare, or camel. More preferably, wherein the milk fat is bovine milk fat. Mixtures of different types of fat are preferred because they help to provide different fatty acids and better resemble the type of linkage between the glycerol moiety and the fatty acid moiety in the fat, when compared to human mother's milk.

When one or more prebiotics is/are present, then it is preferred that the weight ratio between the oligosaccharide according to the disclosure and the prebiotic(s) is in the range of from 0.5:10 to 10:0.5.

Preferably, the pharmaceutical or nutritional composition according to the disclosure comprises one or more probiotics for its beneficial effect on the subject's gut microbiome. Examples of probiotics include Bifidobacterium, Lactobacillus and Saccharomyces boulardii.

In a more preferred embodiment, the nutritional composition is a synthetic nutritional composition.

In an additional and/or alternative more preferred embodiment, the nutritional composition is selected from a group consisting of a milk replacer, creep feed, prestarter diet, starter diet, pre-weaning feed, weaning feed and post-weaning feed, preferably selected form the group consisting of a milk replacer, starter diet, creep feed and post-weaning feed, more preferably the nutritional composition is a milk replacer or starter diet, most preferably a milk replacer.

In the context of the disclosure, the term “creep feed” refers to a nutritional composition, which is used to supplement a pre-weaned animal with a solid diet while the animal is suckling. For example, a piglet receives creep feed to ease the transition from sow's milk to solid pig starter feed. The creep feed stimulates the digestive system of the piglet to produce, e.g., amylase, which digests carbohydrates in dry feed. If a pig is better able to digest dry feed, he can start eating quickly post-weaning for better performance.

In the context of the disclosure, the term “milk replacer” refers to a nutritional composition that serves as a substitute for mother's milk. A milk replacer comprises milk proteins, fat, carbohydrates, vitamins and minerals. Preferably, a milk replacer comprises 10 drywt. % to 28 drywt. % of at least one protein, 13 drywt. % to 28 drywt. % (preferably 20 drywt. % to 25 drywt. %) of at least one fat and lactose at <55 drywt. %. More preferably, a milk replacer comprises 18 drywt. % to 24 drywt. % of at least one protein, 15 drywt. % to 28 drywt. % (preferably 20 drywt. % to 25 drywt. %) of at least one fat and lactose at <50 drywt. %. In this context, “drywt. %” refers to the ratio between the weight of the recited component (e.g., 2′FL) and the dry weight of the milk replacer (w/v %). Throughout the disclosure and claims, a milk replacer is preferably a calf milk replacer.

Method for Preventing and/or Treating Shipping Fever

In a second aspect, the disclosure provides a method for preventing and/or treating shipping fever in an animal, preferably wherein the animal is a farm animal or a domesticated animal, more preferably a farm animal. The method comprises administering a fucosylated mammalian milk oligosaccharide (MMO), preferably a composition comprising a fucosylated mammalian milk oligosaccharide (MMO). The fucosylated MMO and the composition are as described in the first aspect of the disclosure.

In the context of the disclosure, the “method for preventing and/or treating”, “shipping fever”, “animal”, “composition”, and “fucosylated mammalian milk oligosaccharide” as described throughout the second aspect of the disclosure and claims are as described in the first aspect of the disclosure.

### Use

In a third aspect, the disclosure provides the use of a fucosylated mammalian milk oligosaccharide (MMO), preferably the use of a composition comprising a fucosylated mammalian milk oligosaccharide (MMO) (composition as described in the first aspect of the disclosure), for the manufacture of a medicament for preventing and/or treating shipping fever in an animal., preferably wherein the animal is a farm animal or a domesticated animal., more preferably a farm animal.

In the context of the disclosure, the “method for preventing and/or treating”, “shipping fever”, “animal”, “composition”, and “fucosylated mammalian milk oligosaccharide” as described throughout the third aspect of the disclosure and claims are as described in the first aspect of the disclosure.

Method for Preventing and/or Treating Mycoplasma bovis

In a fourth aspect, the disclosure provides a method for preventing and/or treating Mycoplasma bovis in an animal, preferably wherein the animal is a farm animal or a domesticated animal, more preferably a farm animal. The method comprises administering a fucosylated mammalian milk oligosaccharide (MMO), preferably a composition comprising a fucosylated mammalian milk oligosaccharide (MMO). The fucosylated MMO and the composition are as described in the first aspect of the disclosure.

In the context of the disclosure, the “method for preventing and/or treating”, “animal”, “composition”, and “fucosylated mammalian milk oligosaccharide” as described throughout the fourth aspect of the disclosure and claims are as described in the first aspect of the disclosure.

Method for Preventing and/or Treating Adenovirus Type 3

In a fifth aspect, the disclosure provides a method for preventing and/or treating adenovirus type 3 in an animal, preferably wherein the animal is a farm animal or a domesticated animal, more preferably a farm animal. The method comprises administering a fucosylated mammalian milk oligosaccharide (MMO), preferably a composition comprising a fucosylated mammalian milk oligosaccharide (MMO). The fucosylated MMO and the composition are as described in the first aspect of the disclosure.

In the context of the disclosure, the “method for preventing and/or treating”, “animal”, “composition”, and “fucosylated mammalian milk oligosaccharide” as described throughout the fifth aspect of the disclosure and claims are as described in the first aspect of the disclosure.

### Specific Embodiments

This disclosure preferably relates to the following specific embodiments:
1. A fucosylated mammalian milk oligosaccharide (MMO) for use in a method for preventing and/or treating shipping fever in an animal., preferably in a farm or domesticated animal., more preferably in a farm animal.
2. A fucosylated MMO for use according to embodiment 1, wherein the method is for preventing and/or treating shipping fever pneumonia and/or shipping fever diarrhea, preferably for preventing and/or treating shipping fever pneumonia.
3. A fucosylated MMO for use according to embodiment 2, wherein the pneumonia is acute pneumonia.
4. A fucosylated MMO for use according to any one of embodiments 1 to 3, wherein the animal, preferably the farm animal or domesticated animal, is selected from the group consisting of bovine, pig, sheep, goat, horse, camelid (preferably llama or alpaca), dog, cat, rabbit and poultry; more preferably selected from the group consisting of bovine, pig, sheep, goat, horse, camelid (preferably llama or alpaca) and poultry; even more preferably selected from the group consisting of bovine, sheep, goat, horse, camelid (preferably llama or alpaca) and poultry; even more preferably selected from the group consisting of bovine, horse and poultry; even more preferably the animal is bovine or a horse; most preferably the animal is bovine.
5. A fucosylated MMO for use according to any one of embodiments 1 to 4, wherein the animal is a non-adult animal.
6. A fucosylated MMO for use according to embodiment 5, wherein the animal, preferably the farm animal or domesticated animal, is selected from the group consisting of a calf, piglet, lamb, kid, foal, cria, puppy, kitten, kit and a chick; more preferably selected from the group consisting of calf, piglet, lamb, kid, foal, cria and chick; even more preferably selected from the group consisting of a calf, lamb, kid, foal, cria and a chick; even more preferably selected from the group consisting of a calf, foal and chick; even more preferably the animal is a calf or a foal; most preferably the animal is a calf.
7. A fucosylated MMO for use according to any one of embodiments 1 to 6, wherein the fucosylated MMO consists of 3-8, preferably 3-7, more preferably 3-6, even more preferably 3-5, monosaccharides.
8. A fucosylated MMO for use according to any one of embodiments 1 to 7, wherein the fucosylated MMO is a neutral oligosaccharide.
9. A fucosylated MMO for use according to any one of embodiments 1 to 8, wherein the fucosylated MMO comprises a fucose that is linked to a monosaccharide in an α-1,2-, α-1,3- or α-1,4-linkage, preferably an α-1,2- or an α-1,3-linkage, more preferably an α-1,2-linkage, and wherein the monosaccharide is preferably selected from glucose, N-acetylglucosamine and galactose, more preferably the monosaccharide is glucose or galactose, even more preferably the monosaccharide is galactose.
10. A fucosylated MMO for use according to any one of embodiments 1 to 9, wherein the fucosylated MMO comprises a lactose, a lacto-N-biose (LNB) or N-acetyllactosamine (LacNAc) at its reducing end, preferably the fucosylated oligosaccharide comprises lactose or LacNAc at is reducing end, more preferably the fucosyalted oligosaccharide comprises lactose at its reducing end.
11. A fucosylated MMO for use according to any one of embodiments 1 to 10, wherein the fucosylated MMO is selected from the group consisting of 2′-fucosyllactose (2′FL), 3-fucosyllactose (3-FL), difucosyllactose (diFL), 2′-fucosyl-N-acetyllactosamine (2′FlacNAc), difucosyl-N-acetyllactosamine (diFLacNAc), 3-fucosyl-N-acetyllactosamine (3FlacNAc), 2′-fucosyllacto-N-biose (2′FLNB), 4-fucosyllacto-N-biose (4FLNB), difucosyllacto-N-biose (diFLNB), lacto-N-fucopentaose I (LNFP I), blood group A antigen hexaose type 1 (GalNAc-LNFP I), blood group B antigen hexaose type 1 (Gal-LNFP I), lacto-N-fucopentaose II (LNFP II), lacto-N-fucopentaose III (LNFP III), lacto-N-fucopentaose V (LNFP V), lacto-N-difucohexaose I (LNDFH I), lacto-N-difucohexaose II (LNDFH II), lewis b-lewis x, monofucosyllacto-N-hexaose III (MFLNH III), difucosyllacto-N-hexaose (a) (DFLNH (a)), difucosyllacto-N-hexaose (DFLNH), trifucosyllacto-N-hexaose (TFLNH), lacto-N-neofucopentaose I (LNnFP I), lacto-N-neofucopentaose V (LNnFP V, LNFP VI), and lacto-N-neodifucohexaose (LNnDFH); more preferably selected from the group consisting of 2′FL, diFL, LNFP I, GalNAc-LNFP I, Gal-LNFP I, LNDFH I, lewis b-lewis x, DFLNH (a), TFLNH, LNnFP I, 2′FLNB, diFLNB, 2′FlacNAc and diFLacNAc; even more preferably selected from the group consisting of 2′FL, diFL, LNFP I, LNnFP I, 2′FLNB, diFLNB, 2′FlacNAc and diFLacNAc; even more preferably selected from the group consisting of 2′FL, diFL, 2′FLNB, diFLNB, 2′FlacNAc and diFLacNAc; even more preferably selected from the group consisting of 2′FL, 2′FLNB and 2′FlacNAc; even more preferably wherein the fucosylated MMO is 2′FL or diFL, most preferably wherein the fucosylated MMO is 2′FL.
12. A fucosylated MMO for use according to any one of embodiments 1 to 11, wherein the fucosylated MMO is selected from the group consisting of 2′FL, diFL, LNFP I, LNnFP I, 2′FLNB, diFLNB, 2′FlacNAc and diFLacNAc.
13. A fucosylated MMO for use according to any one of embodiments 1 to 12, wherein the fucosylated MMO is 2′-fucosyllactose (2′FL).
14. A fucosylated MMO for use according to any one of embodiments 1 to 13, wherein the fucosylated MMO is part of a composition.
15. A fucosylated MMO for use according to embodiment 14, wherein the fucosylated MMO is present at 0.001-1.000 wt. %, preferably 0.001-0.750 wt. %, more preferably 0.001-0.500 wt. %, even more preferably 0.001-0.400 wt. %, even more preferably 0.005-0.400 wt. %, even more preferably 0.005-0.300 wt. %, even more preferably 0.005-0.250 wt. %, most preferably 0.005-0.100 wt. %, of the total weight of the composition.
16. A fucosylated MMO for use according to embodiment 14 or 15, wherein the fucosylated MMO is present at 0.001-0.250 wt. %, preferably 0.001-0.200 wt. %, more preferably 0.001-0.100 wt. %, even more preferably 0.001-0.020 wt. %, even more preferably 0.001-0.010 wt. %, even more preferably 0.002-0.010 wt. %, even more preferably 0.003-0.010 wt. %, even more preferably 0.003-0.0075 wt. %, most preferably 0.003-0.006 wt. %, of the total weight of the composition; preferably wherein the animal is a bovine or a horse, more preferably a calf or a foal., even more preferably a calf.
17. A fucosylated MMO for use according to embodiment 14 or 15, wherein the fucosylated MMO is present at 0.010-1.000 wt. %, preferably 0.010-0.750 wt. %, more preferably 0.010-0.500 wt. %, even more preferably 0.010-0.400 wt. %, even more preferably 0.050-0.400 wt. %, even more preferably 0.100-0.400 wt. %, most preferably 0.100-0.300 wt. %, of the total weight of the composition; preferably wherein the animal is a pig, a sheep, a goat or a camelid (preferably llama or alpaca), more preferably wherein the animal is a piglet, a lamb, a kid or a cria, even more preferably wherein the animal is a piglet, a lamb or a kid, most preferably wherein the animal is a piglet.
18. A fucosylated MMO for use according to any one of embodiments 1 to 17, wherein the method comprises administering an effective amount of the fucosylated MMO.
19. A fucosylated MMO for use according to any one of embodiments 1 to 18, wherein the fucosylated MMO is administered to the animal at least once, preferably at least twice, between birth (0 weeks) and 5 weeks of age, preferably between birth and 4 weeks of age, more preferably between birth and 3 weeks of age.
20. A fucosylated MMO for use according to any one of embodiments 1 to 19, wherein the fucosylated MMO is administered to the animal at least once, preferably at least twice, more preferably daily:
   in the three weeks, preferably two weeks, more preferably one week, preceding the day of transporting the animal; and/or
   on the day of transporting the animal; and/or
   in the three weeks, preferably two weeks, more preferably one week, after the day of transporting the animal.
21. A fucosylated MMO for use according to any one of embodiments 1 to 20, wherein the fucosylated MMO is administered to the animal for at least 1 week, preferably at least 2 weeks, more preferably at least 3 weeks, even more preferably at least 4 weeks, most preferably at least 6 weeks.
22. A fucosylated MMO for use according to any one of embodiments 1 to 21, wherein the fucosylated MMO is administered to the animal for ≤32 weeks, preferably ≤28 weeks, more preferably ≤24 weeks, even more preferably ≤20 weeks, even more preferably ≤16 weeks, even more preferably ≤12 weeks, most preferably ≤6 weeks.
23. A fucosylated MMO for use according to any one of embodiments 1 to 22, wherein the fucosylated MMO is administered to the animal for 1-36, preferably 1-30, even more preferably 1-28, even more preferably 1-24, even more preferably 1-18, even more preferably 1-12, even more preferably 1-8, even more preferably 1-6, even more preferably 1-4, most preferably 2-4, consecutive weeks.
24. A fucosylated MMO for use according to any one of embodiments 1 to 23, wherein the fucosylated MMO is administered to the animal for:
   if subject is bovine: 1-36, preferably 1-32, more preferably 1-30, even more preferably 1-28, even more preferably 1-24, even more preferably 1-18, even more preferably 1-12, even more preferably 2-12, most preferably 3-12, consecutive weeks;
   if subject is a pig: 1-18, preferably 1-12, more preferably 1-8, even more preferably 1-6, even more preferably 1-4, most preferably 2-4, consecutive weeks;
   if subject is a sheep: 1-36, preferably 1-32, more preferably 1-30, even more preferably 1-28, even more preferably 1-24, even more preferably 1-18, even more preferably 1-12, even more preferably 1-8, even more preferably 1-6, even more preferably 1-4, most preferably 2-4, consecutive weeks;
   if subject is a goat: 1-36, preferably 1-32, more preferably 1-30, even more preferably 1-28, even more preferably 1-24, even more preferably 1-18, even more preferably 1-12, even more preferably 1-8, even more preferably 1-6, even more preferably 1-4, most preferably 2-4, consecutive weeks;
   if subject is a horse: 1-18, preferably 1-12, more preferably 1-8, even more preferably 1-6, even more preferably 1-4, most preferably 2-4, consecutive weeks;
   if subject is a camelid: 1-18, preferably 1-12, more preferably 1-8, even more preferably 1-6, even more preferably 1-4, most preferably 2-4, consecutive weeks;
   if subject is a rabbit: 1-12, preferably 1-8, more preferably 1-6, even more preferably 1-4, most preferably 2-4, consecutive weeks;
   if subject is a dog: 1-12, preferably 1-8, more preferably 1-6, even more preferably 1-4, most preferably 2-4, consecutive weeks;
   if subject is a cat: 1-12, preferably 1-8, more preferably 1-6, even more preferably 1-4, most preferably 2-4, consecutive weeks;
   if subject is poultry: 1-8, preferably 1-6, more preferably 1-4, most preferably 2-4, consecutive weeks.
25. A fucosylated MMO for use according to any one of embodiments 1 to 24, wherein the fucosylated MMO is administered at least once a week, preferably at least once every 3 days, more preferably at least once every 2 days, most preferably at least once daily.
26. A fucosylated MMO for use according to any one of embodiments 1 to 25, wherein the fucosylated MMO is administered at a daily dose of 0.0001-15.0 g, preferably 0.0001-10.0 g, more preferably 0.0001-5.0 g, even more preferably 0.0001-2.0 g, even more preferably 0.0001-1.0 g, even more preferably 0.0005-1.0 g, most preferably 0.0010-1.0 g.
27. A fucosylated MMO for use according to any one of embodiments 1 to 25, wherein the fucosylated MMO is administered at a daily dose of 0.01-15.0 g, preferably 0.01-10.0 g, more preferably 0.01-5.0 g, even more preferably 0.01-2.0 g, even more preferably 0.01-1.0 g, even more preferably 0.015-1.0 g, most preferably 0.02-1.0 g.
28. A fucosylated MMO for use according to any one of embodiments 1 to 25, wherein the fucosylated MMO is administered at a daily dose of 0.01-1.0 g, preferably 0.01-0.75 g, more preferably 0.01-0.65 g, even more preferably 0.01-0.60 g, even more preferably 0.01-0.50 g, even more preferably 0.015-0.50 g, even more preferably 0.015-0.40 g, even more preferably 0.015-0.30 g, even more preferably 0.015-0.20 g, most preferably 0.015-0.10 g; preferably wherein the animal is a bovine or a horse, more preferably a calf or a foal, even more preferably a calf.
29. A fucosylated MMO for use according to any one of embodiments 1 to 25, wherein the fucosylated MMO is administered at a daily dose of 0.01-5.0 g, preferably 0.1-2.5 g, more preferably 0.1-1.5 g, even more preferably 0.1-1.2 g, even more preferably 0.2-1.2 g, even more preferably 0.3-1.2 g, even more preferably 0.4-1.2 g, most preferably 0.5-1.2 g; preferably wherein the animal is a pig, a sheep, a goat or a camelid (preferably llama or alpaca), more preferably wherein the animal is a piglet, a lamb, a kid or a cria, even more preferably wherein the animal is a piglet, a lamb or a kid, most preferably wherein the animal is a piglet.
30. A fucosylated MMO for use according to any one of embodiments 1 to 29, wherein the fucosylated MMO is administered at a daily dose of 0.01-150.0 mg, preferably 0.01-125.0 mg, more preferably 0.01-100.0 mg, even more preferably 0.05-100.0 mg, even more preferably 0.1-100.0 mg, most preferably 0.1-80.0 mg, per kg bodyweight of the animal.
31. A fucosylated MMO for use according to any one of embodiments 1 to 29, wherein the fucosylated MMO is administered at a daily dose of 0.1-50.0 mg, preferably 0.1-30.0 mg, more preferably 0.1-25.0 mg, even more preferably 0.1-20.0 mg, even more preferably 0.1-15.0 mg, even more preferably 0.1-10.0 mg, even more preferably 0.25-10.0 mg, most preferably 0.5-10.0 mg, per kg bodyweight of the animal; preferably wherein the animal is a bovine or a horse, more preferably a calf or a foal, even more preferably a calf.
32. A fucosylated MMO for use according to any one of embodiments 1 to 29, wherein the fucosylated MMO is administered at a daily dose of 0.1-150.0 mg, preferably 0.1-125.0 mg, more preferably 1.0-125.0 mg, even more preferably 5.0-125.0 mg, even more preferably 5.0-100.0 mg, even more preferably 5.0-80.0 mg, most preferably 10.0-80.0 mg, per kg bodyweight of the animal; preferably wherein the animal is a pig, a sheep, a goat or a camelid (preferably llama or alpaca), more preferably wherein the animal is a piglet, a lamb, a kid or a cria, even more preferably wherein the animal is a piglet, a lamb or a kid, most preferably wherein the animal is a piglet.
33. A fucosylated MMO for use according to any one of embodiments 1 to 32, wherein the method further comprises administering a second mammalian milk oligosaccharide (MMO), preferably wherein the second mammalian milk oligosaccharide is a sialylated MMO, more preferably wherein the second mammalian milk oligosaccharide is a sialylated MMO comprising a lactose, a lacto-N-biose (LNB) or N-acetyllactosamine (LacNAc) at its reducing end, even more preferably the sialylated MMO comprises lactose or LacNAc at is reducing end, most preferably the sialylated MMO comprises lactose at its reducing end.
34. A fucosylated MMO for use according to embodiment 33, wherein the second MMO is selected from the group consisting of 3′-sialyllactose (3′SL), 6′-sialyllactose (6′SL), 3,6-disialyllactose, 6,6′-disialyllactose, 8,3-disialyllactose, 3′S-2′FL, 6′S-2′FL, 3′S-3-FL, 6′S-3-FL, LST a, LST b, LST c, LST d, 3′-sialyllacto-N-biose (3′SLNB), 6′-sialyllacto-N-biose (6′SLNB), sialyl Lewis a, 3′-sialyllactosamine (3′SLacNAc), 6′-sialyllactosamine (6′SLacNAc) and sialyl Lewis x; preferably the second MMO is selected from the group consisting of 3′SL, 6′SL, LST a, LST b, LST c, LST d, 3′SLNB, 6′SLNB, sialyl Lewis a, 3′SLacNAc, 6′SLacNAc and sialyl Lewis x; more preferably the second MMO is selected from the group consisting of 3′SL, 6′SL, 3′SLNB, 6′SLNB, 3′SLacNAc and 6′SLacNAc; most preferably the second MMO is 3′-sialyllactose (3′SL) or 6′-sialyllactose (6′SL).
35. A fucosylated MMO for use according to embodiment 33 or 34, wherein the second MMO is 3′-sialyllactose (3′SL) or 6′-sialyllactose (6′SL).
36. A fucosylated MMO for use according to any one of embodiments 33 to 35, wherein the fucosylated MMO and the second MMO are part of a composition.
37. A fucosylated MMO for use according to any one of embodiments 33 to 36, wherein the amount of the fucosylated MMO administered to the animal is lower than the amount of the second MMO administered to the animal, preferably the amount of the fucosylated MMO is 1.1 to 15.0, more preferably 1.5 to 15.0, even more preferably 2.5 to 15.0, even more preferably 5.0 to 15.0, even more preferably 5.0 to 12.5, most preferably 5.0 to 10.0, times lower than the amount of the second MMO administered to the animal.
38. A fucosylated MMO for use according to any one of embodiments 33 to 36, wherein the amount of the fucosylated MMO administered to the animal is lower than the amount of the second MMO administered to the animal, preferably the amount of the fucosylated MMO is 1.1 to 10.0, preferably 1.5-7.5, more preferably 2.5-7.5, most preferably 4.0-7.0, times lower than the amount of the second MMO administered to the animal.
39. A fucosylated MMO for use according to any one of embodiments 33 to 36, wherein the amount of the second MMO administered to the animal is 0.1-30.0, preferably 0.1-25.0, more preferably 0.1-20.0, even more preferably 0.1-15.0, even more preferably 0.2-15.0, even more preferably 0.3-15.0, even more preferably 0.4-15.0, most preferably 0.5-15.0, times the amount of the fucosylated MMO administered to the animal.
40. A fucosylated MMO for use according to any one of embodiments 1 to 39, wherein the method further comprises administering a third mammalian milk oligosaccharide (MMO), preferably wherein the third mammalian milk oligosaccharide is a sialylated MMO, more preferably wherein the third mammalian milk oligosaccharide is a sialylated MMO comprising a lactose, a lacto-N-biose (LNB) or N-acetyllactosamine (LacNAc) at its reducing end, even more preferably the sialylated MMO comprises lactose or LacNAc at is reducing end, most preferably the sialylated MMO comprises lactose at its reducing end.
41. A fucosylated MMO for use according to embodiment 40, wherein the third MMO is selected from the group consisting of 3′-sialyllactose (3′SL), 6′-sialyllactose (6′SL), 3,6-disialyllactose, 6,6′-disialyllactose, 8,3-disialyllactose, 3′S-2′FL, 6′S-2′FL, 3′S-3-FL, 6′S-3-FL, LST a, LST b, LST c, LST d, 3′-sialyllacto-N-biose (3′SLNB), 6′-sialyllacto-N-biose (6′SLNB), sialyl Lewis a, 3′-sialyllactosamine (3′SLacNAc), 6′-sialyllactosamine (6′SLacNAc) and sialyl Lewis x; preferably the third MMO is selected from the group consisting of 3′SL, 6′SL, LST a, LST b, LST c, LST d, 3′SLNB, 6′SLNB, sialyl Lewis a, 3′SLacNAc, 6′SLacNAc and sialyl Lewis x; more preferably the third MMO is selected from the group consisting of 3′SL, 6′SL, 3′SLNB, 6′SLNB, 3′SLacNAc and 6′SLacNAc; most preferably the third MMO is 6′-sialyllactose (6′SL) or 3′-sialyllactose (3′SL).
42. A fucosylated MMO for use according to embodiment 40 or 41, wherein the third MMO is 6′-sialyllactose (6′SL) or 3′-sialyllactose (3′SL).
43. A fucosylated MMO for use according to any one of embodiments 40 to 42, wherein the fucosylated MMO, the second MMO and the third MMO are part of a composition.
44. A fucosylated MMO for use according to any one of embodiments 40 to 43, wherein the amount of the fucosylated MMO administered to the animal is lower than the amount of the third MMO administered to the animal, preferably the amount of the fucosylated MMO is 1.1 to 15.0, more preferably 1.5 to 15.0, even more preferably 2.5 to 15.0, even more preferably 5.0 to 15.0, even more preferably 5.0 to 12.5, most preferably 5.0 to 10.0, times lower than the amount of the third MMO administered to the animal.
45. A fucosylated MMO for use according to any one of embodiments 40 to 43, wherein the amount of the fucosylated MMO administered to the animal is lower than the amount of the third MMO administered to the animal, preferably the amount of the fucosylated MMO is 1.1 to 10.0, preferably 1.5-7.5, more preferably 1.5-5.0, most preferably 2.5-5.0, times lower than the amount of the third MMO administered to the animal.
46. A fucosylated MMO for use according to any one of embodiments 40 to 43, wherein the amount of the third MMO administered to the animal is 0.1-30.0, preferably 0.1-25.0, more preferably 0.1-20.0, even more preferably 0.1-15.0, even more preferably 0.2-15.0, even more preferably 0.3-15.0, even more preferably 0.4-15.0, most preferably 0.5-15.0, times the amount of the fucosylated MMO administered to the animal.
47. A fucosylated MMO for use according to any one of embodiments 40 to 46, wherein the ratio between the amount of the second MMO and the amount of the third MMO administered to the animal is similar to this ratio as found in the mother's milk of the same species.
48. A fucosylated MMO for use according to any one of embodiments 40 to 47, wherein the amount of the second MMO administered to the animal is higher than the amount of the third MMO administered to the animal, preferably the amount of the second MMO is 1.1 to 40.0, more preferably 1.1 to 35.0, most preferably 1.5 to 35.0, times the amount of the third MMO administered to the animal.
49. A fucosylated MMO for use according to any one of embodiments 40 to 47, wherein the amount of the second MMO administered to the animal is higher than the amount of the third MMO administered to the animal, preferably the amount of the second MMO is 1.1 to 6.0, more preferably 1.1 to 5.0, even more preferably 1.25 to 4.0, even more preferably 1.25-3.0, even more preferably 1.5 to 3.0, most preferably 1.5-2.5, times higher than the amount of the third MMO administered to the animal.
50. A fucosylated MMO for use according to any one of embodiments 1 to 49, wherein the method does not comprise administering lacto-N-tetraose (LNT).
51. A fucosylated MMO for use according to any one of embodiments 1 to 50, wherein the method does not comprise administering lacto-N-neotetraose (LNnT).
52. A fucosylated MMO for use according to any one of embodiments 1 to 51, wherein the method does not comprise administering N-acetyllactosamine.
53. A fucosylated MMO for use according to any one of embodiments 1 to 52, wherein the method does not comprise administering an oligosaccharide containing N-acetyllactosamine, preferably does not comprise administering a saccharide containing N-acetyllactosamine.
54. A fucosylated MMO for use according to any one of embodiments 1 to 53, wherein the method does not comprise administering a fructo-oligosaccharide (FOS).
55. A fucosylated MMO for use according to any one of embodiments 1 to 54, wherein the method does not comprise administering a galacto-oligosaccharide (GOS).
56. A fucosylated MMO for use according to any one of embodiments 1 to 55, wherein the method does not comprise administering an isomaltooligosaccharide.
57. A fucosylated MMO for use according to any one of embodiments 1 to 56, wherein the method does not comprise administering a neutral non-fucosylated MMO.
58. A fucosylated MMO for use according to any one of embodiments 1 to 32 and 50 to 57, wherein the method does not comprise administering a further mammalian milk oligosaccharide.
59. A fucosylated MMO for use according to any one of embodiments 1 to 58, wherein the method further comprises administering a microbial strain that does not use, preferably is not capable to use, the fucosylated MMO as carbon source.
60. A fucosylated MMO for use according to any one of embodiments 1 to 58, wherein the method does not comprise administering a microbial strain.
61. A fucosylated MMO composition for use according to any one of embodiments 14 to 17, 36 and 43, wherein the composition is not mother's milk.
62. A fucosylated MMO for use according to any one of embodiments 14 to 17, 36, 43 and 61, wherein the composition is a synthetic composition.
63. A fucosylated MMO for use according to any one of embodiments 1 to 62, wherein the fucosylated MMO has been produced in vitro and/or ex vivo by a cell, preferably a single cell, wherein the cell is preferably chosen from the group consisting of a microorganism, a plant cell, an animal cell and a protozoan cell.
64. A fucosylated MMO for use according to any one of embodiments 1 to 63, wherein the fucosylated MMO is linked, preferably chemically linked, to a carrier for delivery of the fucosylated MMO.
65. A fucosylated MMO for use according to embodiment 64, wherein the linker is a ceramide-based carrier or a polypeptide-based carrier, preferably a ceramide-based carrier.
66. A fucosylated MMO for use according to embodiment 65, wherein the ceramide-based carrier is selected from a group consisting of d18:1/16:0, t18:0-16:0, t18:0-h16:0, t18:0-h22:0 and t18:0-h24:0.
67. A fucosylated MMO for use according to any one of embodiments 1 to 66, wherein the fucosylated MMO is encapsulated.
68. A method for preventing and/or treating shipping fever in an animal., preferably a farm animal or a domesticated animal, more preferably a farm animal, wherein the method comprises administering a fucosylated MMO as defined in any one of embodiments 1 to 67.
69. A method according to embodiment 68, wherein the method is for preventing and/or treating shipping fever pneumonia and/or shipping fever diarrhea, preferably for preventing and/or treating shipping fever pneumonia.
70. A method according to embodiment 69, wherein the pneumonia is acute pneumonia.
71. A method according to any one of embodiments 68 to 70, wherein the animal is as defined in any one of embodiments 4 to 6.
72. A method according to any one of embodiments 68 to 71, wherein the method comprises administering an effective amount of the fucosylated MMO to the animal., preferably wherein the fucosylated MMO is administered as defined in any one of embodiments 19 to 32.
73. Use of a fucosylated MMO as defined in any one of embodiments 1 to 67 for the manufacture of a medicament for preventing and/or treating shipping fever in an animal, preferably wherein the animal is a farm animal or a domesticated animal, more preferably wherein the animal is a farm animal.
74. Use according to embodiment 73, wherein the medicament is for preventing and/or treating shipping fever pneumonia and/or shipping fever diarrhea, preferably for preventing and/or treating shipping fever pneumonia.
75. Use according to embodiment 74, wherein the pneumonia is acute pneumonia.
76. Use according to any one of embodiments 73 to 75, wherein the animal is as defined in any one of embodiments 4 to 6.
77. Use according to any one of embodiments 73 to 76, wherein the method comprises administering an effective amount of the fucosylated MMO to the animal, preferably wherein the fucosylated MMO is administered as defined in any one of embodiments 19 to 32.

In addition, the disclosure preferably relates to the following specific embodiments:
1. A composition comprising a fucosylated mammalian milk oligosaccharide (MMO), wherein the composition is for use in a method for preventing and/or treating shipping fever in an animal., preferably in a farm or domesticated animal, more preferably in a farm animal.
2. A composition for use according to embodiment 1, wherein the method is for preventing and/or treating shipping fever pneumonia and/or shipping fever diarrhea, preferably for preventing and/or treating shipping fever pneumonia.
3. A composition for use according to embodiment 2, wherein the pneumonia is acute pneumonia.
4. A composition for use according to any one of embodiments 1 to 3, wherein the animal., preferably the farm animal or domesticated animal, is selected from the group consisting of bovine, pig, sheep, goat, horse, camelid (preferably llama or alpaca), dog, cat, rabbit and poultry; more preferably selected from the group consisting of bovine, pig, sheep, goat, horse, camelid (preferably llama or alpaca) and poultry; even more preferably selected from the group consisting of bovine, sheep, goat, horse, camelid (preferably llama or alpaca) and poultry; even more preferably selected from the group consisting of bovine, horse and poultry; even more preferably the animal is bovine or a horse; most preferably the animal is bovine.
5. A composition for use according to any one of embodiments 1 to 4, wherein the animal is a non-adult animal.
6. A composition for use according to embodiment 5, wherein the animal, preferably the farm animal or domesticated animal, is selected from the group consisting of a calf, piglet, lamb, kid, foal, cria, puppy, kitten, kit and a chick; more preferably selected from the group consisting of calf, piglet, lamb, kid, foal, cria and chick; even more preferably selected from the group consisting of a calf, lamb, kid, foal, cria and a chick; even more preferably selected from the group consisting of a calf, foal and chick; even more preferably the animal is a calf or a foal; most preferably the animal is a calf.
7. A composition for use according to any one of embodiments 1 to 6, wherein the fucosylated MMO consists of 3-8, preferably 3-7, more preferably 3-6, even more preferably 3-5, monosaccharides.
00. A composition for use according to any one of embodiments 1 to 7, wherein the fucosylated MMO is a neutral oligosaccharide.
9. A composition for use according to any one of embodiments 1 to 8, wherein the fucosylated MMO comprises a fucose that is linked to a monosaccharide in an α-1,2-, α-1,3- or α-1,4-linkage, preferably an α-1,2- or an α-1,3-linkage, more preferably an α-1,2-linkage, and wherein the monosaccharide is preferably selected from glucose, N-acetylglucosamine and galactose, more preferably the monosaccharide is glucose or galactose, even more preferably the monosaccharide is galactose.
10. A composition for use according to any one of embodiments 1 to 9, wherein the fucosylated MMO comprises a lactose, a lacto-N-biose (LNB) or N-acetyllactosamine (LacNAc) at its reducing end, preferably the fucosylated oligosaccharide comprises lactose or LacNAc at is reducing end, more preferably the fucosyalted oligosaccharide comprises lactose at its reducing end.
11. A composition for use according to any one of embodiments 1 to 10, wherein the fucosylated MMO is selected from the group consisting of 2′-fucosyllactose (2′FL), 3-fucosyllactose (3-FL), difucosyllactose (diFL), 2′-fucosyl-N-acetyllactosamine (2′FlacNAc), difucosyl-N-acetyllactosamine (diFLacNAc), 3-fucosyl-N-acetyllactosamine (3FlacNAc), 2′-fucosyllacto-N-biose (2′FLNB), 4-fucosyllacto-N-biose (4FLNB), difucosyllacto-N-biose (diFLNB), lacto-N-fucopentaose I (LNFP I), blood group A antigen hexaose type 1 (GalNAc-LNFP I), blood group B antigen hexaose type 1 (Gal-LNFP I), lacto-N-fucopentaose II (LNFP II), lacto-N-fucopentaose III (LNFP III), lacto-N-fucopentaose V (LNFP V), lacto-N-difucohexaose I (LNDFH I), lacto-N-difucohexaose II (LNDFH II), lewis b-lewis x, monofucosyllacto-N-hexaose III (MFLNH III), difucosyllacto-N-hexaose (a) (DFLNH (a)), difucosyllacto-N-hexaose (DFLNH), trifucosyllacto-N-hexaose (TFLNH), lacto-N-neofucopentaose I (LNnFP I), lacto-N-neofucopentaose V (LNnFP V, LNFP VI), and lacto-N-neodifucohexaose (LNnDFH); more preferably selected from the group consisting of 2′FL, diFL, LNFP I, GalNAc-LNFP I, Gal-LNFP I, LNDFH I, lewis b-lewis x, DFLNH (a), TFLNH, LNnFP I, 2′FLNB, diFLNB, 2′FlacNAc and diFLacNAc; even more preferably selected from the group consisting of 2′FL, diFL, LNFP I, LNnFP I, 2′FLNB, diFLNB, 2′FlacNAc and diFLacNAc; even more preferably selected from the group consisting of 2′FL, diFL, 2′FLNB, diFLNB, 2′FlacNAc and diFLacNAc; even more preferably selected from the group consisting of 2′FL, 2′FLNB and 2′FlacNAc; even more preferably wherein the fucosylated MMO is 2′FL or diFL, most preferably wherein the fucosylated MMO is 2′FL.
12. A composition for use according to any one of embodiments 1 to 11, wherein the fucosylated MMO is selected from the group consisting of 2′FL, diFL, LNFP I, LNnFP I, 2′FLNB, diFLNB, 2′FlacNAc and diFLacNAc.
13. A composition for use according to any one of embodiments 1 to 12, wherein the fucosylated MMO is 2′-fucosyllactose (2′FL).
14. A composition for use according to any one of embodiments 1 to 13, wherein the fucosylated MMO is present at 0.001-1.000 wt. %, preferably 0.001-0.750 wt. %, more preferably 0.001-0.500 wt. %, even more preferably 0.001-0.400 wt. %, even more preferably 0.005-0.400 wt. %, even more preferably 0.005-0.300 wt. %, even more preferably 0.005-0.250 wt. %, most preferably 0.005-0.100 wt. %, of the total weight of the composition.
15. A composition for use according to any one of embodiments 1 to 14, wherein the fucosylated MMO is present at 0.001-0.250 wt. %, preferably 0.001-0.200 wt. %, more preferably 0.001-0.100 wt. %, even more preferably 0.001-0.020 wt. %, even more preferably 0.001-0.010 wt. %, even more preferably 0.002-0.010 wt. %, even more preferably 0.003-0.010 wt. %, even more preferably 0.003-0.0075 wt. %, most preferably 0.003-0.006 wt. %, of the total weight of the composition; preferably wherein the animal is a bovine or a horse, more preferably a calf or a foal., even more preferably a calf.
16. A composition for use according to any one of embodiments 1 to 14, wherein the fucosylated MMO is present at 0.010-1.000 wt. %, preferably 0.010-0.750 wt. %, more preferably 0.010-0.500 wt. %, even more preferably 0.010-0.400 wt. %, even more preferably 0.050-0.400 wt. %, even more preferably 0.100-0.400 wt. %, most preferably 0.100-0.300 wt. %, of the total weight of the composition; preferably wherein the animal is a pig, a sheep, a goat or a camelid (preferably llama or alpaca), more preferably wherein the animal is a piglet, a lamb, a kid or a cria, even more preferably wherein the animal is a piglet, a lamb or a kid, most preferably wherein the animal is a piglet.
17. A composition for use according to any one of embodiments 1 to 16, wherein the method comprises administering an effective amount of the fucosylated MMO.
18. A composition for use according to any one of embodiments 1 to 17, wherein the fucosylated MMO is administered to the animal at least once, preferably at least twice, between birth (0 weeks) and 5 weeks of age, preferably between birth and 4 weeks of age, more preferably between birth and 3 weeks of age.
19. A composition for use according to any one of embodiments 1 to 18, wherein the fucosylated MMO is administered to the animal at least once, preferably at least twice, more preferably daily:
   in the three weeks, preferably two weeks, more preferably one week, preceding the day of transporting the animal; and/or
   on the day of transporting the animal; and/or
   in the three weeks, preferably two weeks, more preferably one week, after the day of transporting the animal.
20. A composition for use according to any one of embodiments 1 to 19, wherein the fucosylated MMO is administered to the animal for at least 1 week, preferably at least 2 weeks, more preferably at least 3 weeks, even more preferably at least 4 weeks, most preferably at least 6 weeks.
21. A composition for use according to any one of embodiments 1 to 20, wherein the fucosylated MMO is administered to the animal for ≤32 weeks, preferably ≤28 weeks, more preferably ≤24 weeks, even more preferably ≤20 weeks, even more preferably ≤16 weeks, even more preferably ≤12 weeks, most preferably ≤6 weeks.
22. A composition for use according to any one of embodiments 1 to 21, wherein the fucosylated MMO is administered to the animal for 1-36, preferably 1-30, even more preferably 1-28, even more preferably 1-24, even more preferably 1-18, even more preferably 1-12, even more preferably 1-8, even more preferably 1-6, even more preferably 1-4, most preferably 2-4, consecutive weeks.
23. A composition for use according to any one of embodiments 1 to 22, wherein the fucosylated MMO is administered to the animal for:
   if subject is bovine: 1-36, preferably 1-32, more preferably 1-30, even more preferably 1-28, even more preferably 1-24, even more preferably 1-18, even more preferably 1-12, even more preferably 2-12, most preferably 3-12, consecutive weeks;
   if subject is a pig: 1-18, preferably 1-12, more preferably 1-8, even more preferably 1-6, even more preferably 1-4, most preferably 2-4, consecutive weeks;
   if subject is a sheep: 1-36, preferably 1-32, more preferably 1-30, even more preferably 1-28, even more preferably 1-24, even more preferably 1-18, even more preferably 1-12, even more preferably 1-8, even more preferably 1-6, even more preferably 1-4, most preferably 2-4, consecutive weeks;
   if subject is a goat: 1-36, preferably 1-32, more preferably 1-30, even more preferably 1-28, even more preferably 1-24, even more preferably 1-18, even more preferably 1-12, even more preferably 1-8, even more preferably 1-6, even more preferably 1-4, most preferably 2-4, consecutive weeks;
   if subject is a horse: 1-18, preferably 1-12, more preferably 1-8, even more preferably 1-6, even more preferably 1-4, most preferably 2-4, consecutive weeks;
   if subject is a camelid: 1-18, preferably 1-12, more preferably 1-8, even more preferably 1-6, even more preferably 1-4, most preferably 2-4, consecutive weeks;
   if subject is a rabbit: 1-12, preferably 1-8, more preferably 1-6, even more preferably 1-4, most preferably 2-4, consecutive weeks;
   if subject is a dog: 1-12, preferably 1-8, more preferably 1-6, even more preferably 1-4, most preferably 2-4, consecutive weeks;
   if subject is a cat: 1-12, preferably 1-8, more preferably 1-6, even more preferably 1-4, most preferably 2-4, consecutive weeks;
   if subject is poultry: 1-8, preferably 1-6, more preferably 1-4, most preferably 2-4, consecutive weeks.
24. A composition for use according to any one of embodiments 1 to 23, wherein the fucosylated MMO is administered at least once a week, preferably at least once every 3 days, more preferably at least once every 2 days, most preferably at least once daily.
25. A composition for use according to any one of embodiments 1 to 24, wherein the fucosylated MMO is administered at a daily dose of 0.0001-15.0 g, preferably 0.0001-10.0 g, more preferably 0.0001-5.0 g, even more preferably 0.0001-2.0 g, even more preferably 0.0001-1.0 g, even more preferably 0.0005-1.0 g, most preferably 0.0010-1.0 g.
26. A composition for use according to any one of embodiments 1 to 24, wherein the fucosylated MMO is administered at a daily dose of 0.01-15.0 g, preferably 0.01-10.0 g, more preferably 0.01-5.0 g, even more preferably 0.01-2.0 g, even more preferably 0.01-1.0 g, even more preferably 0.015-1.0 g, most preferably 0.02-1.0 g.
27. A composition for use according to any one of embodiments 1 to 24, wherein the fucosylated MMO is administered at a daily dose of 0.01-1.0 g, preferably 0.01-0.75 g, more preferably 0.01-0.65 g, even more preferably 0.01-0.60 g, even more preferably 0.01-0.50 g, even more preferably 0.015-0.50 g, even more preferably 0.015-0.40 g, even more preferably 0.015-0.30 g, even more preferably 0.015-0.20 g, most preferably 0.015-0.10 g; preferably wherein the animal is a bovine or a horse, more preferably a calf or a foal, even more preferably a calf.
28. A composition for use according to any one of embodiments 1 to 24, wherein the fucosylated MMO is administered at a daily dose of 0.01-5.0 g, preferably 0.1-2.5 g, more preferably 0.1-1.5 g, even more preferably 0.1-1.2 g, even more preferably 0.2-1.2 g, even more preferably 0.3-1.2 g, even more preferably 0.4-1.2 g, most preferably 0.5-1.2 g; preferably wherein the animal is a pig, a sheep, a goat or a camelid (preferably llama or alpaca), more preferably wherein the animal is a piglet, a lamb, a kid or a cria, even more preferably wherein the animal is a piglet, a lamb or a kid, most preferably wherein the animal is a piglet.
29. A composition for use according to any one of embodiments 1 to 28, wherein the fucosylated MMO is administered at a daily dose of 0.01-150.0 mg, preferably 0.01-125.0 mg, more preferably 0.01-100.0 mg, even more preferably 0.05-100.0 mg, even more preferably 0.1-100.0 mg, most preferably 0.1-80.0 mg, per kg bodyweight of the animal.
30. A composition for use according to any one of embodiments 1 to 28, wherein the fucosylated MMO is administered at a daily dose of 0.1-50.0 mg, preferably 0.1-30.0 mg, more preferably 0.1-25.0 mg, even more preferably 0.1-20.0 mg, even more preferably 0.1-15.0 mg, even more preferably 0.1-10.0 mg, even more preferably 0.25-10.0 mg, most preferably 0.5-10.0 mg, per kg bodyweight of the animal; preferably wherein the animal is a bovine or a horse, more preferably a calf or a foal, even more preferably a calf.
31. A composition for use according to any one of embodiments 1 to 28, wherein the fucosylated MMO is administered at a daily dose of 0.1-150.0 mg, preferably 0.1-125.0 mg, more preferably 1.0-125.0 mg, even more preferably 5.0-125.0 mg, even more preferably 5.0-100.0 mg, even more preferably 5.0-80.0 mg, most preferably 10.0-80.0 mg, per kg bodyweight of the animal; preferably wherein the animal is a pig, a sheep, a goat or a camelid (preferably llama or alpaca), more preferably wherein the animal is a piglet, a lamb, a kid or a cria, even more preferably wherein the animal is a piglet, a lamb or a kid, most preferably wherein the animal is a piglet.
32. A composition for use according to any one of embodiments 1 to 31, wherein the composition further comprises a second mammalian milk oligosaccharide (MMO), preferably wherein the second mammalian milk oligosaccharide is a sialylated MMO, more preferably wherein the second mammalian milk oligosaccharide is a sialylated MMO comprising a lactose, a lacto-N-biose (LNB) or N-acetyllactosamine (LacNAc) at its reducing end, even more preferably the sialylated MMO comprises lactose or LacNAc at is reducing end, most preferably the sialylated MMO comprises lactose at its reducing end.
33. A composition for use according to embodiment 32, wherein the second MMO is selected from the group consisting of 3′-sialyllactose (3′SL), 6′-sialyllactose (6′SL), 3,6-disialyllactose, 6,6′-disialyllactose, 8,3-disialyllactose, 3′S-2′FL, 6′S-2′FL, 3′S-3-FL, 6′S-3-FL, LST a, LST b, LST c, LST d, 3′-sialyllacto-N-biose (3′SLNB), 6′-sialyllacto-N-biose (6′SLNB), sialyl Lewis a, 3′-sialyllactosamine (3′SLacNAc), 6′-sialyllactosamine (6′SLacNAc) and sialyl Lewis x; preferably the second MMO is selected from the group consisting of 3′SL, 6′SL, LST a, LST b, LST c, LST d, 3′SLNB, 6′SLNB, sialyl Lewis a, 3′SLacNAc, 6′SLacNAc and sialyl Lewis x; more preferably the second MMO is selected from the group consisting of 3′SL, 6′SL, 3′SLNB, 6′SLNB, 3′SLacNAc and 6′SLacNAc; most preferably the second MMO is 3′-sialyllactose (3′SL) or 6′-sialyllactose (6′SL).
34. A composition for use according to embodiment 32 or 33, wherein the second MMO is 3′-sialyllactose (3′SL) or 6′-sialyllactose (6′SL).
35. A composition for use according to any one of embodiments 32 to 34, wherein the amount of the fucosylated MMO in the composition is lower than the amount of the second MMO, preferably the amount of the fucosylated MMO is 1.1 to 15.0, more preferably 1.5 to 15.0, even more preferably 2.5 to 15.0, even more preferably 5.0 to 15.0, even more preferably 5.0 to 12.5, most preferably 5.0 to 10.0, times lower than the amount of the second MMO in the composition.
36. A composition for use according to any one of embodiments 32 to 34, wherein the amount of the fucosylated MMO in the composition is lower than the amount of the second MMO, preferably the amount of the fucosylated MMO is 1.1 to 10.0, preferably 1.5-7.5, more preferably 2.5-7.5, most preferably 4.0-7.0, times lower than the amount of the second MMO in the composition.
37. A composition for use according to any one of embodiments 32 to 34, wherein the amount of the second MMO in the composition is 0.1-30.0, preferably 0.1-25.0, more preferably 0.1-20.0, even more preferably 0.1-15.0, even more preferably 0.2-15.0, even more preferably 0.3-15.0, even more preferably 0.4-15.0, most preferably 0.5-15.0, times the amount of the fucosylated MMO in the composition.
38. A composition for use according to any one of embodiments 1 to 37, wherein the composition further comprises a third mammalian milk oligosaccharide (MMO), preferably wherein the third mammalian milk oligosaccharide is a sialylated MMO, more preferably wherein the third mammalian milk oligosaccharide is a sialylated MMO comprising a lactose, a lacto-N-biose (LNB) or N-acetyllactosamine (LacNAc) at its reducing end, even more preferably the sialylated MMO comprises lactose or LacNAc at is reducing end, most preferably the sialylated MMO comprises lactose at its reducing end.
39. A composition for use according to embodiment 38, wherein the third MMO is selected from the group consisting of 3′-sialyllactose (3′SL), 6′-sialyllactose (6′SL), 3,6-disialyllactose, 6,6′-disialyllactose, 8,3-disialyllactose, 3′S-2′FL, 6′S-2′FL, 3′S-3-FL, 6′S-3-FL, LST a, LST b, LST c, LST d, 3′-sialyllacto-N-biose (3′SLNB), 6′-sialyllacto-N-biose (6′SLNB), sialyl Lewis a, 3′-sialyllactosamine (3′SLacNAc), 6′-sialyllactosamine (6′SLacNAc) and sialyl Lewis x; preferably the third MMO is selected from the group consisting of 3′SL, 6′SL, LST a, LST b, LST c, LST d, 3′SLNB, 6′SLNB, sialyl Lewis a, 3′SLacNAc, 6′SLacNAc and sialyl Lewis x; more preferably the third MMO is selected from the group consisting of 3′SL, 6′SL, 3′SLNB, 6′SLNB, 3′SLacNAc and 6′SLacNAc; most preferably the third MMO is 6′-sialyllactose (6′SL) or 3′-sialyllactose (3′SL).
40. A composition for use according to embodiment 38 or 39, wherein the third MMO is 6′-sialyllactose (6′SL) or 3′-sialyllactose (3′SL).
41. A composition for use according to any one of embodiments 38 to 40, wherein the amount of the fucosylated MMO in the composition is lower than the amount of the third MMO, preferably the amount of the fucosylated MMO is 1.1 to 15.0, more preferably 1.5 to 15.0, even more preferably 2.5 to 15.0, even more preferably 5.0 to 15.0, even more preferably 5.0 to 12.5, most preferably 5.0 to 10.0, times lower than the amount of the third MMO in the composition.
42. A composition for use according to any one of embodiments 38 to 40, wherein the amount of the fucosylated MMO in the composition is lower than the amount of the third MMO, preferably the amount of the fucosylated MMO is 1.1 to 10.0, preferably 1.5-7.5, more preferably 1.5-5.0, most preferably 2.5-5.0, times lower than the amount of the third MMO in the composition.
43. A composition for use according to any one of embodiments 38 to 40, wherein the amount of the third MMO in the composition is 0.1-30.0, preferably 0.1-25.0, more preferably 0.1-20.0, even more preferably 0.1-15.0, even more preferably 0.2-15.0, even more preferably 0.3-15.0, even more preferably 0.4-15.0, most preferably 0.5-15.0, times the amount of the fucosylated MMO in the composition.
44. A composition for use according to any one of embodiments 38 to 43, wherein the ratio between the amount of the second MMO and the amount of the third MMO in the composition is similar to this ratio as found in the mother's milk of the same species.
45. A composition for use according to any one of embodiments 38 to 44, wherein the amount of the second MMO in the composition is higher than the amount of the third MMO, preferably the amount of the second MMO is 1.1 to 40.0, more preferably 1.1 to 35.0, most preferably 1.5 to 35.0, times the amount of the third MMO in the composition.
46. A composition for use according to any one of embodiments 38 to 44, wherein the amount of the second MMO in the composition is higher than the amount of the third MMO, preferably the amount of the second MMO is 1.1 to 6.0, more preferably 1.1 to 5.0, even more preferably 1.25 to 4.0, even more preferably 1.25-3.0, even more preferably 1.5 to 3.0, most preferably 1.5-2.5, times higher than the amount of the third MMO in the composition.
47. A composition for use according to any one of embodiments 1 to 46, wherein the composition does not comprise lacto-N-tetraose (LNT).
48. A composition for use according to any one of embodiments 1 to 47, wherein the composition does not comprise lacto-N-neotetraose (LNnT).
49. A composition for use according to any one of embodiments 1 to 48, wherein the composition does not comprise N-acetyllactosamine.
50. A composition for use according to any one of embodiments 1 to 49, wherein the composition does not comprise an oligosaccharide containing N-acetyllactosamine, preferably does not comprise a saccharide containing N-acetyllactosamine.
51. A composition for use according to any one of embodiments 1 to 50, wherein the composition does not comprise a fructo-oligosaccharide (FOS).
52. A composition for use according to any one of embodiments 1 to 51, wherein the composition does not comprise a galacto-oligosaccharide (GOS).
53. A composition for use according to any one of embodiments 1 to 52, wherein the composition does not comprise an isomaltooligosaccharide.
54. A composition for use according to any one of embodiments 1 to 53, wherein the composition does not comprise a neutral non-fucosylated MMO.
55. A composition for use according to any one of embodiments 1 to 31 and 47 to 54, wherein the composition does not comprise a further mammalian milk oligosaccharide.
56. A composition for use according to any one of embodiments 1 to 55, wherein the composition further comprises a microbial strain that does not use, preferably is not capable to use, the fucosylated MMO as carbon source.
57. A composition for use according to any one of embodiments 1 to 55, wherein the composition does not comprise a microbial strain.
58. A composition for use according to any one of embodiments 1 to 57, wherein the composition is not mother's milk.
59. A composition for use according to any one of embodiments 1 to 58, wherein the composition is a synthetic composition.
60. A composition for use according to any one of embodiments 1 to 59, wherein the fucosylated MMO has been produced in vitro and/or ex vivo by a cell, preferably a single cell, wherein the cell is preferably chosen from the group consisting of a microorganism, a plant cell, an animal cell and a protozoan cell.
61. A composition for use according to any one of embodiments 1 to 60, wherein the fucosylated MMO is linked, preferably chemically linked, to a carrier for delivery of the fucosylated MMO.
62. A composition for use according to embodiment 61, wherein the linker is a ceramide-based carrier or a polypeptide-based carrier, preferably a ceramide-based carrier.
63. A composition for use according to embodiment 62, wherein the ceramide-based carrier is selected from a group consisting of d18:1/16:0, t18:0-16:0, t18:0-h16:0, t18:0-h22:0 and t18:0-h24:0.
64. A composition for use according to any one of embodiments 1 to 63, wherein the fucosylated MMO is encapsulated.
65. A composition for use according to any one of embodiments 1 to 64, wherein the composition is a pharmaceutical composition, optionally further comprising a pharmaceutically acceptable carrier, filler, preservative, solubilizer, diluent, excipient, salt, adjuvant and/or solvent.
66. A composition for use according to any one of embodiments 1 to 65, wherein the composition is a nutritional composition, optionally further comprising a feed ingredient and/or a food ingredient, wherein the feed/food ingredient is preferably chosen from the group consisting of:
   a lipid, preferably one or more selected from the group consisting of an oil, fat, ester, monoglyceride, diglyceride, triglyceride and free fatty acid;
   a vitamin, preferably one or more selected from the group consisting of vitamin A, vitamin B, vitamin C, vitamin D, vitamin E and vitamin H, or a derivate thereof;
   an amino acid compound;
   a trace element;
   a mineral., preferably one or more selected from the group consisting of calcium, phosphorus, magnesium, iron, zinc, manganese, copper, sodium, potassium, molybdenum, chromium, selenium and chloride;
   an antioxidant;
   a prebiotic agent, preferably one or more selected from the group consisting of GOS (galacto-oligosaccharide), FOS (fructo-oligosaccharide), inulin and resistant starch;
   a carbohydrate;
   an antimicrobial agent; and/or
   a protein.
67. A composition for use according to embodiment 66, wherein the nutritional composition is selected from a group consisting of a milk replacer, creep feed, prestarter diet, starter diet, pre-weaning feed, weaning feed and post-weaning feed, preferably selected form the group consisting of a milk replacer, starter diet, creep feed and post-weaning feed, more preferably the nutritional composition is a milk replacer or starter diet, most preferably a milk replacer.
68. A method for preventing and/or treating shipping fever in an animal, preferably a farm animal or a domesticated animal, more preferably a farm animal, wherein the method comprises administering a composition as defined in any one of embodiments 1 to 67.
69. A method according to embodiment 68, wherein the method is for preventing and/or treating shipping fever pneumonia and/or shipping fever diarrhea, preferably for preventing and/or treating shipping fever pneumonia.
70. A method according to embodiment 69, wherein the pneumonia is acute pneumonia.
71. A method according to any one of embodiments 68 to 70, wherein the animal is as defined in any one of embodiments 4 to 6.
72. A method according to any one of embodiments 68 to 71, wherein the method comprises administering an effective amount of the fucosylated MMO to the animal., preferably wherein the fucosylated MMO is administered as defined in any one of embodiments 18 to 31.
73. Use of a composition as defined in any one of embodiments 1 to 67 for the manufacture of a medicament for preventing and/or treating shipping fever in an animal, preferably wherein the animal is a farm animal or a domesticated animal, more preferably wherein the animal is a farm animal.
74. Use according to embodiment 73, wherein the medicament is for preventing and/or treating shipping fever pneumonia and/or shipping fever diarrhea, preferably for preventing and/or treating shipping fever pneumonia.
75. Use according to embodiment 74, wherein the pneumonia is acute pneumonia.
76. Use according to any one of embodiments 73 to 75, wherein the animal is as defined in any one of embodiments 4 to 6.
77. Use according to any one of embodiments 73 to 76, wherein the method comprises administering an effective amount of the fucosylated MMO to the animal, preferably wherein the fucosylated MMO is administered as defined in any one of embodiments 18 to 31.

This disclosure more preferably relates to the following specific embodiments:
1. A fucosylated mammalian milk oligosaccharide (MMO) for use in a method for preventing and/or treating shipping fever in an animal, preferably in a farm or domesticated animal, more preferably a farm animal.
2. A fucosylated MMO for use according to embodiment 1, wherein the method is a method for preventing and/or treating shipping fever in an animal by enhancing the T lymphocyte immunity in the animal.
3. A fucosylated MMO for use according to embodiment 1 or 2, the method is for preventing and/or treating shipping fever pneumonia and/or shipping fever diarrhea, preferably for preventing and/or treating shipping fever pneumonia.
4. A fucosylated MMO for use according to embodiment 3, wherein the pneumonia is acute pneumonia.
5. A fucosylated MMO for use according to any one of embodiments 1 to 4, wherein the animal is selected from the group consisting of bovine, pig, sheep, goat, horse, donkey, mule, hinny, camelid, dog, cat, rabbit and poultry.
6. A fucosylated MMO for use according to any one of embodiments 1 to 5, wherein the animal is a non-adult animal.
7. A fucosylated MMO for use according to any one of embodiments 1 to 6, wherein the fucosylated MMO comprises a fucose that is linked to a monosaccharide in an α-1,2-linkage, and wherein the monosaccharide is galactose.
8. A fucosylated MMO for use according to any one of embodiments 1 to 7, wherein the fucosylated MMO is 2′-fucosyllactose (2′FL).
9. A fucosylated MMO for use according to any one of embodiments 1 to 8, wherein the method does not comprise administering lacto-N-tetraose (LNT) or lacto-N-neotetraose (LNnT).
10. A fucosylated MMO for use according to any one of embodiments 1 to 9, wherein the method does not comprise administering an additional MMO.
11. A fucosylated MMO for use according to any one of embodiments 1 to 10, wherein the fucosylated MMO is administered to the animal at least once, preferably at least twice, between birth (0 weeks) and 5 weeks of age, preferably between birth and 4 weeks of age, more preferably between birth and 3 weeks of age.
12. A fucosylated MMO for use according to any one of embodiments 1 to 11, wherein the fucosylated MMO is administered to the animal at least once, preferably at least twice, more preferably daily:
   in the three weeks, preferably two weeks, more preferably one week, preceding the day of transporting the animal; and/or
   on the day of transporting the animal; and/or
   in the three weeks, preferably two weeks, more preferably one week, after the day of transporting the animal.
13. A fucosylated MMO for use according to any one of embodiments 1 to 12, wherein the fucosylated MMO is administered at a daily dose of 0.01-1.0 g, preferably 0.01-0.75 g, more preferably 0.01-0.65 g, even more preferably 0.01-0.60 g, even more preferably 0.01-0.50 g, even more preferably 0.015-0.50 g, even more preferably 0.015-0.40 g, even more preferably 0.015-0.30 g, even more preferably 0.015-0.20 g, most preferably 0.015-0.10 g; preferably wherein the animal is a bovine or a horse, more preferably a calf or a foal, even more preferably a calf.
14. A fucosylated MMO for use according to any one of embodiments 1 to 12, wherein the fucosylated MMO is administered at a daily dose of 0.01-5.0 g, preferably 0.1-2.5 g, more preferably 0.1-1.5 g, even more preferably 0.1-1.2 g, even more preferably 0.2-1.2 g, even more preferably 0.3-1.2 g, even more preferably 0.4-1.2 g, most preferably 0.5-1.2 g; preferably wherein the animal is a pig, a sheep, a goat or a camelid (preferably llama or alpaca), more preferably wherein the animal is a piglet, a lamb, a kid or a cria, even more preferably wherein the animal is a piglet, a lamb or a kid, most preferably wherein the animal is a piglet.
15. A fucosylated MMO for use according to any one of embodiments 1 to 14, wherein the fucosylated MMO is administered at a daily dose of 0.01-150.0 mg, preferably 0.01-125.0 mg, more preferably 0.01-100.0 mg, even more preferably 0.05-100.0 mg, even more preferably 0.1-100.0 mg, most preferably 0.1-80.0 mg, per kg bodyweight of the animal.
16. A fucosylated MMO for use according to any one of embodiments 1 to 14, wherein the fucosylated MMO is part of a composition.
17. A fucosylated MMO for use according to embodiment 16, wherein the composition is not mother's milk.
18. A fucosylated MMO for use according to embodiment 16 or 17, wherein the composition is a pharmaceutical composition, optionally further comprising a pharmaceutically acceptable carrier, filler, preservative, solubilizer, diluent, excipient, salt, adjuvant and/or solvent.
19. A fucosylated MMO for use according to any one of embodiments 16 to 18, wherein the composition is a nutritional composition and selected from a group consisting of a milk replacer, creep feed, prestarter diet, starter diet, pre-weaning feed, weaning feed and post-weaning feed, preferably selected form the group consisting of a milk replacer, starter diet, creep feed and post-weaning feed, more preferably the nutritional composition is a milk replacer or starter diet, most preferably a milk replacer.

### Definitions

The words used in this specification to describe the disclosure and its various embodiments are to be understood not only in the sense of their commonly defined meanings, but to include by special definition in this specification structure, material or acts beyond the scope of the commonly defined meanings. Thus, if an element can be understood in the context of this specification as including more than one meaning, then its use in a claim must be understood as being generic to all possible meanings supported by the specification and by the word itself.

The various aspects and embodiments of the disclosure disclosed herein are to be understood not only in the order and context specifically described in this specification, but to include any order and any combination thereof. Each embodiment as identified herein may be combined together unless otherwise indicated. All publications, patents and patent applications cited in this specification are herein incorporated by reference in their entirety to the same extent as if each individual publication, patent or patent application was specifically and individually indicated to be incorporated by reference in its entirety. Unless specifically stated otherwise, all words used in the singular number shall be deemed to include the plural and vice versa. Unless defined otherwise, all technical and scientific terms used herein generally have the same meaning as commonly understood by one of ordinary skill in the art to which this disclosure belongs. Generally, the nomenclature used herein and the laboratory procedures in cell culture, molecular genetics, organic chemistry and nucleic acid chemistry and hybridization described herein are those well-known and commonly employed in the art. Standard techniques are used for nucleic acid and peptide synthesis. Generally, enzymatic reactions and purification steps are performed according to the manufacturer's specifications.

In the drawings and specification, there have been disclosed embodiments of the disclosure, and although specific terms are employed, the terms are used in a descriptive sense only and not for purposes of limitation, the scope of the disclosure being set forth in the following claims. It must be understood that the illustrated embodiments have been set forth only for the purposes of example and should not be taken as limiting the disclosure. It will be apparent to those skilled in the art that alterations, other embodiments, improvements, details and uses can be made consistent with the letter and spirit of the disclosure herein and within the scope of this disclosure, which is limited only by the claims, construed in accordance with the patent law, including the doctrine of equivalents. In the claims that follow, reference characters used to designate claim steps are provided for convenience of description only, and are not intended to imply any particular order for performing the steps (unless specifically stated otherwise).

In this document and in its claims, the verbs “to comprise”, “to have” and “to contain”, and their conjugations are used in their non-limiting sense to mean that items following the word are included, but items not specifically mentioned are not excluded. The verb “to consist essentially of” means that e.g., a composition as defined herein may comprise additional component(s) than the ones specifically identified, the additional component(s) not altering the unique characteristic of the disclosure. Throughout the document and claims, unless specifically stated otherwise, the verbs “to comprise”, “to have” and “to contain”, and their conjugations, may be preferably replaced by “to consist” (and its conjugations) or “to consist essentially of” (and its conjugations) and vice versa. In addition, reference to an element by the indefinite article “a” or “an” does not exclude the possibility that more than one of the element is present, unless the context clearly requires that there be one and only one of the elements. The indefinite article “a” or “an” thus usually means “at least one”. The word “about” or “approximately” or “around” when used in association with a numerical value (e.g., about 10) preferably means that the value may be the given value (of 10) more or less 10%, preferably 5%, more preferably 1% of the value.

Throughout the description and claims, unless specifically stated otherwise, the expression “from x to y”, wherein x and y represent numerical values, refers to a range of numerical values wherein both x and y are included and wherein x represents the lowest value and y represents the highest value. Hence, both x and y are included in the range.

The terms “LNT II”, “LNT-II”, “LN3”, “lacto-N-triose II”, “lacto-N-triose II”, “lacto-N-triose”, “lacto-N-triose” and “GlcNAc-β1,3-Gal-β1,4-Glc” are used interchangeably.

The terms “LNT”, “lacto-N-tetraose”, “lacto-N-tetraose” and “Gal-β1,3-GlcNAc-β1,3-Gal-β1,4Glc” are used interchangeably.

The terms “LNnT”, “lacto-N-neotetraose”, “lacto-N-neotetraose”, “neo-LNT” and “Galβ1-4GlcNAcβ1-3Galβ1-4Glc” are used interchangeably.

The terms “2′ fucosyllactose”, “2′-fucosyllactose”, “α-1,2-fucosyllactose”, “α 1,2 fucosyllactose”, “α-1,2-fucosyllactose”, “α 1,2 fucosyllactose”, “Fuc-α1,2-Gal-β1,4-Glc”, 2FL″ and “2′FL” are used interchangeably.

The terms “3-fucosyllactose”, “α-1,3-fucosyllactose”, “α 1,3 fucosyllactose”, “α-1,3-fucosyllactose”, “α 1,3 fucosyllactose”, “Gal-β1,4-(Fuc-α1,3-)Glc”, 3FL″ and “3-FL” are used interchangeably.

The terms “difucosyllactose”, “di-fucosyllactose”, “lactodifucotetraose”, “2′,3-difucosyllactose”, “2′,3 difucosyllactose”, “α-2′,3-fucosyllactose”, “α 2′,3 fucosyllactose, “Fuc-α1,2-Gal-1,4-(Fuc-α1,3-)Glc”, “DFLac”, 2′,3 diFL “, “DFL”, “DiFL” and “diFL” are used interchangeably.

The terms “LNFP-I”, “lacto-N-fucopentaose I”, “LNFP I”, “LNF I OH type I determinant”, “LNF I”, “LNF1”, “LNF 1”, “Blood group H antigen pentaose type 1” and “Fuc-α1,2-Gal-β1,3-GlcNAc-β1,3-Gal-β1,4-Glc” are used interchangeably.

The terms “GalNAc-LNFP-I”, “blood group A antigen hexaose type I”, and “GalNAc-α1,3-(Fuc-α1,2)-Gal-β1,3-GlcNAc-β1,3-Gal-β1,4-Glc” are used interchangeably.

The terms “Gal-LNFP-I”, “blood group B antigen hexaose type I” and “Gal-α1,3-(Fuc-α1,2)-Gal-β1,3-GlcNAc-β1,3-Gal-β1,4-Glc” are used interchangeably.

The terms “LNFP-II”, “lacto-N-fucopentaose II” and “Gal-β1,3-(Fuc-α1,4)-GlcNAc-β1,3-Gal-β1,4-Glc” are used interchangeably.

The terms “LNFP-III”, “lacto-N-fucopentaose III” and “Gal-β1,4-(Fuc-α1,3)-GlcNAc-β1,3-Gal-β1,4-Glc” are used interchangeably.

The terms “LNFP-V”, “lacto-N-fucopentaose V” and “Gal-β1,3-GlcNAc-β1,3-Gal-β1,4-(Fuc-α1,3)-Glc” are used interchangeably.

The terms “LNDFH I”, “Lacto-N-difucohexaose I”, “LNDFH-I”, “LDFH I”, “Leb-lactose”, “Lewis-b hexasaccharide” and “Fuc-α1,2-Gal-β1,3-[Fuc-α1,4]-GlcNAc-β1,3-Gal-β1,4-Glc” are used interchangeably.

The terms “LNDFH II”, “Lacto-N-difucohexaose II”, “Lewis a-Lewis x”, “LDFH II” and “Fuc-α1,4-(Gal-β1,3)-GlcNAc-β1,3-Gal-β1,4-(Fuc-α1,3)-Glc” are used interchangeably.

The terms “lewis b-lewis x” and “Fucα1,4-[Fuc-α1,2-Galβ1,3]-GlcNAc-β1,3-Gal-β1,4-[Fuc-α1,3]-Glc are used interchangeably.

The terms “MFLNH III”, “monofucosyllacto-N-hexaose-III” and “Gal-β1,4-[Fuc-α1,3]-GlcNAc-β1,6-[Gal-β1,3-GlcNAc-β1,3]-Gal-β1,4-Glc” are used interchangeably.

The terms “DFLNH (a)”, “difucosyllacto-N-hexaose (a)” and “Gal-β1,4-[Fuc-α1,3]-GlcNAc-β1,6-[Fuc-α1,2-Gal-β1,3-GlcNAc-β1,3]-Gal-β1,4-Glc” are used interchangeably.

The terms “DFLNH”, “difucosyllacto-N-hexaose” and “Gal-β1,4-[Fuc-α1,3]-GlcNAc-β1,6-[Fuc-α1,4-[Gal-β1,3]-GlcNAc-β1,3]-Gal-β1,4-Glc” are used interchangeably.

The terms “TFLNH”, “trifucosyllacto-N-hexaose” and “Gal-β1,4-[Fuc-α1,3]-GlcNAc-β1,6-[Fuc-α1,4-[Fuc-α1,2-Gal-β1,3]-GlcNAc-β1,3]-Gal-β1,4-Glc” are used interchangeably.

The terms “LNnFP I”, “Lacto-N-neofucopentaose I” and “Fuc-α1,2-Gal-1,4-GlcNAc-β1,3-Gal-β1,4-Glc” are used interchangeably.

The terms “LNFP-VI”, “LNnFP V”, “lacto-N-neofucopentaose V” and “Gal-β1,4-GlcNAc-β1,3-Gal-β1,4-(Fuc-α1,3)-Glc” are used interchangeably.

The terms “LNnDFH”, “Lacto-N-neoDiFucohexaose”, “Lewis x hexaose” “Gal-β1,4-(Fuc-α1,3)-GlcNAc-β1,3-Gal-β1,4-(Fuc-α1,3)-Glc” are used interchangeably.

The terms “2′-fucosyllacto-N-biose”, “2′FLNB” and “Fuc-α1,2-Gal-β1,3-GlcNAc” are used interchangeably.

The terms “4-fucosyllacto-N-biose”, “4FLNB” and “Fuc-α1,4-[Gal-β1,3-]GlcNAc” are used interchangeably.

The terms “difucosyllacto-N-biose”, “diFLNB” and “Fuc-α1,4-[Fuc-α1,2-Gal-β1,3-] GlcNAc” are used interchangeably.

The terms “2′-fucosyl-N-acetyllactosamine”, “2′FlacNAc” and “Fuc-α1,2-Gal-β1,4-GlcNAc” are used interchangeably.

The terms “3-fucosyl-N-acetyllactosamine”, “3FlacNAc” and “Gal-1,4-(Fuc-α1,3-)GlcNAc” are used interchangeably.

The terms “difucosyl-N-acetyllactosamine”, “diFlacNAc” and “Fuc-α1,2-Gal-β1,4-[Fuc-α1,3-]GlcNAc” are used interchangeably.

The terms “3′ sialyllactose”, “3′-sialyllactose”, “α-2,3-sialyllactose”, “α 2,3 sialyllactose”, “α-2,3-sialyllactose”, “α 2,3 sialyllactose”, “3SL”, “Sia-α2,3-Gal-β1,4-Glc” and “3′SL” are used interchangeably.

The terms “6′ sialyllactose”, “6′-sialyllactose”, “α-2,6-sialyllactose”, “α 2,6 sialyllactose”, “α-2,6-sialyllactose”, “α 2,6 sialyllactose”, “6SL”, “Sia-α2,6-Gal-β1,4-Glc” and “6′SL” are used interchangeably.

The terms “3,6-disialyllactose” and “Neu5Ac-α2,3-Neu5Ac-α2,6-Gal-β1,4-Glc” are used interchangeably.

The terms “6,6′-disialyllactose” and “Neu5Ac-α2,6-Neu5Ac-α2,6-Gal-β1,4-Glc” are used interchangeably.

The terms “8,3-disialyllactose” and “Neu5Ac-α2,8-Neu5Ac-α2,3-Gal-β1,4-Glc” are used interchangeably.

The terms “3′S-2′FL” and “Neu5Ac-α2,3-[Fuc-α1,2-]Gal-β1,4-Glc” are used interchangeably.

The terms “6′S-2′FL” and “Neu5Ac-α2,6-[Fuc-α1,2-]Gal-β1,4-Glc” are used interchangeably.

The terms “3′S-3-FL” and “Neu5Ac-α2,3-Gal-β1,4-[Fuc-α1,3]Glc” are used interchangeably.

The terms “6′S-3-FL” and “Neu5Ac-α2,6-Gal-β1,4-[Fuc-α1,3]Glc” are used interchangeably.

The terms “LSTa”, “LS-Tetrasaccharide a”, “Sialyl-lacto-N-tetraose a”, “sialyllacto-N-tetraose a” and “Neu5Ac-α2,3-Gal-b1,3-GlcNAc-b1,3-Gal-b1,4-Glc” are used interchangeably

The terms “LSTb”, “LS-Tetrasaccharide b”, “Sialyl-lacto-N-tetraose b”, “sialyllacto-N-tetraose b” and “Gal-b1,3-(Neu5Ac-α2,6)-GlcNAc-b1,3-Gal-b1,4-Glc” are used interchangeably.

The terms “LSTc”, “LS-Tetrasaccharide c”, “Sialyl-lacto-N-tetraose c”, “sialyllacto-N-tetraose c”, “sialyllacto-N-neotetraose c” and “Neu5Ac-α2,6-Gal-b1,4-GlcNAc-b1,3-Gal-b1,4-Glc” are used interchangeably.

The terms “LSTd”, “LS-Tetrasaccharide d”, “Sialyl-lacto-N-tetraose d”, “sialyllacto-N-tetraose d”, “sialyllacto-N-neotetraose d” and “Neu5Ac-α2,3-Gal-b1,4-GlcNAc-b1,3-Gal-b1,4-Glc” are used interchangeably.

The terms “3′-sialyllacto-N-biose”, “3′SLNB” and “Neu5Ac-α2,3-Gal-b1,3-GlcNAc” are used interchangeably.

The terms “6′-sialyllacto-N-biose”, “6′SLNB” and “Neu5Ac-α2,6-Gal-b1,3-GlcNAc” are used interchangeably.

The terms “monofucosylmonosialyllacto-N-octaose”, “sialyl Lewis a”, “sialyl Lea”, “5-acetylneuraminyl-(2-3)-galactosyl-(1-3)-(fucopyranosyl-(1-4))-N-acetylglucosamine” and “Neu5Ac-α2,3-Gal-β1,3-[Fuc-α1,4]-GlcNAc” are used interchangeably.

The terms “3′-sialyllactosamine”, “3′SLacNAc” and “Neu5Ac-α2,3-Gal-b1,4-GlcNAc” are used interchangeably.

The terms “6′-sialyllactosamine”, “6′SLacNAc” and “Neu5Ac-α2,6-Gal-b1,4-GlcNAc” are used interchangeably.

The terms “sialyl Lewis x”, “sialyl Lex”, “5-acetylneuraminyl-(2-3)-galactosyl-(1-4)-(fucopyranosyl-(1-3))-N-acetylglucosamine” and “Neu5Ac-α2,3-Gal-β1,4-[Fuc-α1,3-]GlcNAc” are used interchangeably.

The term “cultivation” refers to the culture medium wherein the cell is cultivated or fermented, the cell itself, and the saccharide(s) that is/are produced by the cell in whole broth, i.e., inside (intracellularly) as well as outside (extracellularly) of the cell.

The term “clarifying” refers to the act of treating an aqueous solution to remove suspended particulates and contaminants from the production process, like e.g., cells, cell components, insoluble metabolites and debris that could interfere with the eventual purification of the oligosaccharide(s) of interest. Such treatment can be carried out in a conventional manner by centrifugation, flocculation, flocculation with optional ultrasonic treatment, gravity filtration, microfiltration, foam separation or vacuum filtration (e.g., through a ceramic filter, which can include a Celite™ filter aid).

### EXAMPLES

This disclosure will be described in more detail in the Examples. The following examples will serve as further illustration and clarification of the disclosure and are not intended to be limiting in any way.

### Example 1: 2′FL

2′-fucosyllactose (2′FL) was recombinantly produced in E. coli and purified as described in examples 10, 13 and 14 of WO 2022/034079, and subsequently spray dried as described in Example 21 of WO 2022/034079 to obtain 2″FL powder (purity 89.6%).

### Example 2: Trials in Calves

### Animals and Diets

Holstein Friesian calves (2-3 week-old, 95% male) were transported from different farms to the experimental facility of Denkavit (Voorthuizen, The Netherlands) where the animals were reared. The experiment was performed in two trial rounds.

### Round 1: Veal Calf

The calves were randomly and gender-equally divided in two groups based on experimental calf milk replacer: group A (placebo-standard calf milk replacer (CMR) powder of the farm) and group B (standard CMR powder supplemented with 0.005% drywt. 2′-fucosyllactose (2′FL, see Example 1). The calf milk replacer was administered as a liquid to the calves (water was added to the CMR powder to obtain 150 gram CMR powder per liter liquid CMR). Each group involved 290 calves, assigned into 12 rows. Each row contained 4 pens, where six calves were living in one pen.

2′-fucosyllactose was supplied to calves via CMR (major components are presented in Table 1) during the whole trial, lasting 6 months. The Start CMR was given from arrival till week 6, which then switched to the Gold Alfa CMR up to day 136. The remaining time calves were fed with the Omega CMR. Solid feed (concentrates) was introduced gradually from arrival, starting with the Avance Alfa up till day 126, then replaced by the Avance Omega till the end of the experiment. Feed was provided ad libitum to calves twice a day (morning and evening) and were recorded daily based on rows (concentrates, straw, corn silage) or groups (CMR) consumption.

TABLE 1
Major components of calf's standard diets in the round 1 trial

| | Protein | Fat | Carbohydrate | Ash |
|---|---|---|---|---|
| Diet | (drywt. %) | (drywt. %) | (drywt. %) | (drywt. %) |
| Start calf milk | 24.1 | 20.8 | 44 | 8.3 |
| replacer | | | | |
| Gold Alpha CMR | 18.0 | 18.7 | 54 | 7.4 |
| Gold Omega CMR | 13.7 | 19.2 | 58 | 6.8 |
| Avance Alfa | 16.7 | 4.4 | 57.7 | |
| Avance Omega | 14.6 | 4.4 | 59.5 | |

### Round 2: Rearing Calf

The calves were randomly and gender-equally divided in two groups based on experimental calf milk replacer (CMR): group A (placebo-standard CMR powder of the farm), group B (standard CMR powder supplemented with 0.005% drywt. 2′FL). The calf milk replacer was administered as a liquid to the calves (water was added to the CMR powder to obtain 150 gram CMR powder per liter liquid CMR). Each group involved 100 calves, assigned into four rows (25 calves per row) of two compartment blocks. Each row contained five pens, where four to six calves were living in one pen.

Starting from the day of arrival, calves received standard calf milk replacer (CMR) during a period of 9 weeks. Group A only received the CMR, while the CMR supplied to groups B was supplemented with 0.005% drywt. 2′FL. Calf starter (concentrates) and straw were introduced gradually from day 6 till the end of the experiment (12 weeks) while calves had access to corn silage at weeks 11 and 12. Feed were provided ad libitum to calves twice a day (morning and evening) and were recorded daily based on rows (concentrates, straw, corn silage) or groups (CMR) consumption. Major components of standard CMR and starter diets are presented in Table 2.

TABLE 2
Major components of calf's standard diets in the round 2 trial

| | Protein | Fat | Carbohydrate | Crude fiber | Ash |
|---|---|---|---|---|---|
| Diet | (Drywt. %) | (Drywt. %) | (Drywt. %) | (Drywt. %) | (Drywt. %) |
| Calf milk | 22 | 18 | 49 | | |
| replacer | Whey powder, vegetable oils (palm, coco, rapeseed), | | | | |
| | hydrolyzed wheat gluten, dextrose, premix | | | | |
| Calf starter | 17.5 | 2.2 | | 5.8 | 6.9 |

Corn, soybean meal., beet pulp, barley, wheat, whey powder,
sugar, calcium carbonate, lignocellulose, premix

### Health, Safety and Performance Observation

Calves were followed up by the farm staffs since arrival till the end of the trial. Fecal consistency in scale 1-4 (Larson et al., 1977, J. Dairy Science, 60 (6): p. 989-991) was recorded daily for the first ten days since arrival. Number of mortalities and signs of adverse effects were registered daily. The animals were treated prophylactically or metaphylactically (herd treatment) with 1 g oxytetracycline 100% (at day 4), 0.5 g Doxylin 50% WSP and 0.8 g bromhexine 2% (at day 13), and 3.1 ml Tilmicosol 250 mg/ml (at day 24) to prevent and/or treat acute lung inflammation. Individual treatments were followed by remark of the farm staffs and diagnostic of farm veterinary practitioners. Disease incidences and medicinal usage based on individual treatments were recorded to compare between treatment groups. Live weight of the animals was monitored at arrival (d1), two weeks (W2), six weeks (W6), ten weeks (W10) and 12 weeks (W12) and 17 weeks (W17) since arrival. Feed conversion ratio (FCR) of each period was computed based on the input of daily weight gain and daily feed intake.

### Pneumonia Assessment

Acute pneumonia is notified by the farm staff and evaluated by the farm veterinary practitioners based on some or all of the following symptoms (Wolfger et al., 2015, Veterinary Clinics of North America Food Animal 31, 351-365): depression; anorexia; nasal discharge; ocular discharge; cough; increased respiratory rate; increased respiratory noise on chest auscultation; and pyrexia (fever).

Serodiagnosis of Respiratory Pathogens of Calves with Acute Pneumonia

Blood was collected from jugular vein of 11 sick calves at W2 and W10. One ml of blood was aliquoted and kept at room temperature for 1 h for clotting. The clotted blood was transported to laboratory under cool condition (6-10° C.). Upon arrival at laboratory, blood was centrifuged at 2500 rpm for 5 min then supernatant collected before was heat treated at 56° C. for 30 min and stored at −80° C. Serum samples were applied in multiscreen Ab-ELISA test kit to detect antibodies of bovine respiratory syncytial virus (BRSV), parainfluenza 3 virus (BPI3), Mycoplasma bovis, Mannheimia haemolytica, adenovirus type 3 (ADENO 3), and in monoscreen Ab-ELISA test kits to detect antibodies of BoHV1 (Bovine Rhinotracheitis) or BVDV (Bovine Virus Diarrhea) (BioX Dianogstics, Rochefort, Belgium) according to the guidance of the manufacturer.

### Jejunal Sampling, RNA Extraction and RNAseq (Transcriptomics)

One healthy and middle weight calf of one pen per group was picked for euthanization in the slaughterhouse at day 71 (W11) (8 calves in total, 4 per group). A 20 cm segment of jejunum without Peyer's patch was excised and washed three times with physiological serum using a 30 ml syringe. A small segment (2-3 mm) was further excised and stored in 2 ml RNA protect tissue reagent (Qiagen) containing RNA preservation buffer for transportation at room temperature before long-term storage at −80° C.

Jejunal specimens stored in RNA protect tissue reagent were excised further to transfer 50 mg tissue into 2 ml tubes. The tissues were disrupted and homogenized using one stainless steel bead 5 mm in 900 μl QIAzol lysis reagent (QIAgen) in combination with a FASTPREP® 24 device. The homogenates were further manipulated according to the guidance of the manufacturer of the RNeasy plus universal tissue mini kit (QIAgen). The total purified RNA were eluted in 40 μl and quickly checked for the concentration and quality using Nanodrop (Implen NANOPHOTOMETER®) before storage at −80° C.

Total RNA was submitted to Azenta (Genewiz, Germany) to perform RNA sequencing. The total RNA was checked again for quality and concentration using Qubit and fragment analyzer before the mRNA were selected for polyA library preparation and sequenced using the ILLUMINA® Novaseq™ 2×150 bp technology platform guaranteed 20 million paired-end reads per samples (40 million reads).

RNA sequencing reads were quality-checked with fastqc (bioinformatics.babraham.ac.uk/projects/fastqc/) and trimmed with Trimmomatic (Bolger et al., 2014, Bioinformatics 30 (15): p. 2114-2120). Reads were aligned to the cow reference (Bos_taurus_UMD_3.1.1) using the genome alignment method in STAR (Dobin et al., 2013, Bioinformatics 29 (1): p. 15-21); Gene expression was quantified by featureCounts (Liao et al., 2014, Bioinformatics 30 (7): p. 923-930) and differential expression analysis was performed using DEseq2 (Love et al., 2014, Genome Biol. 15 (2): 550). Enrichment analysis of differential expressed was done in clusterProfiler (Wu et al., 2021, Innovation 2 (3): 100141) based on Gene Ontology.

### Statistical Analysis

Odds ratios and risk ratios of the pneumonia and diarrhea incidence were evaluated with the epitools R package using the Wald test (cran.r-project.org/web/packages/epitools/epitools.pdf). Pneumonia incidence of two rounds of trials was compared between the different treatment groups using Mantel-Haenszel chi-squared test with correction for trial using the epitools R package. Incidence plots were created with the survival R package (cran.r-project.org/web/packages/survival/index.html). Differences in mortality were evaluated using the Kaplan-Meier test in the survival package.

### Results

In term of safety, there were no adverse effects from the mammalian milk oligosaccharide as registered by the farm staffs and hence considered safe for the current target animals. In round 1, there were 21 calves dead during 6 months of the trial., of which 12 (4.14%) from group A, and 9 (3.1%) from group B. This ratio is seen normally in most of farms (<5%). There is no significant difference in mortality between the experimental groups. In round 2, during the 12 weeks trial, 7/400 calves died with no significant difference in mortality between the different groups.

Due to transportation from different farms and mingles upon arrival at the rearing facility, calves might be stressful and susceptible to diseases, specifically shipping fever. Two main shipping fever associated health issues were followed in the trials: acute pneumonia and diarrhea. Calves mainly got sick in the airway, specifically acute pneumonia (table 5).

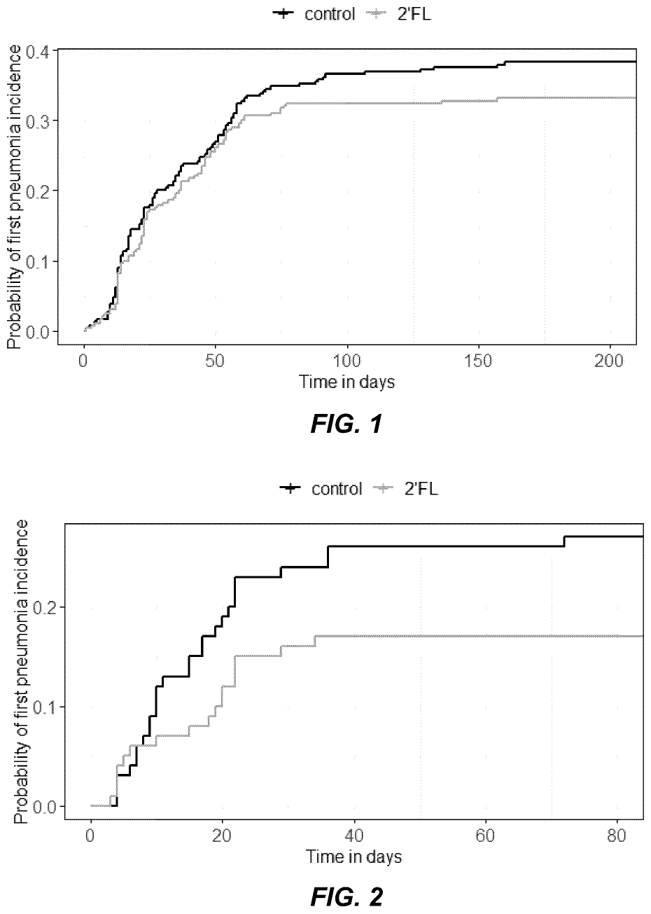

In round 1, number of diagnostic cases with acute pneumonia of the 2′FL-treated group (B; 96 cases out of 290 calves) was lower as compared to the placebo (A; 111 cases out of 290 calves). The relative risk of getting acute pneumonia in group B versus the placebo is 0.86. FIG. 1 depicts probability of first pneumonia incidence in both groups of the veal calves trial. Diagnostic assays performed on the sera of calves with symptoms of acute pneumonia pinpointed multiple causative respiratory pathogens (Table 3), such as Mycoplasma bovis and adenovirus type 3 (ADENO 3) based on frank seroconversion from at least + to +++ or ++ to ++++, less potentially by bovine respiratory syncytial virus (BRSV), parainfluenza 3 virus (BPI3), or Mannheimia haemolytica, but not BoHV1 (Bovine Rhinotracheitis) nor BVDV (Bovine Virus Diarrhea).

TABLE 3
Serodiagnosis of respiratory pathogens for blood sera of calves suffering from shipping
fever at week 2 (W2) and week 10 (W10) upon arrival in the trial facility.

| Calf | Timepoint | BRSV | BPI3 | M BOVIS | MANNHEIIA | ADENO 3 | BoHV1 | BVDV |
|---|---|---|---|---|---|---|---|---|
| 1 | W2 | ++ | ++ | 0 | 0 | + | 0 | 0 |
| | W10 | ++ | + | + | 0 | ++++ | 0 | 0 |
| 2 | W2 | + | + | 0 | + | ++ | 0 | 0 |
| | W10 | 0 | 0 | +++ | 0 | 0 | 0 | 0 |
| 3 | W2 | ++ | ++ | 0 | + | ++ | 0 | 0 |
| | W10 | ++ | + | +++ | 0 | + | 0 | 0 |
| 4 | W2 | + | + | 0 | + | +++ | 0 | 0 |
| | W10 | 0 | + | +++ | + | 0 | 0 | 0 |
| 5 | W2 | + | ++ | 0 | 0 | 0 | 0 | 0 |
| | W10 | ++ | ++ | + | 0 | ++ | 0 | 0 |
| 6 | W2 | +++ | +++ | 0 | + | +++ | 0 | 0 |
| | W10 | ++ | + | ++ | 0 | ++++ | 0 | 0 |
| 7 | W2 | +++ | +++ | + | + | +++ | 0 | ++ |
| | W10 | 0 | + | + | 0 | +++ | 0 | 0 |
| 8 | W2 | ++ | ++ | 0 | + | + | 0 | ++ |
| | W10 | + | + | + | + | +++++ | 0 | 0 |
| 9 | W2 | +++ | +++ | 0 | 0 | ++ | 0 | 0 |
| | W10 | ++ | 0 | ++ | 0 | +++ | 0 | 0 |
| 10 | W2 | ++ | +++ | 0 | + | ++++ | 0 | 0 |
| | W10 | ++ | 0 | ++ | 0 | +++++ | 0 | 0 |
| 11 | W2 | +++ | + | 0 | + | +++ | 0 | 0 |
| | W10 | ++ | + | ++++ | 0 | ++++ | 0 | 0 |

In round 2, acute pneumonia affected most of the diseased calves with 27 cases in the placebo (group A; 27%) and 17 cases in the 2′FL-treated group (B; 17%). The relative risk of getting acute pneumonia in group B versus the placebo is 0.63. FIG. 2 shows probability of first pneumonia incidence in both groups of the rearing calves trial.

Following differential expression analysis, enrichment analysis of differential expressed genes (DEGs) and ranked genes were performed. As compared to the placebo, 2′FL-treated group enhanced immune responses in several ways (T cell activation, cell differentiation, cell-cell adhesion, adaptive immune response) (Table 4). Such immune modulation supports the animals combatting against pathogens better, explaining less acute pneumonia in 2′FL-treated group, especially in the context of shipping fever wherein animals typically have a lower immunity due to the transport-related stress and hence are more prone to pathogenic infections (which is even more pronounced as the animals are in close proximity with each other).

TABLE 4
Top 20 gene ontology biological processes up-regulated in the jejunum
of 2′FL-supplemented calves (group B) versus the placebo (group A)

| Description | p. adjust |
|---|---|
| ADAPTIVE_IMMUNE_RESPONSE | 4.15E−06 |
| T_CELL_ACTIVATION | 9.06E−06 |
| MONONUCLEAR_CELL_DIFFERENTIATION | 0.000274 |
| T_CELL_RECEPTOR_COMPLEX | 0.000274 |
| T_CELL_DIFFERENTIATION | 0.000662 |
| EXTERNAL_SIDE_OF_PLASMA_MEMBRANE | 0.000673 |
| NEGATIVE_REGULATION_OF_IMMUNE_SYSTEM_PROCESS | 0.001169 |
| T_CELL_PROLIFERATION | 0.002368 |
| NEGATIVE_REGULATION_OF_ADAPTIVE_IMMUNE_RESPONSE | 0.002368 |
| NEGATIVE_REGULATION_OF_LEUKOCYTE_MEDIATED_IMMUNITY | 0.002368 |
| REGULATION_OF_ADAPTIVE_IMMUNE_RESPONSE | 0.003206 |
| POSITIVE_T_CELL_SELECTION | 0.003206 |
| LEUKOCYTE_DIFFERENTIATION | 0.003462 |
| HETEROTYPIC_CELL_CELL_ADHESION | 0.003478 |
| LYMPHOCYTE_MEDIATED_IMMUNITY | 0.003478 |
| REGULATION_OF_LEUKOCYTE_MEDIATED_IMMUNITY | 0.003478 |
| REGULATION_OF_T_CELL_ACTIVATION | 0.003668 |
| NEGATIVE_REGULATION_OF_LYMPHOCYTE_MEDIATED_IMMUNITY | 0.00552 |
| POSITIVE_THYMIC_T_CELL_SELECTION | 0.00573 |
| ADAPTIVE_IMMUNE_RESPONSE_BASED_ON_SOMATIC_RECOMBINA- | 0.00573 |
| TION_OF_IMMUNE_RECEPTORS_BUILT_FROM_IMMUNOGLOBULIN_SUPER- | |
| FAMILY_DOMAINS | |

When trials in two rounds were integrated for evaluating the efficacy of 2′FL supplementation on pneumonia incidence, the common odds ratio was computed with the value of 1.35 (P-value=0.064). It means that calves that received the standard CMR get acute pneumonia 1.35 times more often than calves that received 2′FL supplemented CMR. This strongly indicates that 2′FL-treated calves were at lower risk getting acute pneumonia as compared to the control.

TABLE 5
Acute pneumonia cases in the trials

| | Placebo | 2′FL | Relative | Common | |
|---|---|---|---|---|---|
| Trial | (A) | (B) | risk | odds ratio | P-value |
| Round 1 (veal calf) | 111 | 96 | 0.86 | 1.35 | 0.064 |
| Round 2 (rearing calf) | 27 | 17 | 0.63 | | |

Diarrhea mainly happened during the first month upon arrival (table 6). In round 1, again number of diarrhea cases in the 2′FL-treated group (B; 36 out of 290 calves) was lower than in the placebo (group A; 51 out of 290 calves). The relative risk of getting diarrhea in group B versus the placebo is 0.71. Unlike round 1, there were rarely diarrhea cases in the round 2. Only one case was recorded in the placebo group for the whole trial. Therefore, relative risk and odds ratio associated with diarrhea were computed without taking round 2 into account. The odds ratio associated with diarrhea based on the round 1 trial was computed with the value of 1.51 (P-value=0.081). It means that calves that received the standard CMR get diarrhea 1.51 times more often than calves received 2′FL supplemented CMR. This strongly indicates that 2′FL-treated calves were at lower risk getting diarrhea as compared to the control.

TABLE 6
Diarrhea cases in the trials

| | Placebo | 2′FL | Relative | | |
|---|---|---|---|---|---|
| Trial | (A) | (B) | risk | Odds ratio | P-value |
| Round 1 (veal calf) | 51 | 36 | 0.71 | 1.51 | 0.081 |
| Round 2 (rearing calf) | 1 | 0 | Not | Not | |
| | | | available | available | |

1.-19. (canceled)

20. A method for preventing or treating shipping fever in an animal, the method comprising:
administering a fucosylated mammalian milk oligosaccharide (MMO) to the animal.

21. The method according to claim 20, wherein the fucosylated MMO is administered in an amount sufficient to enhance T lymphocyte immunity in the animal.

22. The method according to claim 20, wherein the fucosylated MMO comprises a fucose that is linked to a monosaccharide in an α-1,2-linkage, and wherein the monosaccharide is galactose.

23. The method according to claim 20, wherein the fucosylated MMO is 2′-fucosyllactose (2′FL).

24. The method according to claim 20, wherein the method does not comprise administering 6′sialyllactose.

25. The method according to claim 20, wherein the method does not comprise administering a sialylated MMO.

26. The method according to claim 20, wherein the method does not comprise administering an additional MMO.

27. The method according to claim 20, wherein the animal is a non-adult animal.

28. The method according to claim 20, wherein the animal is a farm animal.

29. The method according to claim 20, wherein the animal is selected from the group consisting of bovine, pig, sheep, goat, horse, donkey, mule, hinny, camelid, dog, cat, rabbit, and poultry.

30. The method according to claim 20, wherein the fucosylated MMO is administered to the animal at least once:
in the three weeks preceding the day of the animal being transported;
on the day of the animal being transported; and/or
in the three weeks after the day of transporting the animal.

31. The method according to claim 20, wherein the fucosylated MMO is administered at a daily dose of 0.01-1.0 g.

32. The method according to claim 31, wherein the fucosylated MMO is administered at a daily dose of 0.015-0.10 g.

33. The method according to claim 31, wherein the animal is a bovine or a horse.

34. The method according to claim 20, wherein the fucosylated MMO is administered at a daily dose of 0.01-5.0 g.

35. The method according to claim 34, wherein the fucosylated MMO is administered at a daily dose of 0.5-1.2 g.

36. The method according to claim 34, wherein the animal is a pig, a sheep, a goat, or a camelid.

37. The method according to claim 20, wherein the fucosylated MMO is part of a composition.

38. The method according to claim 37, wherein the composition is formulated as a pharmaceutical composition, which optionally further comprises a pharmaceutically acceptable carrier, filler, preservative, solubilizer, diluent, excipient, salt, adjuvant, and/or solvent.

39. The method according to claim 37, wherein the composition is a nutritional composition, optionally selected from the group consisting of a milk replacer, creep feed, pre-starter diet, starter diet, pre-weaning feed, weaning feed, and post-weaning feed.
